(19) United States
(12) Patent Application Publication
(10) Pub. No.: US 2026/0273055 A1
(43) Pub. Date: Sep. 17, 2026

(54) UTILIZING T CELLS DERIVED FROM TUMOR DRAINING LYMPH NODES FOR CHIMERIC ANTIGEN RECEPTOR (CAR) T CELL THERAPY FOR THE TREATMENT OF CANCER

(71) Applicant: Cornell University, lthaca, NY (US)

(72) Inventor: Jonathan Villena-Vargas, New York, NY (US)

(21) Appl. No.: 19/168,008

(22) PCT Filed: Mar. 22, 2024
(86) PCT No.: PCT/US2024/021164

§ 371 Date: Sep. 23, 2025

Related U.S. Application Data
(60) Provisional application No. 63/454,377, filed on Mar. 24, 2023.

(51) Int. Cl. A61K 40/31; A61K 39/395; A61K 40/41; A61P 35/00; C07K 14/705; C07K 16/28

(52) U.S. Cl. CPC A61K 40/31; A61K 39/3955; A61K 40/41; A61P 35/00; C07K 14/7051; C07K 14/70517; C07K 14/70521; C07K 14/70578; C07K 16/2821; C07K 16/2845

(57) ABSTRACT

Disclosed are compositions and methods for targeted treatment of cancer. The present disclosure provides CAR therapy comprising cells from a patient's tumor draining lymph node expressing a chimeric antigen receptor.

### CROSS-REFERENCE TO RELATED APPLICATIONS

This application claims the benefit of U.S. Provisional Application No. 63/454,377, filed on Mar. 24, 2023, the entire contents of which are incorporated herein in their entirety by this reference.

### BACKGROUND

Adoptive T-cell therapy is a powerful technology for the treatment of cancer. Initial studies utilized tumor-infiltrating lymphocytes (TILs) for the treatment of solid cancers. These are made up of T cells derived from solid tumors of patients, which are then harvested, expanded, and reintroduced to the patient. This therapy showed powerful antitumor responses in a select few patients however, the majority of the T-cell responses were short-lived, variable, and unpredictable. The reason for the short-lived response was thought to be due to the exhausted state of the TILs not allowing for adequate memory T cell phenotypes with the majority of cells expressing senescence or exhaustion.

CAR T-cell therapy is fundamentally different and utilizes a chimeric antigen receptor made up of a monoclonal antibody fused to a T-cell activating domain. These are transduced to the T cells derived from the peripheral blood of patients, expanded, and given to the patients. The shortcomings of CAR T-cell therapy are the lack of endogenous tumor antigen recognition, and only displaying T-cell activation of pre-determine cell surface receptor target with the ScFv of the monoclonal antibody. Therefore, a heterogenous solid tumor may only have a partial response. An additional barrier is the trafficking of CAR T cells towards a solid tumor, including infiltration that may be due to T-cells derived from peripheral blood.

Thus, there is a need in the art for improved adoptive immunotherapy for the treatment of cancer.

### SUMMARY

The present application is based, at least in part, on the discovery that lymphocytes derived from tumor-draining lymph nodes (tdLN or TDLN) are especially effective in CAR therapy. For example, CAR T therapy using the T cells derived from TDLN are capable of recognizing endogenous tumor antigens and thus is surprisingly effective in targeting heterogenous tumors expressing various tumor antigens. When engineered with an antigen-binding domain, e.g., ICAM-1 binding domain, the CAR T cells have the added capability of robustly targeting tumors. In addition, CAR T cells prepared using the T cells derived from TDLN are surprisingly effective in targeting and infiltrating solid tumors, unlike those prepared using the T cells derived from peripheral blood.

In certain aspects, the present disclosure utilizes TDLN for CAR T cell therapy. Presented herein are data showing multiple memory phenotypes found in the tumor-draining lymph nodes in the murine model of lung cancer and benign tumor-draining lymph nodes of early-stage lung cancer patients. These nodal T cells have “stem-cell-like” properties and importantly, are specific to endogenous tumor antigens. These T cells are almost exclusively found in the TDLN and are not in the peripheral blood or the tumor of patients. As demonstrated herein, T cells from TDLN can be harvested and expanded. Utilization of these nodal T cells allows for a robust memory phenotype of CAR T cells that are capable of recognizing multiple endogenous tumor antigens in addition to a pre-determined CAR T cell target antigen.

The infusion of disease-targeting T cells as a therapeutic agent has demonstrated remarkable potential to treat advanced-stage cancers. Within adoptive cell therapy (ACT), two major strategies: (a) chimeric antigen receptor (CAR) T cells and (b) expansion of tumor infiltration lymphocytes (TILs) are currently in the clinic. However, barriers to ACT in solid tumors: 1) tumor antigen heterogeneity and 2) an immune inhibitory micro-environment leading to T cell exhaustion have impeded efficacy with either approach. Overcoming these barriers is a novel strategy of ACT of the present disclosure: Utilizing T cells derived from the patient's tumor-draining lymph nodes as a novel form of T cell therapy (FIG. 18). The results presented herein have identified tumor antigen-specific stem-cell like (SCM) memory CD8 T cell population found almost exclusively in the benign lymph nodes of non-small cell lung cancer (NSCLC) patients. In contrast to TILs where harvested T cells are thought to be terminally differentiated and show signs of T cell exhaustion and senescence, T-cells selected from lymph node acquisition (TSLA) are surprisingly 1) in a more naïve, “stem cell-like” state capable of persistence and T cell memory differentiation and 2) composed of polyclonal T cells capable of targeting multiple tumor antigens, therefore overcoming the solid tumor challenge of high antigen heterogeneity. Critically, the present disclosure demonstrates that almost all patients with early or advanced non-small cell lung cancer (NSCLC) have easily accessible mediastinal and hilar adenopathy that can be reproducibly harvested, transduced to express CAR, and readily expanded.

### BRIEF DESCRIPTION OF THE DRAWINGS

FIG. 1A shows a schematic diagram illustrating that the long-term functional T cell memory is a sophisticated attribute of the adaptive immune system. The stemness of cells includes 1) capacity to self-renew, 2) multipotency (can generate differentiated T cell subsets), and 3) persistence and proliferative potential. Adapted from Gattinoni et al. (2012) Nat Rev Cancer, 12(10):671-84, which is incorporated herein by reference.

FIG. 1B shows a schematic diagram of an exemplary CAR molecule. Adapted from Honikel and Olejniczak (2022) Biomolecules 12(9):1303, which is incorporated herein by reference.

FIG. 2 shows the lung cancer model of resection and metastatic recurrence. The top panel shows the complete resection of tumor, TDLN, and non-draining node. The bottom panel shows systemic metastatic recurrence after resection. TDLN (Tumor Draining Lymph Node).

FIG. 3 shows heterogeneous anti-tumor response to PD-1 inhibition.

FIG. 4 shows heterogenous TDLN T cell response to PD-1 inhibition.

FIG. 5 shows that “stem cell-like” T cells are maintained in the TDLN. Stem cell-like T cells are maintained in the lymphoid tissues. There is an antigen-specific CD8 T cell population that cell proliferates as well as differentiates into other memory population=“stem cell like.” SCM CD8 T cell subset is PD-1+ CXCR5+ and undergoes a robust response to anti-PD-1 therapy. SCM are almost exclusively found in the secondary lymphoid tissues: “lymphoid resident” T cells. There is a reservoir of SCM CD8 T cells from the lymph node that migrate to the tumor for a sustained immune response.

FIG. 6 shows that IL-15Rα-PDL-1 (KD033) preferentially increases CXCR5+ memory phenotype accumulation at TDLN.

FIG. 7 shows that IL-15Rα-PDL-1 alone and in combination with PD-1 inhibition decreases metastatic recurrence.

FIG. 8 shows that tumor draining lymph nodes in early-state NSCLC patients maintain a PD-1+ memory T-cell population.

FIG. 9 shows that tumor draining lymph nodes in early-stage NSCLC patients maintain a PD-1+ “stem cell like” memory T-cell population.

FIG. 10 shows that PD-1+ CXCR5+ CD8 T cells are found in NSCLC benign TDLN.

FIG. 11A-FIG. 11C show that the patient TDLN CD8+ T cells can be harvested and maintained ex vivo. Phenotypic and functional disparities in patient-harvested T cells. Representative data from a patient's tumor, tumor-draining lymph nodes (tdLN), and peripheral blood. (FIG. 11A) Multipanel flow cytometry reveals heightened PD-1 receptor expression and diminished IL-7RA levels in tumor-infiltrating CD8+ T cells. (FIG. 11B) Stimulation with PMA demonstrates comparable IFN-gamma and granzyme B production in peripheral blood and tdLN T cells from the same patient, as assessed by multipanel flow cytometry. (FIG. 11C) Ex vivo culture of T cells from the same patient highlights preferential expansion of tdLN and peripheral blood-derived T cells over tumor-derived T cells upon treatment with IL-7 and IL-15 on day 8.

FIG. 12 shows a schematic diagram showing an adoptive cell therapy. Adapted from Met et al. (2019) Semin Immunopathol 41(1):49-58. The left panel shows an adoptive cell therapy involving tumor infiltrating lymphocytes (TILs). The right panel shows an adoptive cell therapy involving chimeric antigen receptor (CAR) T cells.

FIG. 13 shows the comparison of the TILs therapy vs. CAR/TCR-T cell therapy.

FIG. 14 shows a schematic diagram illustrating T cells Selected from Lymph node Acquisition (TSLA). Tumor-antigen specific CXCR5+CD8+ T cells are harvested from the tumor draining lymph nodes of lung cancer patients, transduced with an ICAM-1 targeting CAR, expanded ex vivo and infused.

FIG. 15 shows the comparison of an adoptive cell therapy involving T cells selected from peripheral blood (left column) vs. an adoptice cell therapy involving T cells selected from lymph node (right column).

FIG. 16 shows that patient TDLN T cells can be effectively transduced to express CAR.

FIG. 17 shows CAR-T cell manufacturing.

FIG. 18 shows that tumor-antigen-specific CXCR5+CD8+ T cells are harvested from tumor draining lymph nodes of lung cancer patients, transduced with an ICAM-1 targeting CAR, expanded ex vivo and infused. FIG. 18 shows a schematic of T cells harvested from lymph node acquisition for CAR T cell therapy. Tumor-draining lymph nodes host a diverse population of T cells with stem cell-like characteristics, possessing the ability to recognize tumor antigens. These T cells are isolated from patients and genetically engineered using a viral vector to express a chimeric antigen receptor (CAR) targeting a specific tumor-associated surface antigen. Following genetic modification, the CAR T cells undergo ex vivo expansion before being reintroduced into the patient, facilitating targeted immunotherapy against the cancer.

FIG. 19A-FIG. 19C show that tumor draining lymph nodes maintain a reservoir of PD-1+CD8 T cell memory subsets. FIG. 19A shows that a flow cytometric analysis of TDLN displays a larger accumulation of CD8+ T cells per node as well as T cells that are PD-1+. FIG. 19B shows that TDLN maintains a larger population of CD62L− CD44+ T cells (EM) compared to NDLN and non-tumor bearing mice. FIG. 19C shows that all lymph nodes express a larger population of PD-1+ CXCR5+CD8 T cells compared to the tumor with differing expression of CD62L and CD44 compared to NDLN and non-tumor bearing mice. CD8 T cell subsets are defined as follows: CM, PD-1+CD72L+CD44+; EM, PD-1+CD62L− CD44+; SCM PD-1+ CXCR5+CD62L+CD44−.

FIG. 20A-FIG. 20B show single cell RNA sequencing of tumor matched CD8 T cells from TDLN and tumors. FIG. 20A shows that using the TCR as a molecular barcode, the paired tumor and TDLN samples were used to identify and characterize tumor-matching (TM) TDLN CD8+ T cells that had shared TCR sequences with CD8+ T cells in 344SQ tumors in mice. TDLN maintained tumor antigen-specific memory T cells not located in the tumor. FIG. 20B represents the gene signature of memory T cells and exhaustion defining cluster.

FIG. 21 shows that TCR sequencing of CD8 T cells from TDLN and tumor shows multiple tumor-antigen specific clones are found in the tumor draining lymph node. TCR as a molecular barcode paired tumor and TDLN samples to identify and characterize tumor-matching (TM) TDLN CD8+ T cells that had shared TCR sequences with CD8+ T cells in 344SQ tumors in mice.

FIG. 22A-FIG. 22B show that CXCR5+ PD-1+CD8 T cells are primarily found in the TDLN of NSCLC patients. FIG. 22A shows the flow cytometry of resected tumor, TDLN, and peripheral blood of a NSCLC patient (1 example of multiple patients). FIG. 22B shows neogenomics multiplex immunofluorescence imaging. Representative image of an FFPE 1 cm core of TDLN.

FIG. 23 shows that difference in CD8 T cell memory subset are found in TDLN of NSCLC patients compared to peripheral blood. Flow cytometry of resected TDLN and peripheral blood of a NSCLC patient (1 example of multiple patients). CM, CD62L+CD45RA− CD45RO+ CCR7+CD28+IL-7Rα+ CXCR3+CD95+; EM, CD62L− CD45RA− CD45RO+ CCR7− CD28+IL-7Rα+ CXCR3− CD95+; TE, CD62L− CD45RA+CD45RO− CCR7− CD28− IL-7Rα− CXCR3− CD95+; Naïve, CD62L+CD45RA+CD45RO− CCR7+CD28+IL-7Rα+ CXCR3− CD95−; SCM, CD62L+CD45RA+CD45RO− CCR7+CD28+IL-7Rα+ CXCR3+CD95+. CM: Central Memory. EM: Effector Memory. TE: Terminal Effector. SCM: Stem Cell-like Memory.

FIG. 24A-FIG. 24C show that patient TDLNs display unique memory populations of PD-1+ CXCR5+CD8 T cells. FIG. 24A shows resected lymph nodes of surgical patients with early-stage cancer maintain a CXCR5+PD-1+ T cell population by flow cytometry. FIG. 24B and FIG. 24C show that CXCR5+PD-1+ T cells maintain a larger “stem-cell like” memory populations compared to CXCR5− CD8 T cells in TDLN of 5 independent patients (** p<0.05).

FIG. 25A-FIG. 25C show that T cells obtained from lymph node acquisition (TSLA) can be reproducibly harvested, expanded, and transduced to express clinical grade ICAM-1 CAR. FIG. 25A shows a schematic representation of the process involving harvesting, transducing, and expanding T cells sourced from benign lymph nodes of surgical patients with early-stage cancer. FIG. 25B shows that ICAM-1 targeting CAR can be efficiently transduced into both TDLN CD4 and CD8 T cells and display distinct memory subsets compared to peripheral blood from same patient. FIG. 25C shows the reproducible transduction and expansion compared to standard of care utilizing peripheral blood. (n=different patient TDLN and peripheral blood samples; LN=tumor-draining lymph nodes (TDLN); PB=peripheral blood).

FIG. 26A-FIG. 26C show that ICAM-1 targeting TSLA-CAR (or LN-CAR) demonstrates robust tumor rejection. Five million (5e6) CAR transduced T cells derived from resected lymph node of a NSCLC patient administered to a murine model of NSCLC. Representative bioluminescence imaging (BLI) images (FIG. 26A), quantitative analysis (FIG. 26B), and overall survival data (FIG. 26C) are shown. (Median survival: 103 days vs 66 days, respectively; p=0.006) (*** p<0.005).

FIG. 27 shows a schematic illustrating the process of acquiring T cells from lymph nodes for CAR T cell therapy. (1) Mediastinal and hillar lymph nodes are routinely harvested through surgical procedures, following established protocols for lung cancer resection (2A). (2B) T cells are isolated within a closed system, allowing for cryopreservation of CD8 and CD4 T cells or direct transduction without freezing. (3) Subsequently, T cells are transduced with chimeric antigen receptor (CAR). (4) The transduced T cells undergo ex vivo expansion and undergo quality control measures. (5) Finally, the T cells are prepared for intravenous infusion.

FIG. 28A-FIG. 28C show that CD8+ Tscm T cells are predominantly localized in the tumor-draining lymph nodes (tdLN) of surgically treated (Sx) Non-Small Cell Lung Cancer (NSCLC) patients. Tumor (FIG. 28A), tdLN (FIG. 28B), and peripheral blood (FIG. 28C) samples collected on the day of surgery were stained (representative sample from one patient; repeated n=18). These T cells were identified by initially gating on singlets (FSC-H versus FSC-A), live CD3+ T cells, and lymphocytes (SSC versus FSC). Naïve-like T cells were defined as CD45RO− CCR7+CD62L+. Within these gated populations, Tscm cells express CD95, while Tnaïve cells are CD95−. Sequential gating strategy is indicated by black straight arrows. For this analysis, frozen tissues were utilized, and CD27 was used in place of CD62L for identifying Tscm cells. FSC, forward scatter; SSC, side scatter.

FIG. 29A-FIG. 29C show that CXCR5+PD1+CD8+TCF-1^{hi} tem like T cells are primarily found in the tdLN of NSCLC patients. (FIG. 29A) Flow cytometry of resected tumor, tdLN and peripheral blood of a NSCLC patient (1 example of multiple patients). (FIG. 29B) Neogenomics multiplex immunofluorescence imaging: Representative image of an FFPE 1 cm core of tdLN shows CD8+ CXCR5+ T cells are localized to the germinal B centers of lymph nodes. (FIG. 29C) TCR-CDR3 matching of resected benign tdLN, tumor and peripheral blood of early-stage patients (n=3). Average of unique expanded clones (n>3) found in the tdLN and PB shows the dominant proportion of tumor relevant clones are found in the tdLN (255 vs 36; n=3 patients).

FIG. 30A-FIG. 30B show disparity in CD8 T cell memory subsets observed in tumor-draining lymph nodes (tdLN) of Non-Small Cell Lung Cancer (NSCLC) patients compared to peripheral blood. Flow cytometric analysis of resected tdLN and peripheral blood from an NSCLC patient (representative of multiple patients). Memory subsets defined as follows: Central Memory (CM, CD62L+CD45RA− CD45RO+ CCR7+CD28+IL-7Rα+ CXCR3+CD95+); Effector Memory (EM, CD62L− CD45RA− CD45RO+ CCR7− CD28+IL-7Rα+ CXCR3− CD95+); Terminal Effector (TE, CD62L− CD45RA+CD45RO− CCR7− CD28− IL-7Rα− CXCR3− CD95+); Naïve (CD62L+CD45RA+CD45RO− CCR7+CD28+IL-7Rα+ CXCR3− CD95−); Stem Cell Memory (SCM, CD62L+CD45RA+CD45RO− CCR7+CD28+IL-7Rα+ CXCR3+CD95+). FIG. 30A shows the flow cytometry data. FIG. 30B shows the comparison of CD8 T cell memory subsets observed in tdLN of the NSCLC patients compared to peripheral blood.

FIG. 31 shows that patient tissues exhibit a notable abundance of tumor-relevant clones in lymph nodes (LN) compared to peripheral blood from the same patient. Paired single-cell (sc) RNA and T cell receptor (TCR) sequencing were conducted on samples collected from three patients, encompassing blood, tumor, and LN specimens from each individual. LN samples were amalgamated into a single tissue sample for subsequent analysis, resulting in a total of nine tissue samples. Following the exclusion of non-CD8 T cells through fluorescence-activated cell sorting (FACS) and sc sequencing techniques, a cohort of 40,974 T cells was obtained. Subsequently, TCR sequencing was employed as a clonal barcode to monitor tumor-specific clones across various tissues. Clones identified in the tumor and another tissue were classified as tumor-matched (TM). Conforming to established literature, expanded clones were defined as possessing at least 10 T cells per clone. Notably, 50% of all T cells were associated with an expanded (TM) clone. Patient one displayed 5 expanded tumor-matched clones in peripheral blood (PB) (depicted in red) compared to 45 identified in LN (depicted in orange). Patient two exhibited 3 TM clones in PB versus 37 in LN, while patient three demonstrated 2 TM clones in PB versus 41 in LN.

FIG. 32 shows the distribution of clone sizes of the top 20 largest clones within each tissue reveals a significant prevalence of tumor-relevant dominant clones in lymph nodes (LN) compared to peripheral blood from the same patient. Paired single-cell (sc) RNA and T cell receptor (TCR) sequencing were conducted on samples collected from three patients, including blood, tumor, and LN specimens from each individual. Clones identified in the tumor and another tissue were classified as tumor-matched (TM). In Patients 1-3 (BB1906, BB1927, BB1962), the top 20 tumor-relevant clones exhibited a substantial difference in expansion magnitude in the LN (depicted in blue) compared to tumor-matched clones in peripheral blood (PB) (depicted in green).

FIG. 33A-FIG. 33D show tumor-relevant clones derived from different tissues exhibit distinct transcriptional profiles. Paired single-cell (sc) RNA and T cell receptor (TCR) sequencing were performed on samples collected from three patients, including blood, tumor, and lymph node (LN) specimens from each individual. LN samples were merged into a single tissue sample for subsequent analysis, resulting in a total of nine tissue samples. Following the exclusion of non-CD8 T cells through fluorescence-activated cell sorting (FACS) and sc sequencing techniques, a cohort of 40,974 T cells was obtained. Subsequently, TCR sequencing was utilized as a clonal barcode to track tumor-specific clones across various tissues. Clones identified in the tumor and another tissue were classified as tumor-matched (TM). Consistent with established literature, expanded clones were defined as possessing at least 10 T cells per clone. (FIG. 33A) Uniform Manifold Approximation and Projection (UMAP) visualization of all (TM) tissue samples using Seurat (FIG. 33A), and creation of a DotPlot illustrating transcription factors describing CD8 T cell state (FIG. 33B). Further analysis of UMAP was conducted to ascertain cluster function, specifically assessing transcriptional scores indicative of cytotoxic-like behavior (FIG. 33C, left) and naive central memory T cells (FIG. 33C, right). UMAP was generated for each individual tissue (FIG. 33D), and molecular scores were determined per tissue in untreated patients, those with treated partial tumor response, and those with treated no response. To elucidate the effect of treatment and response on (TM) clonal phenotype, a UMAP of all samples and T cells was generated, integrated based on patients. A total of 14 clusters were identified: clusters 1, 4, and 13 represented differentiated and effector-like phenotypes; clusters 5, 6, 7, and 9 exhibited a cytotoxic effector and effector-memory-like phenotype; clusters 0, 8, 10, and 11 displayed a more memory phenotype; and clusters 2 and 3 demonstrated a quiescent, naive, and central memory-like phenotype. A tissue-specific clustering pattern was observed, with tdLN T cells primarily clustered in the most naive-like clusters, blood T cells ranging from these naive-like clusters to more cytotoxic memory clusters, and tumor T cells predominantly found in the more differentiated effector-like clusters. Although (TM) T cells were distributed across numerous clusters, blood and tdLN expanded (TM) T cells were predominantly situated in differentiated clusters compared to other T cells, with fewer observed in very naive-like clusters.

FIG. 34. Murine model of NSCLC shows tumor-relevant CD8+ T cells are located in the tdLN and display a more stem like memory transcriptional profile. 344SQ flank tumors in mice were established for 30 days, after which both the tumor tissue and tdLN were excised for analysis. CD8+ T cells were isolated from these samples for TCR sequencing and single-cell RNA sequencing. Notably, tumor-associated T cells residing in the tdLN exhibited a progenitor-like phenotype, as demonstrated by our data, in contrast to their clonally matched counterparts in the tumor microenvironment, which showed an exhausted profile. This exhaustion was marked by a heightened expression of several inhibitory receptors (Inhibitory receptor score: LAG3, PDCD1, TIGIT, and TOX) compared to those in the quiescent state (Quiescent score: SELL, CCR7, TCF7, BCL-2, LCF1). These were also examined for protein expression of several markers by flow cytometry corresponding to known canonical phenotypes CD62L, CXCR5, PD-1, CD95, IL-7RA, TCF-1.

FIG. 35 shows transcriptomic analysis of CD8+ T cells via scRNA-seq. Delineation of tumor-matched populations and progenitor memory subsets from tdLNs juxtaposed with non-tdLN derived tumor-infiltrating lymphocytes within the NSCLC microenvironment. Single-cell transcriptomic profiling of CD8+ T cells in NSCLC (n=3 patients). Utilizing scRNA-seq, distinct populations were delineated from tumor-draining lymph nodes (tdLN) and matched tumors, spotlighting progenitor and stem like memory T cells present in both domains. Tumor-infiltrating lymphocytes not originating from tdLN were also analyzed. TCR-based matching enabled the identification of (TM) tdLN CD8+ T cells with congruent TCR sequences to those in the tumor. Data from three treatment-naive, surgically-resected early-stage NSCLC patients underscored the presence of unique memory signatures across clusters. Of note, clonally expanded (n>3) tdLN-derived T cells in tumors manifested diverse memory subsets in comparison to their counterparts displaying terminally exhausted T cells.

FIG. 36A-FIG. 36C show that tumor antigen-specific T cell clones in the lymph nodes (LN) exhibit characteristics of a more naive-like memory phenotype, subsequently undergoing exhaustion upon entry into the tumor microenvironment. Single-cell transcriptomics of tumor-specific CD8+ T cells from tumor-draining lymph nodes (tdLN) and tumors in an immunocompetent murine model of lung cancer, depicting a single representative clone (FIG. 36A). Pseud-time analysis (Diffusion Pseudotime—DPT) was visualized using PHATE maps (FIG. 36B). Transcript dynamics between each co-embedded sample pair are illustrated by the direction of arrowheads. The location of transcriptional signatures for the major cell states identified is indicated by markers on pseudotime visualizations (FIG. 36C). Gene expression profile samples visualized by PHATE maps demonstrate that when the clone is located in the tdLN (depicted in blue), there are high levels of ccr7, lef1, sell, and tcf7, which subsequently decrease once the T cells enter the tumor. Conversely, these T cells gain genes associated with exhaustion, such as ctla4, havcr2, lag3, pdcd1, and tigit.

FIG. 37 shows differential gene expression between CAR+ T cells derived from peripheral blood and lymph node. Example of 1 patient where T cells were derived from PB or LN tissue, then transduced with ICAM-1 targeting CAR. These cells were subsequently flow sorted for CAR+ T cells, and single-cell RNA sequencing was performed. Differential gene expression analysis revealed distinct patterns between blood-derived CAR T cells, which exhibited genes correlated with hypo-responsiveness (EGR1, DUSP2) and suppressed activation (CTG2, NR4A1), whereas LN-derived CAR T cells exhibited genes correlated with persistence (FXYD2) and cytotoxicity (NKG7, GZMB).

FIG. 38 shows that lymph node (LN) derived CAR T cells exhibit superior persistence of CD8+ T cells within lung tumors compared to peripheral blood (PB). Five NSG gamma 2 knockout mice were intravenously injected with 1e6 A549 tumor cells into the lungs, followed by intravenous injection of 1e6 CAR+ T cells into the lungs, sourced either from peripheral blood (PB) or lymph nodes (LN), or no T cells (control). The mice were sacrificed 30 days after CAR T cell injection, and lung tissues were harvested for flow cytometric analysis. Compared to PB-derived CAR T cells, those transduced from tdLN displayed elevated levels of persistence within the lung tumor microenvironment. Each data point represents an individual mouse, with LN and PB samples originating from the same patient and possessing equal CD4/CD8 ratios upon injection.

FIG. 39 shows that tumor-draining lymph node (tdLN) derived CAR T cells exhibit delayed tumor progression outside of the lungs compared to peripheral blood (PB) T cells. Five NSG gamma 2 mice were intravenously injected with 1e6 A549 tumor cells into the lungs followed by intravenous injection of 5e6 CAR+ T cells into the lungs, either peripheral blood CAR T cells (PB) or lymph node (LN) CAR T cells, or no T cells (control). Bioluminescence imaging (BLI) was conducted outside of the lungs to assess metastatic disease. Compared to PB and control groups, LN CAR T cells demonstrated a reduced metastatic tumor burden, suggesting potential advantages in trafficking and infiltration into peripheral tissues. * p<0.05*** p<0.005

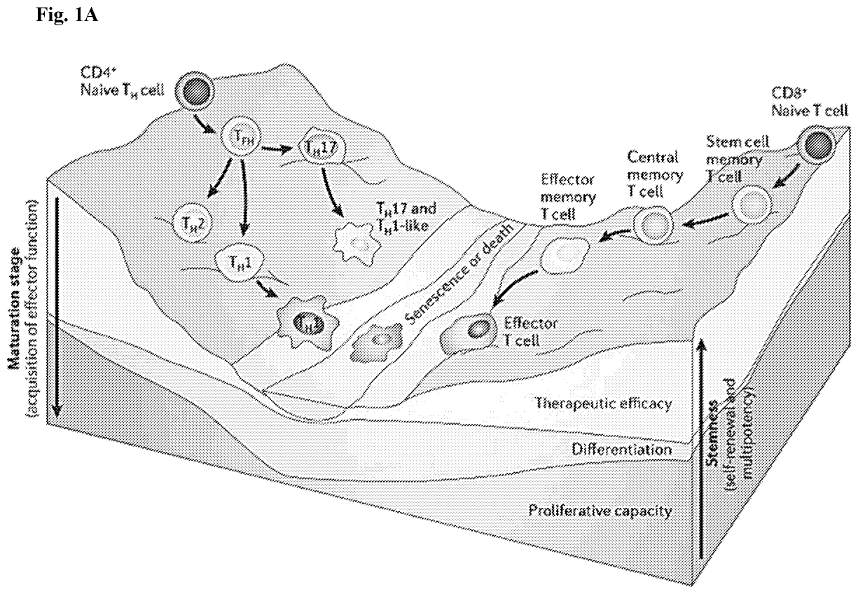

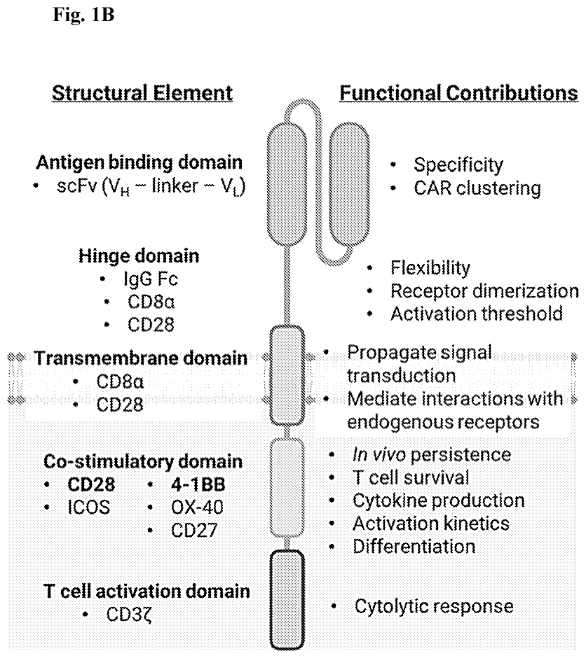

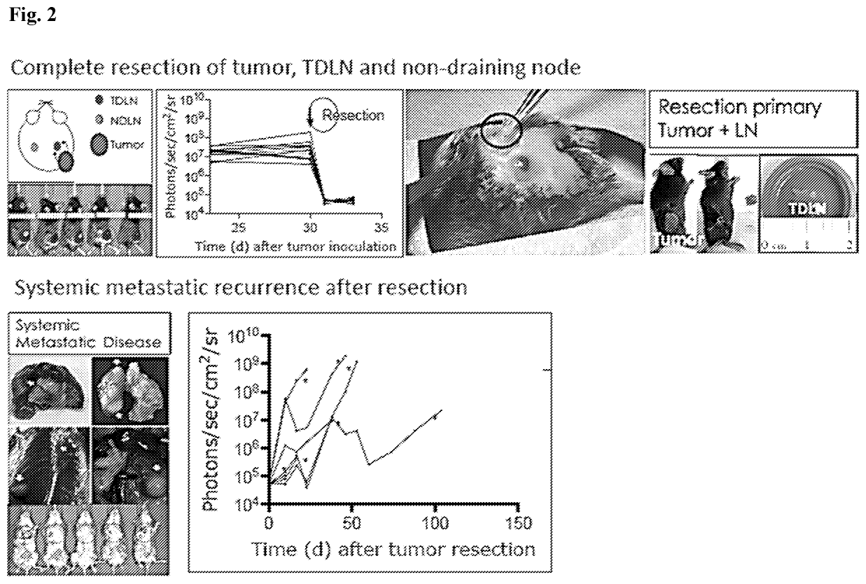

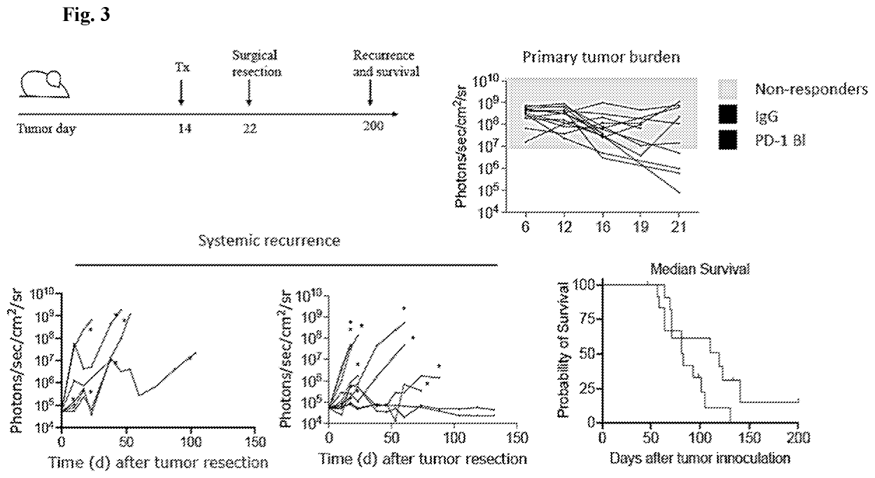

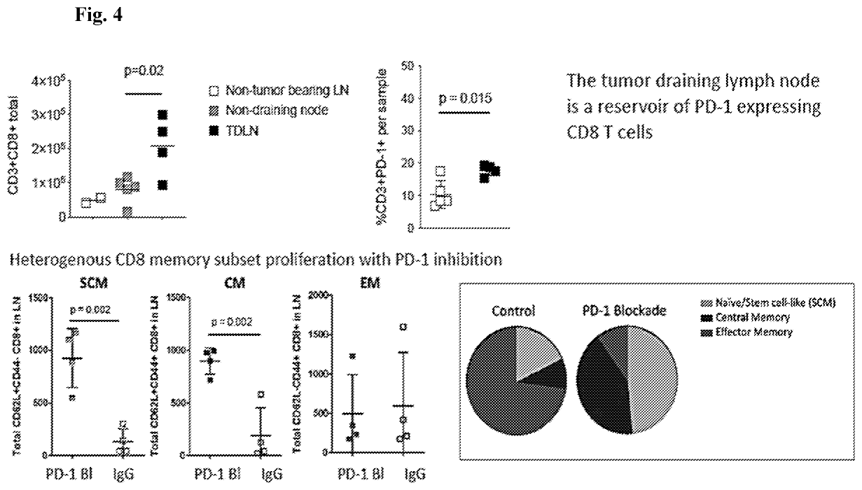

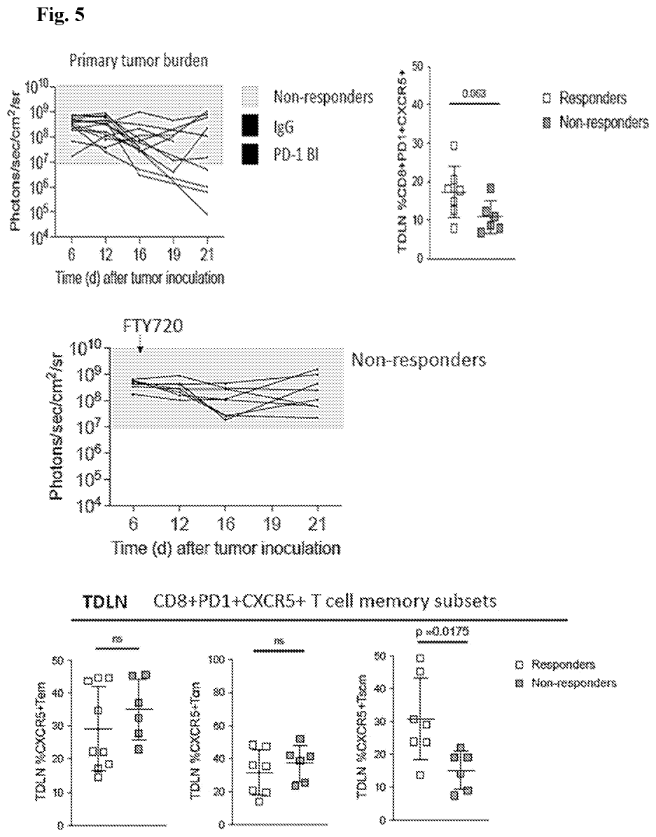

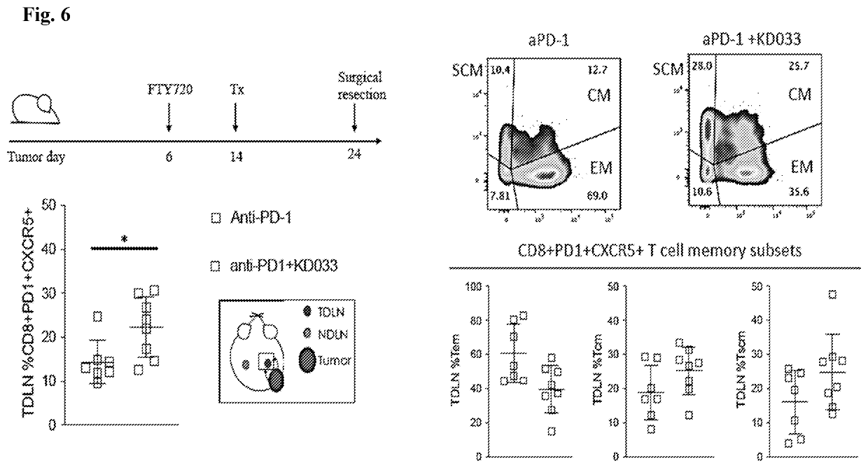

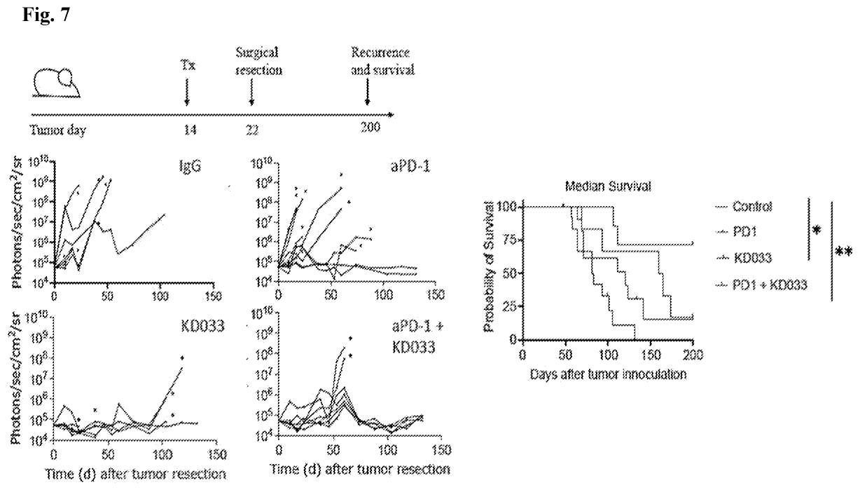

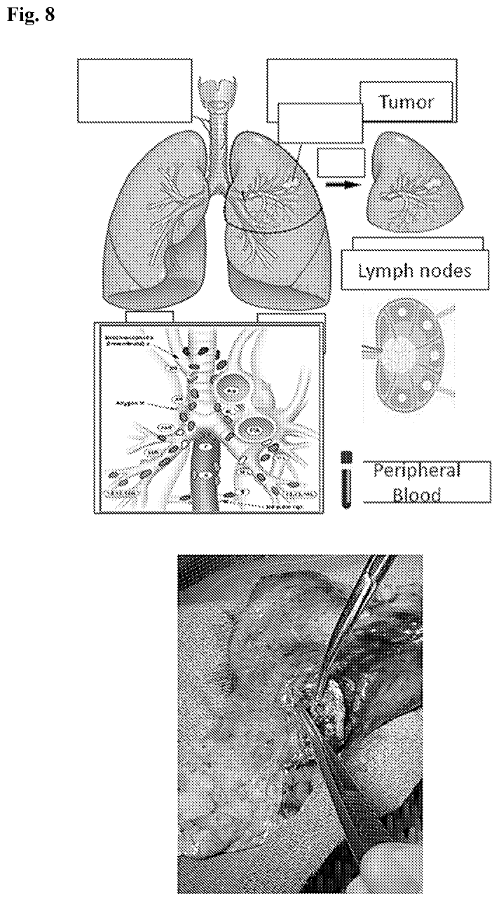

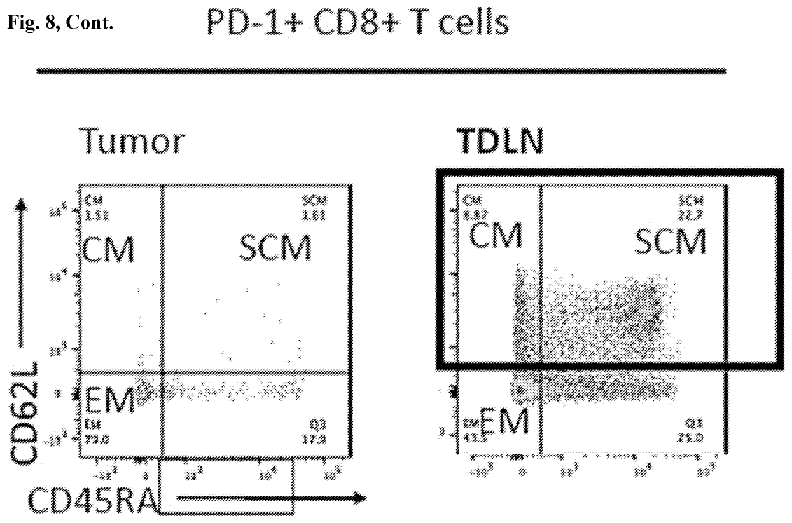

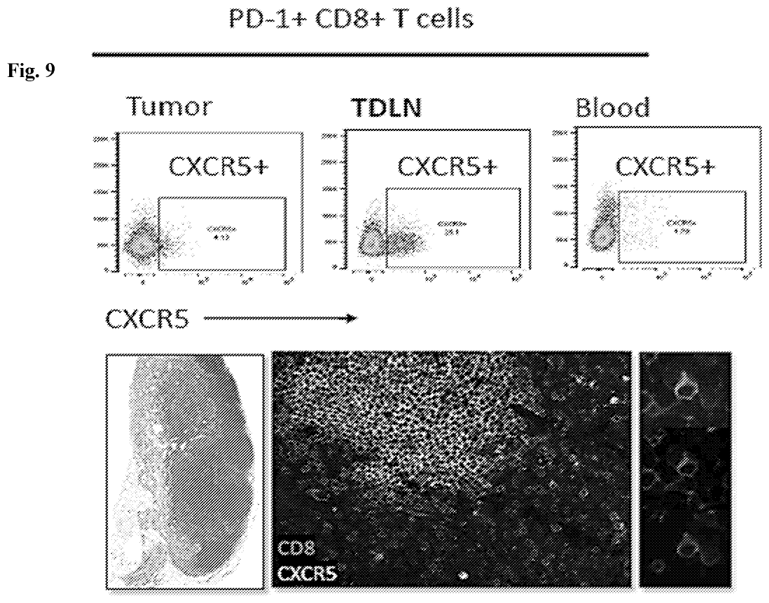

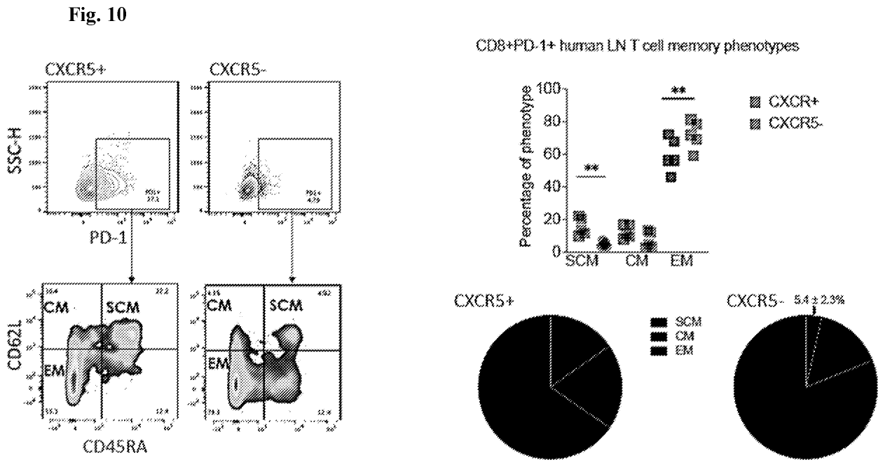

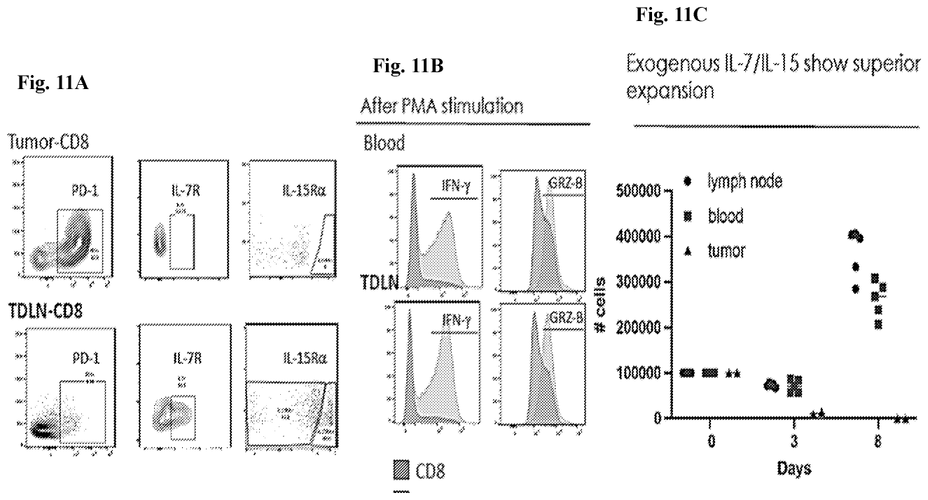

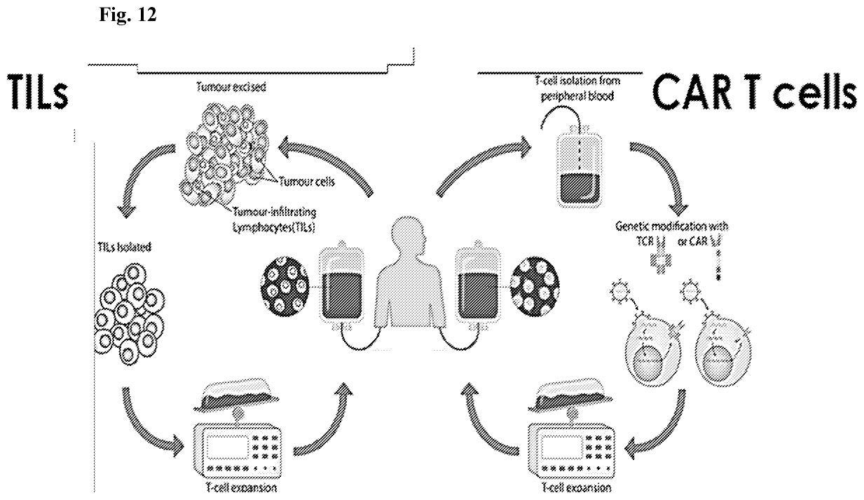

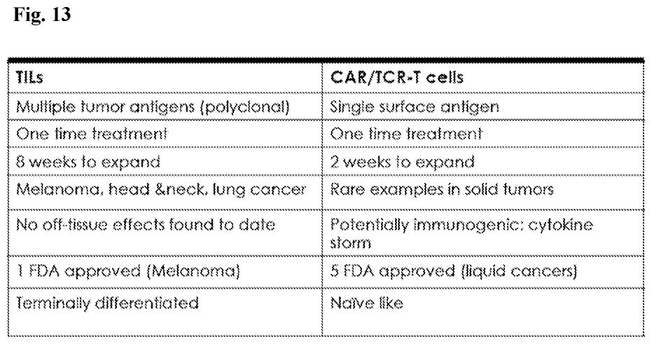

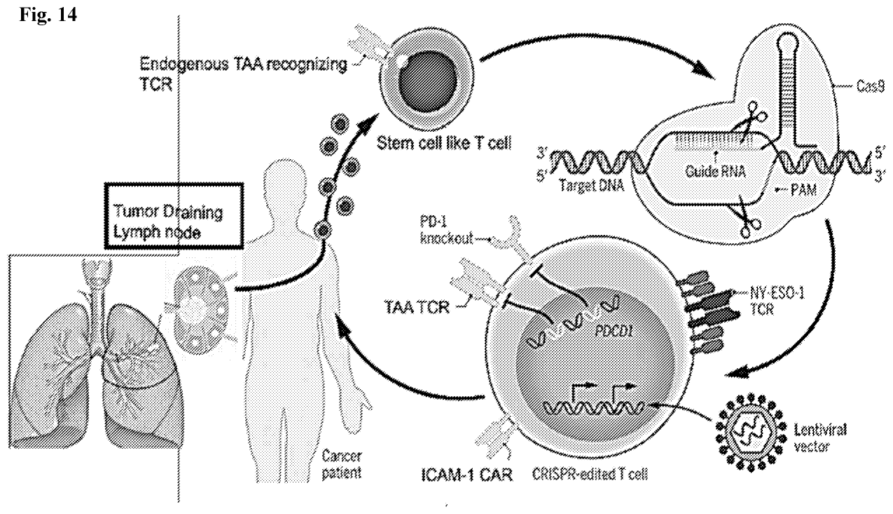

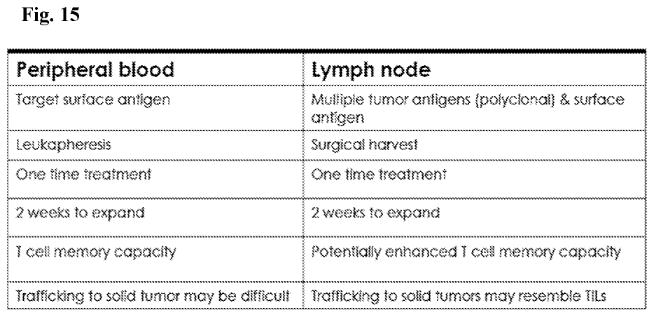

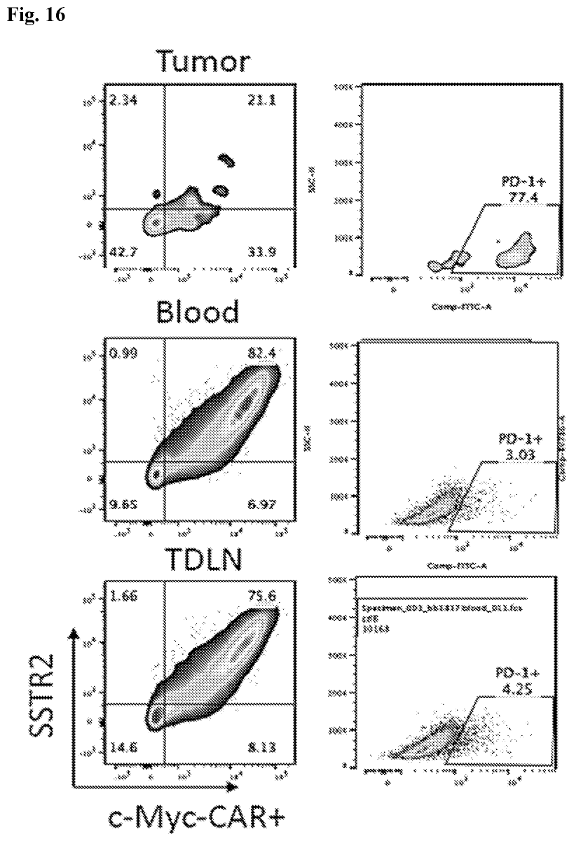

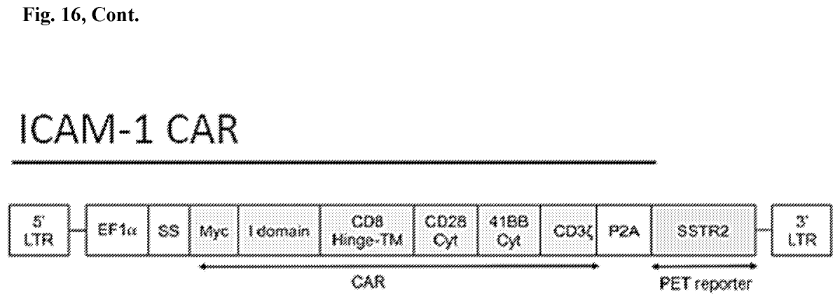

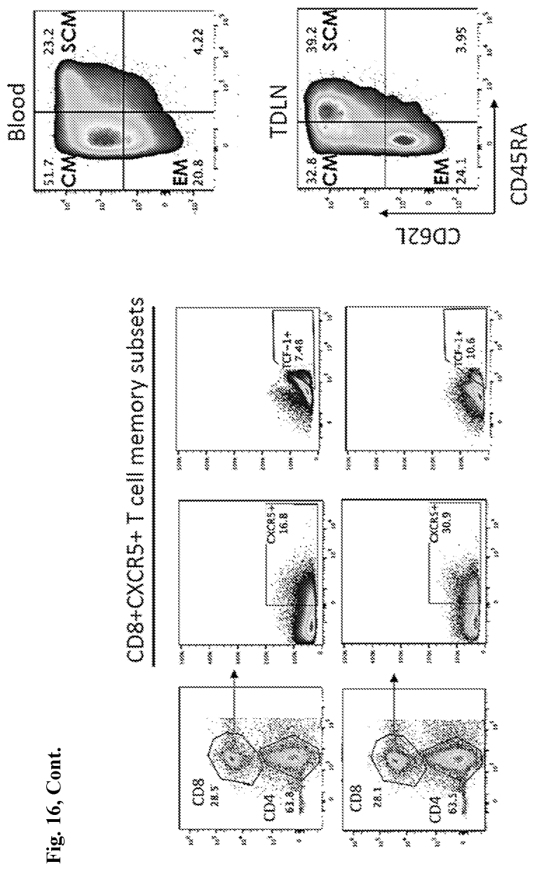

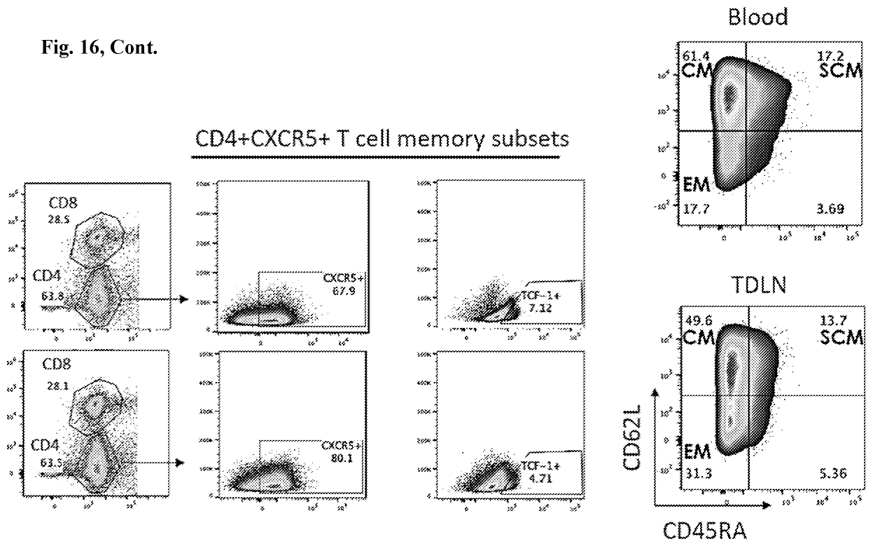

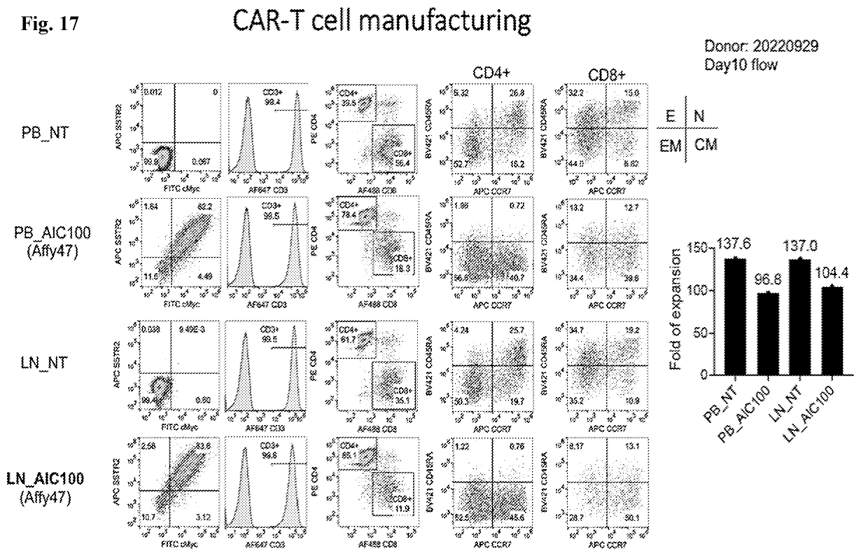

### DETAILED DESCRIPTION

Provided herein are CAR therapies comprising lymphocytes derived from TDLN and the methods of using said therapies for the treatment of cancer.

The clinical success of autologous T-cell therapies in hematologic malignancies has catalyzed their exploration in solid tumors. Current adoptive T-cell therapy (ACT) strategies bifurcate into treatments utilizing tumor-infiltrating lymphocytes (TILs) and those employing genetically modified peripheral blood T cells to express chimeric antigen receptors (CARs) redirected to attack tumors. Despite ACT's established efficacy against blood cancers, as evidenced by six FDA CAR T cell approvals and current efforts to establish them as first-line therapy, their translation to solid tumor therapy is impeded by significant immunological barriers: the selection of safe tumor-specific antigens; the complexity of solid tumor heterogeneity, deletion, or mutation of requisite antigens and subsequent ‘antigen escape’; the inefficiency of T cell trafficking to, and penetration into, solid tumor sites; and the durability and persistence of functional T cells.

To overcome these challenges, the studies presented herein pivot to exploiting tumor-draining lymph nodes (tdLNs) from non-small cell lung cancer (NSCLC) patients as a source of potent T cells. Characterization of tdLN-derived lymphocytes has revealed a population of ‘stem-like’ T cells with a broad TCR diversity, capable of responding to the heterogeneity of neoantigens present in NSCLC. Unlike TILs, these cells possess intrinsic properties for self-renewal and trans-differentiation into memory T cells, coupled with transcriptional signatures indicative of robust immune surveillance rather than exhaustion.

The methods of the present disclosure for the isolation, expansion, and genetic modification of tdLN-derived T cells have shown superb results for the production of tdLN-CAR T cells. These cells, engineered to target the overexpressed ICAM-1 surface glycoprotein in NSCLC, are manufactured to meet the quantitative and qualitative parameters of an ongoing Phase I trial targeting ICAM-1 for metastatic thyroid cancer, with a turnaround time suitable for clinical application. Our preclinical models demonstrate potent efficacy of these cells in NSCLC, supporting the rationale that tdLN-sourced CAR T cells can mediate significant tumor regression in solid tumors.

The overarching goal of this approach was to develop the novel approach of transducing lymph node-derived T cells with CAR to optimize the genetic modification of antigen-experienced stem-like T cells. Clinical use of tdLN-CARs will result in robust tumor rejection potentially translatable to multiple solid tumors, exploiting the natural endogenous immune response with cellular engineering to create a new treatment strategy.

T Cells Selected from Lymph Node Acquisition for Chimeric Antigen Receptor (CAR) Adoptive Cell Therapy for the Treatment of Cancer

The infusion of disease-targeting T cells as a therapeutic agent, has demonstrated remarkable potential to treat advanced-stage cancers. Within adoptive cell therapy (ACT), two major strategies: Chimeric antigen receptor (CAR) T cells and expansion of tumor infiltration lymphocytes (TILs) are currently in the clinic. However, barriers to ACT in solid tumors: 1) tumor antigen heterogeneity and 2) an immune inhibitory micro-environment leading to T cell exhaustion have impeded efficacy with either approach.

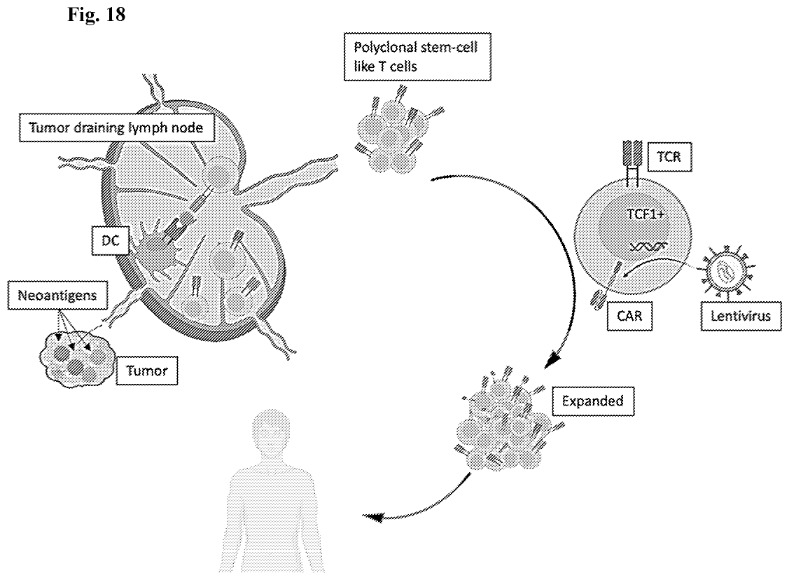

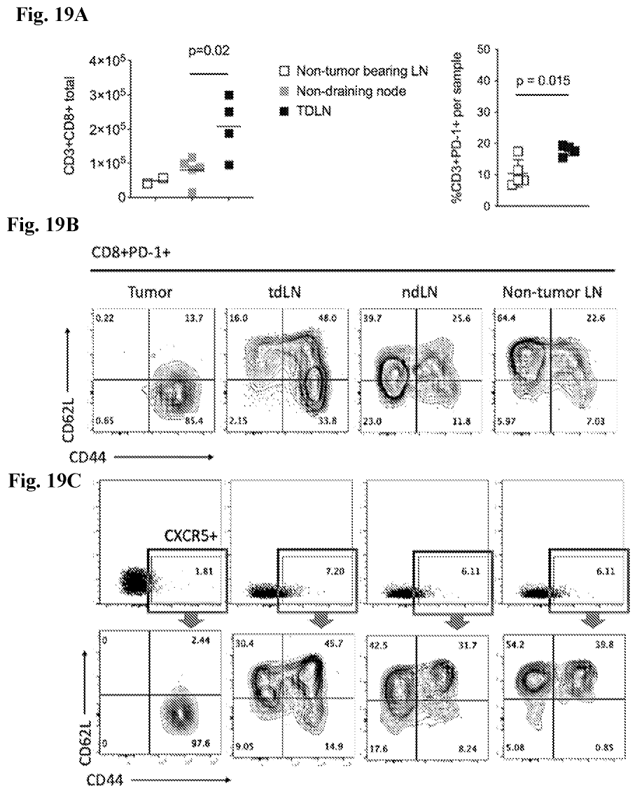

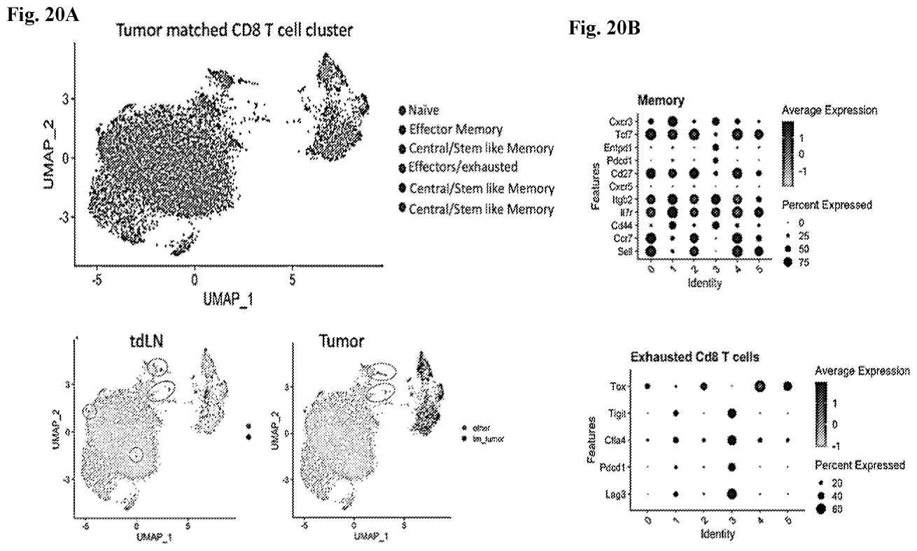

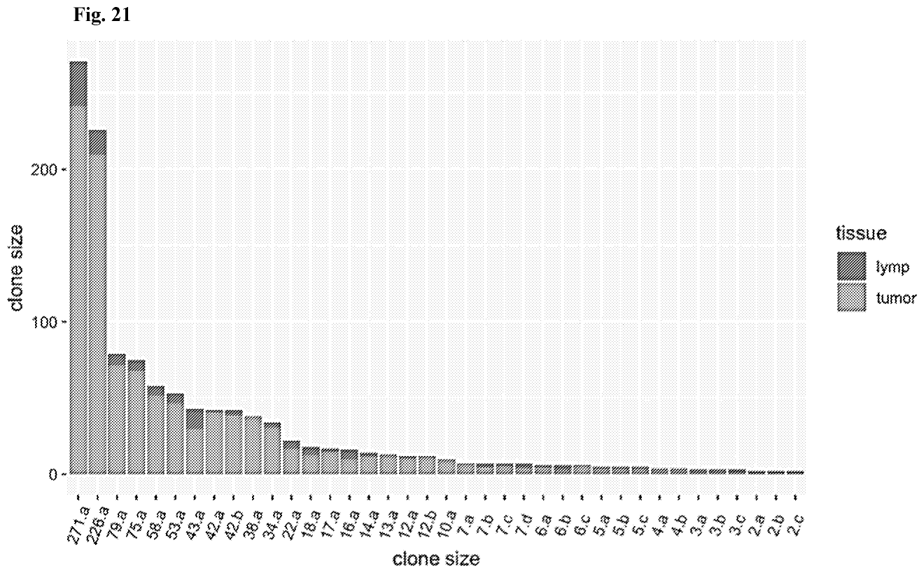

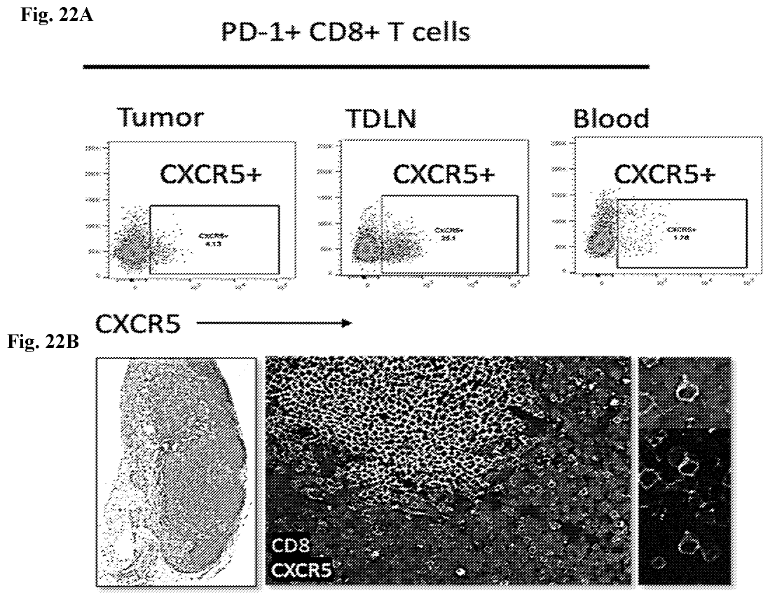

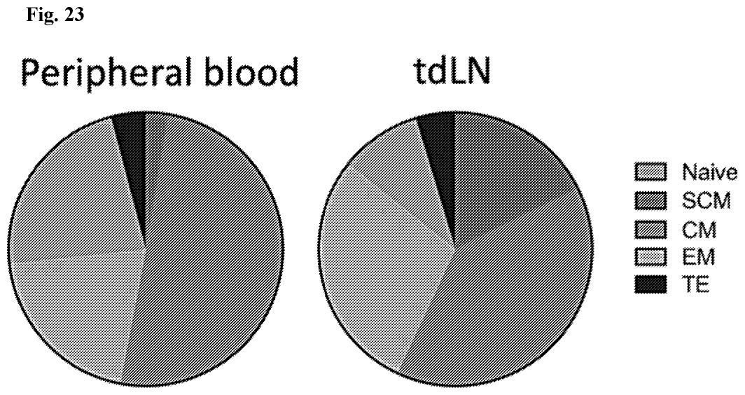

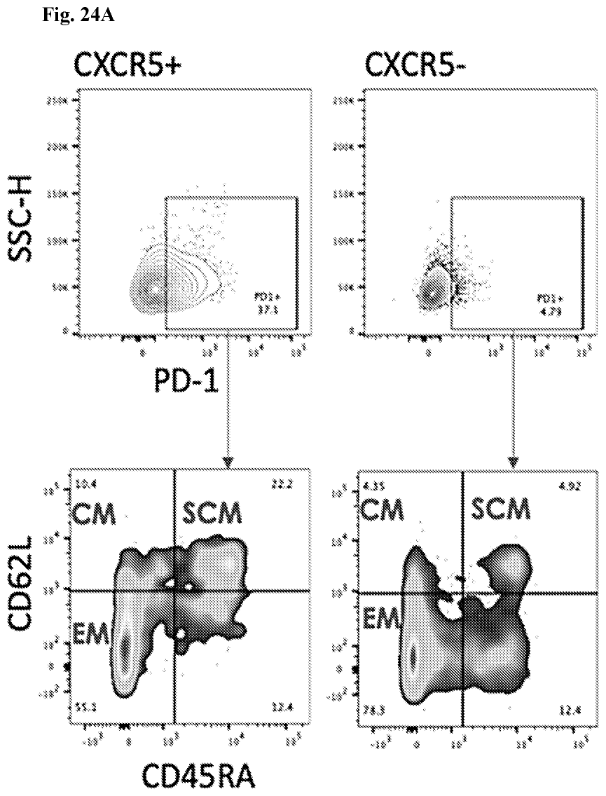

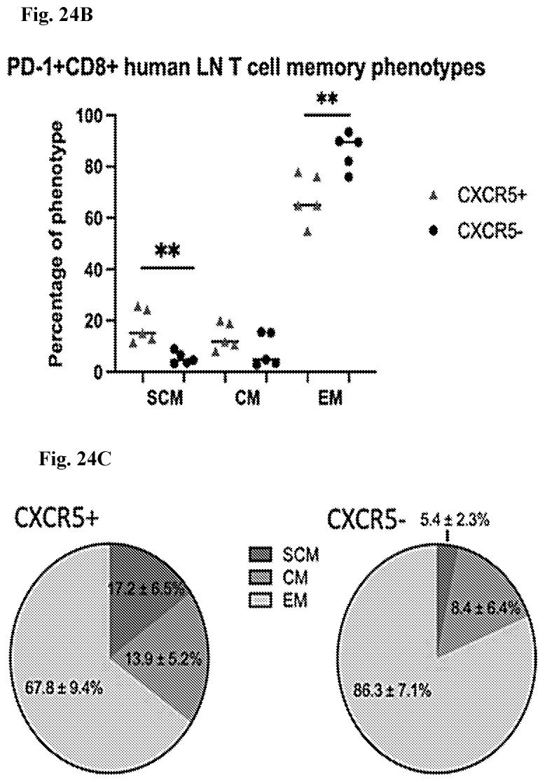

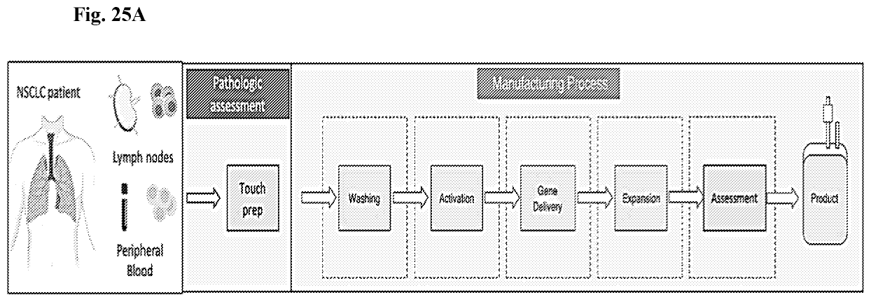

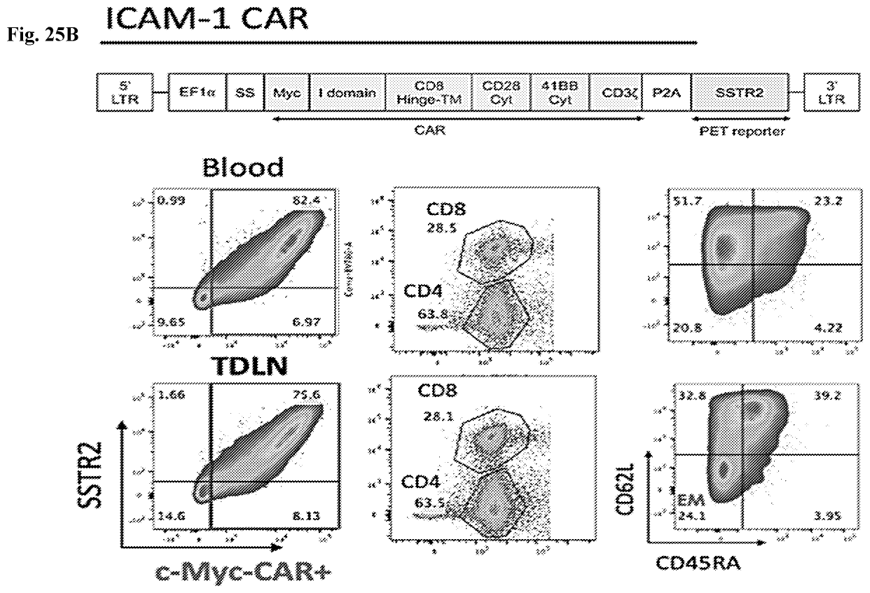

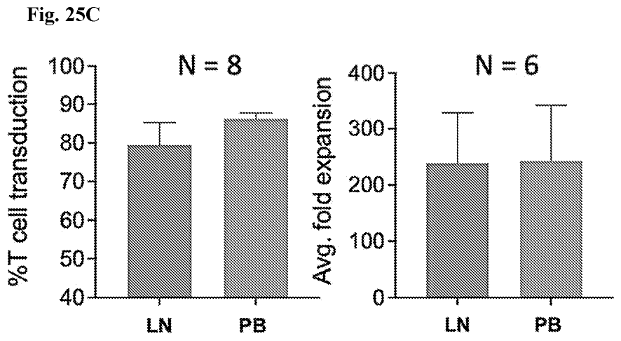

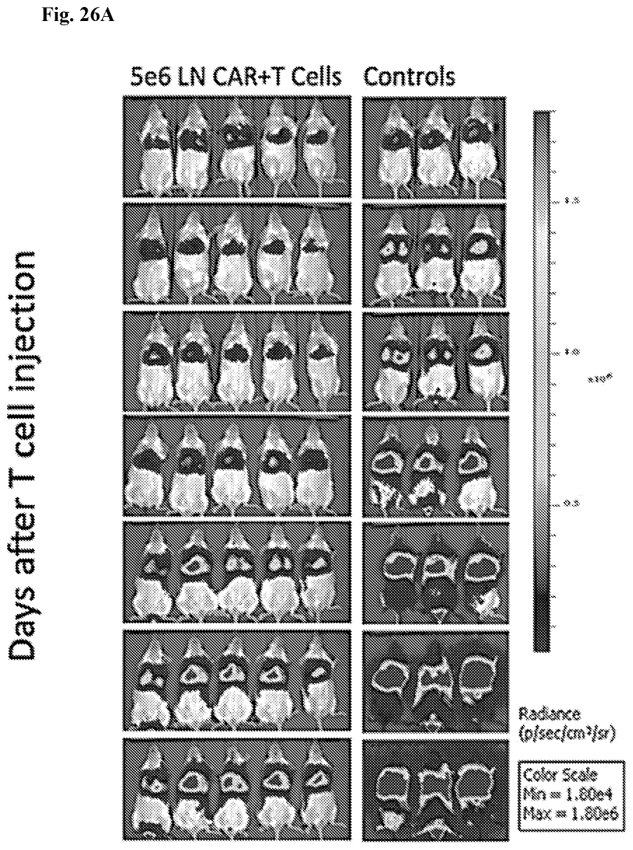

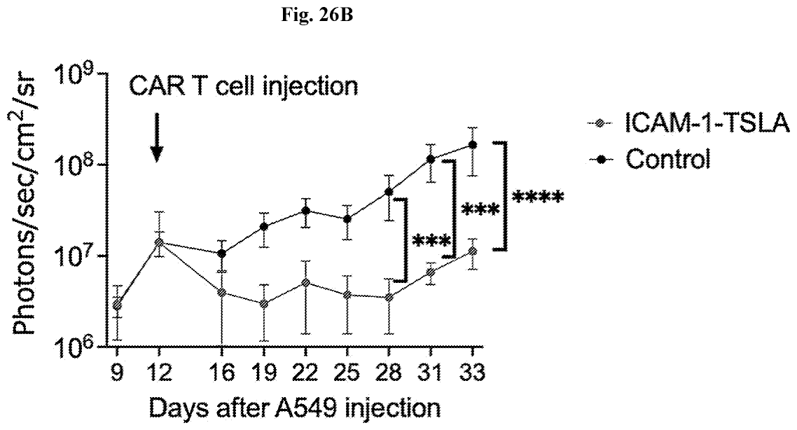

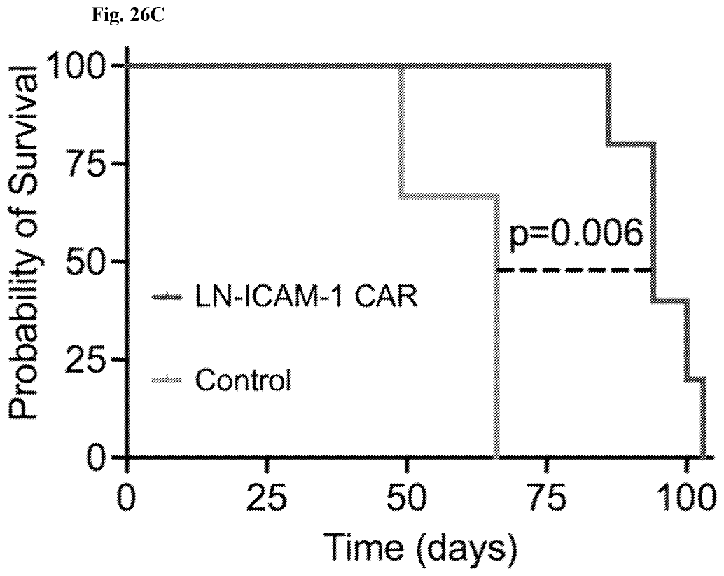

Herein, we provide a novel strategy of ACT: Utilizing T cells derived from the patient's tumor draining lymph nodes (tdLN or TDLN) as a novel form of T cell therapy (FIG. 18). The results presented herein have identified tumor antigen specific stem-cell like (SCM) memory CD8 T cell population found almost exclusively in the benign lymph nodes of non-small cell lung cancer (NSCLC) patients. In contrast to TILs where harvested T cells are thought to be terminally differentiated and show signs of T cell exhaustion and senescence, T-cells selected from lymph node acquisition (TSLA) are surprisingly 1) in a more naive, “stem cell like” state capable of persistence and T cell memory differentiation and 2) composed of polyclonal T cells capable of targeting multiple tumor antigens, therefore overcoming the solid tumor challenge of high antigen heterogeneity. Critically, we have shown that almost all patients with early and advanced NSCLC have easily accessible mediastinal and hilar adenopathy that can be reproducibly harvested, transduced to express CAR, and readily expanded.

The work provided herein define the novel approach of transducing tumor-draining lymph node derived T cells with CAR to aid in optimal genetic modification of antigen experienced stem like T cells. Using TSLA-CAR T cells will results in potent tumor elimination by converging the benefits of two robust immunotherapy strategies into a potentially transformative therapy.

### Definitions

The articles “a” and “an” are used herein to refer to one or to more than one (i.e., to at least one) of the grammatical object of the article. By way of example, “an element” means one element or more than one element.

As used herein, the term “about” when used before a numerical designation, e.g., temperature, time, amount, concentration, and such other, including a range, indicates approximations which may vary by (+) or (−) 10%, 9%, 8%, 7%, 6%, 5%, 4%, 3%, 2%, or 1%.

As used herein, the term “administering” means providing a pharmaceutical agent or composition to a subject, and includes, but is not limited to, administering by a medical professional and self-administering. Such an agent can contain, for example, a CAR T cell provided herein.

The term “binding” or “interacting” refers to an association, which may be a stable association, between two molecules, e.g., between an antigen and an antigen-binding protein (e.g., an antibody or an antigen-binding fragment thereof), e.g., between a receptor and a ligand (or a binding fragment thereof), between a peptide and a binding partner or agent, e.g., small molecule, due to, for example, electrostatic, hydrophobic, ionic and/or hydrogen-bond interactions under physiological conditions.

As used herein, the term “cancer” includes, but is not limited to, solid tumors and blood borne tumors. The term cancer includes, but is not limited to, diseases of the skin, tissues, organs, bone, cartilage, blood, and vessels, including the cervix, anus, vagina, vulva, penis, tongue base, larynx, and tonsil. The term “cancer” further encompasses primary and metastatic cancers.

The term “chimeric antigen receptor” (CAR) refers to molecules that combine a binding domain against a component present on the target cell, for example an antibody-based specificity for a desired antigen (e.g., a tumor antigen) with a T cell receptor-activating intracellular domain to generate a chimeric protein that exhibits a specific anti-target cellular immune activity. In some embodiments, CARs consist of an extracellular single chain antigen-binding domain (scFv) fused to the intracellular signaling domain of the T cell antigen receptor complex zeta chain, and have the ability, when expressed in T cells, to redirect antigen recognition based on the monoclonal antibody's specificity.

A “costimulatory domain” or “costimulatory molecule” refers to the cognate binding partner on a T-cell that specifically binds with a costimulatory ligand, thereby mediating a costimulatory response by the cell, such as, but not limited to proliferation. The costimulatory domain may be a human costimulatory domain. Exemplary costimulatory molecules include, CD28, 4-1BB, CD27, CD8, 4-1BB (CD137), OX40, CD30, CD40, PD-1, ICOS, lymphocyte function-associated antigen-1 (LFA-1), CD2, CD7, LIGHT, NKG2C, and B7-H3.

A “costimulatory ligand” refers to a molecule on an antigen presenting cell that specifically binds a cognate costimulatory molecule on a T-cell, thereby providing a signal which mediates a T cell response, including, but not limited to, proliferation activation, differentiation and the like. A costimulatory ligand can include but is not limited to CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, M1CB, HVEM, lymphotoxin beta receptor, 3/TR6, ILT3, ILT4, an agonist or antibody that binds Toll ligand receptor and a ligand that specifically binds with B7-H3.

A “costimulatory signal” refers to a signal, which in combination with a primary signal, leads to T cell proliferation and/or upregulation or downregulation of key molecules.

The term “epitope” means a protein determinant capable of specific binding to an antibody or immune cell (e.g., T cell). Epitopes usually consist of chemically active surface groupings of molecules such as amino acids or sugar side chains. Certain epitopes can be defined by a particular sequence of amino acids to which a CAR or antibody is capable of binding.

The term “gene construct” refers to a nucleic acid, such as a vector, plasmid, viral genome or the like which includes a “coding sequence” for a polypeptide or which can otherwise transcribe to a biologically active RNA (e.g., antisense, decoy, ribozyme, etc.), may be transfected into cells, e.g., mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc. In some embodiments, a gene construct may be introduced into a cell (e.g., a lymphocyte) by transfection or transduction (e.g., viral-mediated, e.g., via lentivirus or AAV).

The terms “ligand-binding domain” and “antigen-binding domain” are used interchangeably herein, and refer to that portion of a chimeric antigen receptor that binds specifically to a predetermined antigen.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The term “precancerous lesions” or “precancerous condition” refers to atypical cells and/or tissues that are associated with an increased risk of cancer. The term “precancerous lesions” may refer, for example, to dysplasia, benign neoplasia, or carcinoma in situ.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

A “signal transducing domain” or “signaling domain” of a CAR, as used herein, is responsible for intracellular signaling following the binding of an extracellular ligand binding domain to the target resulting in the activation of the immune cell and immune response. In other words, the signal transducing domain is responsible for the activation of at least one of the normal effector functions of the immune cell in which the CAR is expressed. For example, the effector function of a T cell can be a cytolytic activity or helper activity including the secretion of cytokines. Thus, the term “signal transducing domain” refers to the portion of a protein which transduces the effector function signal and directs the cell to perform a specialized function. Examples of signal transducing domains for use in a CAR can be the cytoplasmic sequences of the T cell receptor and co-receptors that act in concert to initiate signal transduction following antigen receptor engagement, as well as any derivate or variant of these sequences and any synthetic sequence that has the same functional capability. In some cases, signaling domains comprise two distinct classes of cytoplasmic signaling sequences, those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Exemplary ITAMs include those derived from TCRζ, FcRγ, FcRβ, FcRε, CD3γ, CD3δ, CD3ε, CD5, CD22, CD79a, CD79b and CD66d.

A “spacer” as used herein refers to a peptide that joins the proteins (e.g., those in a fusion protein). Generally, a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds” or “specific binding”, as used herein, when referring to a polypeptide (including CAR polypeptides) refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule. For example, in the case of the ability of a CAR to bind to a peptide presented on an MHC (e.g., class I MHC or class II MHC); typically, a CAR specifically binds to its peptide/MHC with an affinity of at least a KD of about 10′ M or less, and binds to the predetermined antigen/binding partner with an affinity (as expressed by KD) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy. In some embodiments, the subject is a mammal. In some embodiments, the subject includes a dog, a cat, a rabbit, a mouse, or a rat.

The terms “transformation”, “transfection”, or “transduction” mean the introduction of a nucleic acid, e.g., an expression vector, into a recipient cell (e.g., a mammalian cell) including introduction of a nucleic acid to the chromosomal DNA of said cell.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, to which the nucleic acid has been linked, and may or may not be able to replicate autonomously or integrate into a chromosome of a host cell. Such vectors may include any vector, (e.g., a plasmid, cosmid or phage chromosome) containing a gene construct in a form suitable for expression by a cell (e.g., linked to a transcriptional control element).

In certain embodiments, agents may be used alone or conjointly administered with another type of therapeutic agent. As used herein, the phrase “conjoint administration” or “administered conjointly” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, about a week, or more than a week of one another. Thus, a subject who receives such treatment can benefit from sa combined effect of different therapeutic agents.

### Chimeric Antigen Receptors (CARs)

Chimeric antigen receptors (CARs) are transmembrane proteins that have been engineered to give the cells (e.g., T cells, macrophages, NK cells) the new ability to target/bind a specific protein. The receptors are chimeric because they combine both antigen-binding and certain cellular functions (e.g., T cell activating function) into a single receptor. For example, the receptor can comprise an extracellular antigen-binding domain (e.g., scFv) that binds to a specific antigen (e.g., those highly and specifically expressed on the surface of cancer cells) fused to a transmembrane domain and an intracellular costimulatory domain/activation domain.

CAR polypeptides may comprise synthetic binding moieties, typically an antibody-derived single chain fragment variable (svFv) or any native antigen-sensing element, fused to intracellular signaling domains composed of the TCR zeta chain and costimulatory molecules such as CD28 and/or 4-1BB. The advantages of CAR mediated targeting include: 1) the provision of activation, proliferation, and survival signals in-cis via a single binding event, compared to the natural, non-integrated TCR and costimulatory signaling; 2) the ability to bypass the downregulation of MHC by tumor cells through MHC-independent antigen recognition; and 3) a reduced activation threshold as well as recognition of tumor cells with low antigen density enabled by the high affinity interaction between CAR and antigen.

### CAR T Therapy

Chimeric antigen receptor T cells (CAR T cells) are T cells that are engineered to express the CAR proteins for cancer therapy. CARs enable T cells to recognize tumor-associated antigens (TAAs) in a major histocompatibility complex (MHC)-independent manner. CAR T therapy can use T cells that are autologous or allogeneic to the patient. After CAR T cells are infused into a patient, they act as a “living drug” against cancer cells. When they come in contact with their targeted antigen on a cell, CAR T cells bind to it and become activated, then proceed to proliferate and become cytotoxic. CAR T cells destroy cells through several mechanisms, including extensive stimulated cell proliferation, increasing the degree to which they are toxic to other living cells (cytotoxicity) and by causing the increased secretion of factors that can affect other cells such as cytokines, interleukins and growth factors. The first CAR T cell therapies were FDA-approved in 2017, and there are now 6 approved CAR T therapies.

There are several variations/generations of CAR designs. The first reports of tumor-targeting CARs demonstrated that an scFv recognizing antigens such as human epidermal growth factor receptor 2 (HER2) fused to the CD3ζ signaling domain can elicit tumor-specific cytotoxicity, but T cells expressing these “first-generation” CARs that included only the CD3ζ chain for T-cell signaling generally failed to elicit potent antitumor effects. In the following years, second- and third-generation CARs emerged that included one or two costimulatory domains, respectively, drawing from the biological understanding that the endogenous TCR requires association with other costimulatory or accessory molecules for robust signaling. Most commonly derived from CD28 or 4-1BB, these costimulatory domains conferred more potent antitumor cytotoxicity, increased cytokine production, and improved proliferation and persistence of CAR-T cells. The choice of costimulatory domain has an impact on a wide range of properties, including metabolic pathways, T-cell memory development, and antigen-independent tonic signaling, prompting further research into other costimulatory domains. For example, a third-generation CAR with OX40 and CD28 costimulatory domains repressed CD28-induced secretion of interleukin (IL)-10, an anti-inflammatory cytokine that compromises T-cell activity. In addition, the inducible T-cell (ICOS) costimulatory domain in combination with either CD28 or 4-1BB costimulation increased in vivo persistence, and MyD88/CD40 costimulation improved in vivo proliferation of CAR-T cells. More recently, fourth-generation CARs that incorporate additional stimulatory domains, commonly referred to as “armored” CARs, have been reported. In one example, the engineered armored CAR-T cells termed “T cells redirected for universal cytokine-mediated killing” (TRUCK) have been engineered to secrete the proinflammatory cytokine IL-12 to stimulate innate immune cells against the tumor and resist inhibitory elements of the TME, including regulatory T (Treg) cells and myeloid-derived suppressor cells (MDSCs). The secretion of other soluble factors has been studied, including IL-15 or IL-18 to enhance T cell proliferation, as well as the combination of CCL19 and IL-7 to recruit endogenous immune cells and establish a memory response against tumors.

The compositions and methods of the present disclosure may utilize any known CAR design known in the art (e.g., for example, the CAR design described in WO2018044534A1, WO2021211510A2, WO2022126084A1, WO2007070488A3, each of which is incorporated herein by reference.

In some embodiments, a CAR polypeptide comprises at least one costimulatory region comprising a cluster of differentiation 28 (CD28) domain. In some embodiments, a CAR polypeptide comprises at least one costimulatory region comprising a 4-1BB domain. In some embodiments, a CAR polypeptide comprises at least one costimulatory region comprising both CD28 domain and 4-1BB domain. Additionally, the hinge/spacer region and/or the transmembrane region of the CAR or the transmembrane region of the CAR may comprise a CD28 domain. In some embodiments, a CAR polypeptide comprises an amino acid sequence with at least 50%, at least 60%, at least 70%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to an amino acid sequence set forth in Table 1. In some embodiments, a CAR polypeptide comprises a fragment (e.g., a functional fragment) of a polypeptide whose exemplary sequence is shown in Table 1. Fragments and variations of the sequences shown in Table 1 have been used to make a functional CAR polypeptide and are well known in the art.

In certain embodiments, the binding domain and/or extracellular domain of a CAR provided herein provides the CAR with the ability to bind to the target antigen of interest. A binding domain (e.g., a ligand-binding domain or antigen-binding domain) can be any protein, polypeptide, oligopeptide, or peptide that possesses the ability to specifically recognize and bind to a biological molecule (e.g., a cell surface receptor or tumor protein, or a component thereof). A binding domain includes any naturally occurring, synthetic, semi-synthetic, or recombinantly produced binding partner for a biological molecule of interest. For example, and as further described herein, a binding domain may be antibody light chain and heavy chain variable regions, or the light and heavy chain variable regions can be joined together in a single chain and in either orientation (e.g., VL-VH or VH-VL). A variety of assays are known for identifying binding domains of the present disclosure that specifically bind with a particular target, including Western blot, ELISA, flow cytometry, or surface plasmon resonance analysis (e.g., using BIACORE analysis). The target may be an antigen of clinical interest against which it would be desirable to trigger an effector immune response that results in tumor killing.

In some embodiments, CAR binds a cancer antigen expressed on the surface of a cancer cell or a tumor. In some embodiments, the cancer antigen is ICAM-1 (also called CD54). ICAM-1 gene encodes a cell surface glycoprotein which is typically expressed on endothelial cells and cells of the immune system. It binds to integrins of type CD11a/CD18, or CD11b/CD18 and is also exploited by Rhinovirus as a receptor. ICAM-1 is a transmembrane protein possessing an amino-terminus extracellular domain, a single transmembrane domain, and a carboxy-terminus cytoplasmic domain. The structure of ICAM-1 is characterized by heavy glycosylation, and the protein's extracellular domain is composed of multiple loops created by disulfide bridges within the protein. The dominant secondary structure of the protein is the beta sheet, leading researchers to hypothesize the presence of dimerization domains within ICAM-1. ICAM-1 is a type of intercellular adhesion molecule continuously present in low concentrations in the membranes of leukocytes and endothelial cells. Upon cytokine stimulation, the concentrations greatly increase. ICAM-1 can be induced by interleukin-1 (IL-1) and tumor necrosis factor (TNF) and is expressed by the vascular endothelium, macrophages, and lymphocytes. ICAM-1 is a ligand for LFA-1 (integrin), a receptor found on leukocytes. When activated, leukocytes bind to endothelial cells via ICAM-1/LFA-1 and then transmigrate into tissues.

“Intercellular adhesion molecule-1” or “ICAM-1,” i.e. GenBank Accession Nos. NM_000201, NP_000192, is the ligand for αLβ2 integrin, and its N-terminal domain (D1) binds to the αL1 domain through the coordination of ICAM-1 residue Glu-34 to the MIDAS metal. ICAM-1 is typically expressed on endothelial cells and cells of the immune system. ICAM-1 binds to integrins of type αLβ2 and αMβ2. ICAM-1 is upregulated in several carcinomas and the associated stroma as well as in inflammatory conditions. Aside from diseased tissues, ICAM-1 is basally expressed in several cell types including endothelial cells, immune cells, and some epithelial cells.

“Lymphocyte function-associated antigen-1,” “LFA-1,” “αLβ2 integrin,” or “CD18/CD11a” refers to a member of the leukocyte integrin subfamily. LFA-1 is found on all T cells and also on B cells, macrophages, neutrophils, and NK cells, and is involved in recruitment to the site of infection. It binds to ICAM-1 on antigen-presenting cells and functions as an adhesion molecule.

As used herein, “I domain” refers to the I domain of the αL subunit of LFA-1, and is an allosteric mediator of ligand binding to LFA-1. The I domain is a native ligand of ICAM-1. The ligand binding site of the I domain, known as a metal ion-dependent adhesion site (MIDAS), exists as two distinct conformations allosterically regulated by the C-terminal α7 helix. A wild-type (WT) I domain encompasses amino acid residues 130-310 of the 1145 amino acid long mature αL integrin subunit protein (SEQ ID NO: 6, which is the amino acid residues 26-1170 of GenBank Accession No. NP_002200).

Additional details of the I domain or an exemplary CAR polypeptide comprising the I domain are disclosed in Patent Publication No. WO2018052594A1, which is incorporated herein by reference.

In some embodiments, the CAR polypeptide comprises a polypeptide that binds ICAM-1. In some embodiments, the polypeptide that binds ICAM-1 comprises a fragment of an antibody (e.g., ScFv). Single chain antibodies may be cloned from the V region genes of a hybridoma specific for a desired target. A technique which can be used for cloning the variable region heavy chain (VH) and variable region light chain (VL) has been described, for example, in Orlandi et al., PNAS, 1989; 86: 3833-3837, which is incorporated herein by reference. Thus, in certain embodiments, a binding domain comprises an antibody-derived binding domain but can be a non-antibody derived binding domain. An antibody-derived binding domain can be a fragment of an antibody or a genetically engineered product of one or more fragments of the antibody, which fragment is involved in binding with the antigen.

Various antibodies that bind ICAM-1 are commercially available. For example, BioLegend (San Diego, CA) carries the ICAM-1-binding antibody with the following catalog numbers: CD54 Antibody (322706), CD54 Antibody (322708), CD54 Antibody (322702), CD54 Antibody (322712), CD54 Antibody (322713), CD54 Antibody (322707), CD54 Antibody (322714), CD54 Antibody (322718), CD54 Antibody (322715), CD54 Antibody (322716), CD54 Antibody (322720), CD54 Antibody (353107), CD54 Antibody (353106), CD54 Antibody (353110), CD54 Antibody (353108), CD54 Antibody (353109), CD54 Antibody (353105), CD54 Antibody (353101), CD54 Antibody (353102), CD54 Antibody (353125), CD54 Antibody (353126), CD54 Antibody (353129), CD54 Antibody (322722), CD54 Antibody (353132), CD54 Antibody (353131), CD54 Antibody (353130), CD54 Antibody (322721), CD54 Antibody (353133), and CD54 Antibody (353134). OriGene (Rockville, MD) carries the ICAM-1-binding antibody with the following catalog numbers: AM03205AC-N, AM03205AF-N, AM03205BT-N, AM03205FC-N, AM03205PP-N, AM03205RP-N, AM06428SU-N, AM08311PU-N, AM08420PU-N, AM26247BT-N, AM26247PU-N, AM31187AF-N, AM31187FC-N, AM31187PU-N, AM31187RP-N, AP01342PU-N, AP01608PU-N, AP02381PU-N, AP02381PU-S, AP02637PU-N, AP02637PU-S, AP26345PU-N, AP26388BT-N, BM2448P, BM2448PE, BM2449P, BM4050, BM4050B, CF506861, CF506870, DDX0150P-100, DDX0151A488-100, DDX0151A546-100, DDX0151A647-100, DDX0151P-100, SM010A, SM1138F, SM1138P, SM1138PT, SM1156F, SM1156FT, SM1156LE, SM1156P, SM1156PS, SM1156PT, SM1156R, SM286F, SM286FX, SM286LE, SM286P, SM286PS, TA309971, TA311119, TA320348, TA325547, TA325548, TA328339, TA328340, TA332780, TA333215, TA346974, TA346975, TA348959, TA349622, TA351056, TA353227L, TA354412, TA506861, TA506861AM, TA506861BM, TA506861S, TA506870, TA506870AM, TA506870BM, and TA506870S. Santa Cruz Biotechnology (Dallas, TX) carries the following ICAM-1-binding antibodies: ICAM-1 (15.2), ICAM-1 (G-5), ICAM-1 (P2A4), ICAM-1 (6.5B5), ICAM-1 (LB-2), ICAM-1 (1A29), ICAM-1 (P1W16), ICAM-1 (2Q710), ICAM-1 (28), and ICAM-1 (H-4). ThermoFisher Scientific (Waltham, MA) carries 56 ICAM-1-binding antibodies including those with the following catalog numbers: Cat #MA5407, Cat #MA5-13021, and Cat #16-0541-81.

In other embodiments, the polypeptide that binds ICAM-1 comprises a non-antibody protein that specifically binds ICAM-1, e.g., LFA-1 or a fragment thereof. In some such embodiments, the polypeptide that binds ICAM-1 comprises an “I domain” of LFA-1 or a fragment thereof. In some embodiments, the I domain comprises the amino acid sequence set forth in Table 1.

TABLE 1
Exemplary Amino Acid Sequences

SEQ ID NO: 1 Amino Acid Sequence of Human CD28 protein (UniProt P10747)

| | |
|---|---|
| 1 | MLRLLLALNL FPSIQVTGNK ILVKQSPMLV AYDNAVNLSC KYSYNLFSRE |
| 51 | FRASLHKGLD SAVEVCVVYG NYSQQLQVYS KTGFNCDGKL GNESVTFYLQ |
| 101 | NLYVNQTDIY FCKIEVMYPP PYLDNEKSNG TIIHVKGKHL CPSPLFPGPS |
| 151 | KPFWVLVVVG GVLACYSLLV TVAFIIFWVR SKRSRLLHSD YMNMTPRRPG |
| 201 | PTRKHYQPYA PPRDFAAYRS |

The cytoplasmic sequence of CD28 (amino acid residues 180-220 of SEQ ID NO:1 according to UniProt) is a highly conserved sequence that comprises the costimulatory domain that can be included in the CAR polypeptides of the present disclosure. The cytoplasmic portion of CD28 contains a critical motif called the PYAP motif, which is essential for the recruitment and activation of downstream signaling molecules like PI3K and Grb2. The sequence of the PYAP motif is Proline-Tyrosine-Alanine-Proline (P-Y-A-P).

| Amino Acid Sequence of Human 4-1BB (UniProt Q07011) | |
|---|---|
| SEQ ID NO: 2 | |
| 1 | MGNSCYNIVA TLLLVLNFER TRSLQDPCSN CPAGTFCDNN RNQICSPCPP |
| 51 | NSFSSAGGQR TCDICRQCKG VFRTRKECSS TSNAECDCTP GFHCLGAGCS |
| 101 | MCEQDCKQGQ ELTKKGCKDC CFGTFNDQKR GICRPWTNCS LDGKSVLVNG |
| 151 | TKERDVVCGP SPADLSPGAS SVTPPAPARE PGHSPQIISF FLALTSTALL |
| 201 | FLLFFLTLRF SVVKRGRKKL LYIFKQPFMR PVQTTQEEDG CSCREPEEEE |
| 251 | GGCEL |
| Amino Acid Sequence of the Human CD35 Chain (UniProt P20963) | |
| SEQ ID NO: 3 | |
| 1 | MKWKALFTAA ILQAQLPITE AQSFGLLDPK LCYLLDGILF IYGVILTALF |
| 51 | LRVKFSRSAD APAYQQGQNQ LYNELNLGRR EEYDVLDKRR GRDPEMGGKP |
| 101 | QRRKNPQEGL YNELQKDKMA EAYSEIGMKG ERRRGKGHDG LYQGLSTATK |
| 151 | DTYDALHMQA LPPR |
| Amino Acid Sequence of Human CD8 transmembrane domain | |
| SEQ ID NO: 4 | |
| LLELDDYVCYASRTRKQT | |
| Amino Acid Sequence of Human ICAM-1 (Uniprot P05362) | |
| SEQ ID NO: 5 | |
| 1 | MAPSSPRPAL PALLVLLGAL FPGPGNAQTS VSPSKVILPR GGSVLVTCST |
| 51 | SCDQPKLLGI ETPLPKKELL LPGNNRKVYE LSNVQEDSQP MCYSNCPDGQ |
| 101 | STAKTFLTVY WTPERVELAP LPSWQPVGKN LTLRCQVEGG APRANLTVVL |
| 151 | LRGEKELKRE PAVGEPAEVT TTVLVRRDHH GANFSCRTEL DLRPQGLELF |
| 201 | ENTSAPYQLQ TFVLPATPPQ LVSPRVLEVD TQGTVVCSLD GLFPVSEAQV |
| 251 | HLALGDQRLN PTVTYGNDSF SAKASVSVTA EDEGTQRLTC AVILGNQSQE |
| 301 | TLQTVTIYSF PAPNVILTKP EVSEGTEVTV KCEAHPRAKV TLNGVPAQPL |
| 351 | GPRAQLLLKA TPEDNGRSFS CSATLEVAGQ LIHKNQTREL RVLYGPRLDE |
| 401 | RDCPGNWTWP ENSQQTPMCQ AWGNPLPELK CLKDGTFPLP IGESVTVTRD |
| 451 | LEGTYLCRAR STQGEVTRKV TVNVLSPRYE IVIITVVAAA VIMGTAGLST |
| 501 | YLYNRQRKIK KYRLQQAQKG TPMKPNTQAT PP |

SEQ ID NO: 6 Amino Acid Sequence of the Human integrin αL precursor (amino acid residues 26-1170 of GenBank Accession No. NP_002200 are shown below)

A wild-type (WT) I domain encompasses amino acid residues 130-310 of the 1145 amino acid long mature αL integrin subunit protein (SEQ ID NO: 6, which is the amino acid residues 26-1170 of GenBank Accession No. NP_002200).

| | |
|---|---|
| 1 | YNLDVRGARS FSPPRAGRHF GYRVLQVGNG VIVGAPGEGN STGSLYQCQS |
| 51 | GTGHCLPVTL RGSNYTSKYL GMTLATDPTD GSILACDPGL SRTCDQNTYL |
| 101 | SGLCYLFRQN LQGPMLQGRP GFQECIKGNV DLVFLFDGSM SLQPDEFQKI |
| 151 | LDFMKDVMKK LSNTSYQFAA VQFSTSYKTE FDFSDYVKWK DPDALLKHVK |
| 201 | HMLLLINTFG AINYVATEVF REELGARPDA TKVLIIITDG EATDSGNIDA |
| 251 | AKDIIRYIIG IGKHFQTKES QETLHKFASK PASEFVKILD TFEKLKDLFT |
| 301 | ELQKKIYVIE GTSKQDLTSF NMELSSSGIS ADLSRGHAVV GAVGAKDWAG |
| 351 | GFLDLKADLQ DDTFIGNEPL TPEVRAGYLG YTVTWLPSRQ KTSLLASGAP |
| 401 | RYQHMGRVLL FQEPQGGGHW SQVQTIHGTQ IGSYFGGELC GVDVDQDGET |
| 451 | ELLLIGAPLF YGEQRGGRVF IYQRRQLGFE EVSELQGDPG YPLGRFGEAI |
| 501 | TALTDINGDG LVDVAVGAPL EEQGAVYIFN GRHGGLSPQP SQRIEGTQVL |
| 551 | SGIQWFGRSI HGVKDLEGDG LADVAVGAES QMIVLSSRPV VDMVTLMSFS |
| 601 | PAEIPVHEVE CSYSTSNKMK EGVNITICFQ IKSLYPQFQG RLVANLTYTL |
| 651 | QLDGHRTRRR GLFPGGRHEL RRNIAVTTSM SCTDFSFHFP VCVQDLISPI |
| 701 | NVSLNFSLWE EEGTPRDQRA QGKDIPPILR PSLHSETWEI PFEKNCGEDK |
| 751 | KCEANLRVSF SPARSRALRL TAFASLSVEL SLSNLEEDAY WVQLDLHFPP |
| 801 | GLSFRKVEML KPHSQIPVSC EELPEESRLL SRALSCNVSS PIFKAGHSVA |
| 851 | LQMMFNTLVN SSWGDSVELH ANVTCNNEDS DLLEDNSATT IIPILYPINI |
| 901 | LIQDQEDSTL YVSFTPKGPK IHQVKHMYQV RIQPSIHDHN IPTLEAVVGV |
| 951 | PQPPSEGPIT HQWSVQMEPP VPCHYEDLER LPDAAEPCLP GALFRCPVVF |
| 1001 | RQEILVQVIG TLELVGEIEA SSMFSLCSSL SISFNSSKHF HLYGSNASLA |
| 1051 | QVVMKVDVVY EKQMLYLYVL SGIGGLLLLL LIFIVLYKVG FFKRNLKEKM |
| 1101 | EAGRGVPNGI PAEDSEQLAS GQEAGDPGCL KPLHEKDSES GGGKD |

Included in Table 1 are polypeptide molecules comprising an amino acid sequence having at least 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Table 1. Such polypeptides can have a function of the full-length polypeptide as described further herein.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA can be used to derive the polypeptide amino acid sequence, using the genetic code to translate the DNA or RNA into an amino acid sequence. Likewise, for polypeptide amino acid sequences, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

Finally, nucleic acid and amino acid sequence information encompassed by the present invention are well known in the art and readily available on publicly available databases, such as the National Center for Biotechnology Information (NCBI) or UniProt (see World Wide Web at uniprot.org).

In addition to ICAM-1, other cancer antigens may be targeted by the CAR of the present disclosure. Exemplary cancer antigens include any one or more of the following:
GD2—expressed on neuroblastoma, melanoma, and some other solid tumors.
Mesothelin—expressed on mesothelioma and pancreatic cancer.
HER2—expressed on breast cancer and some other solid tumors.
EGFR—expressed on some solid tumors, such as glioblastoma and non-small cell lung cancer.
PSMA—expressed on prostate cancer and some other solid tumors.
MUC1—expressed on a variety of solid tumors, including breast, lung, pancreatic, and ovarian cancer.
L1-CAM—expressed on a variety of solid tumors, including breast, lung, and pancreatic cancer.
CD276 (B7-H3)—expressed on a variety of solid tumors, including lung, breast, and ovarian cancer.
CD44v6—expressed on a variety of solid tumors, including pancreatic, gastric, and breast cancer.
IL13Rα2—expressed on glioblastoma and other solid tumors.
EpCAM—expressed on a variety of solid tumors, including breast, colon, and pancreatic cancer.
Fibroblast activation protein (FAP)—expressed on cancer-associated fibroblasts in many types of solid tumors.
CD133—expressed on cancer stem cells in a variety of solid tumors, including brain, colon, and pancreatic cancer.
ROR1—expressed on some solid tumors, including breast, lung, and ovarian cancer.
CD24—expressed on a variety of solid tumors, including pancreatic, gastric, and breast cancer.
B7-H4—expressed on a variety of solid tumors, including ovarian, lung, and breast cancer.
NKG2D ligands—expressed on a variety of solid tumors, including colon, prostate, and ovarian cancer.
CD47—expressed on a variety of solid tumors and plays a role in immune evasion and resistance to therapy.
GPC3—expressed on the surface of hepatocellular carcinoma (HCC) cells and some other solid tumors.
MUC1—overexpressed in many types of solid tumors, including breast, lung, pancreatic, and ovarian cancer.
Claudin 18.2—expressed on the surface of some solid tumors, including gastric and pancreatic cancer.
GD2—expressed on the surface of neuroblastoma, melanoma, and some other solid tumors.
EGFRvIII—an oncogenic variant of the epidermal growth factor receptor (EGFR) that is expressed in some solid tumors, including glioblastoma.

In certain embodiments, the CAR polypeptide of the present disclosure may comprise a linker between the various domains, added for appropriate spacing and conformation of the molecule. For example, in one embodiment, there may be a linker between the binding domain VH or VL which may be between 1-10 amino acids long. In other embodiments, the linker between any of the domains of the chimeric antigen receptor may be between 1-20 or 20 amino acids long. In this regard, the linker may be 1, 2, 3, 4, 5, 6, 7, 8, 9, 10, 11, 12, 13, 14, 15, 16, 17, 18, 19 or 20 amino acids long. In further embodiments, the linker may be 21, 22, 23, 24, 25, 26, 27, 28, 29 or 30 amino acids long. Ranges including the numbers described herein are also included herein, e.g., a linker 10-30 amino acids long.

In certain embodiments, linkers suitable for use in the CAR described herein are flexible linkers. Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)n, glycine-serine polymers, where n is an integer of at least one, glycine-alanine polymers, alanine-serine polymers, and other flexible linkers known in the art. Glycine and glycine-serine polymers are relatively unstructured, and therefore may be able to serve as a neutral tether between domains of fusion proteins such as the CARs described herein. Glycine accesses significantly more phi-psi space than even alanine, and is much less restricted than residues with longer side chains. The ordinarily skilled artisan will recognize that design of a CAR can include linkers that are all or partially flexible, such that the linker can include a flexible linker as well as one or more portions that confer less flexible structure to provide for a desired CAR structure.

The binding domain of the CAR may be followed by a “spacer,” or, “hinge,” which refers to the region that moves the antigen binding domain away from the effector cell surface to enable proper cell/cell contact, antigen binding and activation (Patel et al., Gene Therapy, 1999; 6: 412-419). The hinge region in a CAR is generally between the transmembrane (TM) and the binding domain. In certain embodiments, a hinge region is an immunoglobulin hinge region and may be a wild type immunoglobulin hinge region or an altered wild type immunoglobulin hinge region. Other exemplary hinge regions used in the CARs described herein include the hinge region derived from the extracellular regions of type 1 membrane proteins such as CD8α, CD4, CD28 and CD7, which may be wild-type hinge regions from these molecules or may be altered.

The “transmembrane” region or domain is the portion of the CAR that anchors the extracellular binding portion to the plasma membrane of the immune effector cell, and facilitates binding of the binding domain to the target antigen. In some embodiments, the transmembrane domain may be a CD3ζ transmembrane domain. Other transmembrane domains that may be employed in some embodiments include those obtained from CD8, CD8α, CD4, CD28, CD45, CD9, CD16, CD22, CD33, CD64, CD80, CD86, CD134, CD137, and CD154. In certain embodiments, the transmembrane domain is synthetic in which case it would comprise predominantly hydrophobic residues such as leucine and valine.

In certain embodiments, the CARs provided herein comprise an intracellular signaling domain. The intracellular signaling domain (also referred to herein as the “signaling domain”) comprises the part of the chimeric antigen receptor protein that participates in transducing the message of effective CAR binding to a target antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

In certain embodiments, the CARs provided herein comprise one or more immunoreceptor tyrosine-based activation motifs or ITAMs. Examples of ITAM containing primary cytoplasmic signaling sequences that are of use include those derived from TCRζ, FcRgamma, FcRβ, CD3γ, CD3δ, CD3ε, CD5, CD22, CD79a, CD79b and CD66d. In one embodiment, the intracellular signaling domain of the CARs described herein are derived from CD3ζ.

In certain embodiments, the CARs provided herein further comprise a costimulatory domain. Costimulatory molecules are cell surface molecules other than antigen receptors or Fc receptors that provide a second signal required for efficient activation and function of T lymphocytes upon binding to antigen. Examples of such co-stimulatory molecules include CD27, CD28, 4-1BB (CD137), OX40 (CD134), CD30, CD40, PD-1, ICOS (CD278), LFA-1, CD2, CD7, LIGHT, NKD2C, B7-H2 and a ligand that specifically binds CD83. Accordingly, while the present disclosure provides exemplary costimulatory domains derived from CD28. The inclusion of one or more co-stimulatory signaling domains may enhance the efficacy and expansion of T cells expressing CAR receptors. Also disclosed herein are CAR polypeptides, wherein the costimulatory region of the CAR polypeptide further comprises a 4-1BB domain (e.g., in addition to a CD28 domain). The costimulatory region of such a CAR polypeptide may comprise a complete 4-1BB domain or fragment thereof, and/or a complete CD28 domain or fragment thereof. The intracellular signaling and costimulatory signaling domains may be linked in any order in tandem to the carboxyl terminus of the transmembrane domain. Exemplary sequences of various domains are shown in Table 1.

### Nucleic Acids and Vectors

In certain aspects, also disclosed are nucleic acids and polynucleotide vectors encoding the CAR polypeptides disclosed herein.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter, and incorporating the construct into an expression vector. Typical cloning vectors contain transcription and translation terminators, initiation sequences, and promoters useful for regulation of the expression of the desired nucleic acid sequence.

In certain embodiments, the polynucleotide encoding the CAR described herein is inserted into a vector. The vector is a vehicle into which a polynucleotide encoding a protein may be covalently inserted so as to bring about the expression of that protein and/or the cloning of the polynucleotide. Such vectors may also be referred to as “expression vectors”. The isolated polynucleotide may be inserted into a vector using any suitable methods known in the art, for example, without limitation, the vector may be digested using appropriate restriction enzymes and then may be ligated with the isolated polynucleotide having matching restriction ends. Expression vectors have the ability to incorporate and express heterologous or modified nucleic acid sequences coding for at least part of a gene product capable of being transcribed in a cell. In most cases, RNA molecules are then translated into a protein. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are discussed infra. An expression vector may comprise additional elements, for example, the expression vector may have two replication systems, thus allowing it to be maintained in two organisms, for example in human cells for expression and in a prokaryotic host for cloning and amplification.

The expression vector may have the necessary 5′ upstream and 3′ downstream regulatory elements such as promoter sequences such as CMV, PGK and EF 1alpha. promoters, ribosome recognition and binding TATA box, and 3′ UTR AAUAAA transcription termination sequence for the efficient gene transcription and translation in its respective host cell. Other suitable promoters include the constitutive promoter of simian virus 40 (SV40) early promoter, mouse mammary tumor virus (MMTV), HIV LTR promoter, MoMuLV promoter, avian leukemia virus promoter, EBV immediate early promoter, and rous sarcoma virus promoter. Human gene promoters may also be used, including, but not limited to the actin promoter, the myosin promoter, the hemoglobin promoter, and the creatine kinase promoter. In certain embodiments inducible promoters are also contemplated as part of the vectors expressing chimeric antigen receptor. This provides a molecular switch capable of turning on expression of the polynucleotide sequence of interest or turning off expression. Examples of inducible promoters include, but are not limited to a metallothionine promoter, a glucocorticoid promoter, a progesterone promoter, or a tetracycline promoter.

The expression vector may have additional sequence such as 6×-histidine, c-Myc, and FLAG tags which are incorporated into the expressed CARs. Thus, the expression vector may be engineered to contain 5′ and 3′ untranslated regulatory sequences that sometimes can function as enhancer sequences, promoter regions and/or terminator sequences that can facilitate or enhance efficient transcription of the nucleic acid(s) of interest carried on the expression vector. An expression vector may also be engineered for replication and/or expression functionality (e.g., transcription and translation) in a particular cell type, cell location, or tissue type. Expression vectors may include a selectable marker for maintenance of the vector in the host or recipient cell.

In various embodiments, the vectors are plasmid, autonomously replicating sequences, and transposable elements. Additional exemplary vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). Examples of expression vectors are Lenti-X™ Bicistronic Expression System (Neo) vectors (Clontrch), pClneo vectors (Promega) for expression in mammalian cells; pLenti4/V5-DEST™, pLenti6/V5-DEST™, and pLenti6.2N5-GW/lacZ (Invitrogen) for lentivirus-mediated gene transfer and expression in mammalian cells. The coding sequences of the CARs disclosed herein can be ligated into such expression vectors for the expression of the chimeric protein in mammalian cells.

In certain embodiments, the nucleic acids encoding the CAR are provided in a viral vector. A viral vector can be that derived from, for example, a retrovirus (e.g., a foamy virus) or lentivirus. As used herein, the term, “viral vector,” refers to a nucleic acid vector construct that includes at least one element of viral origin and has the capacity to be packaged into a viral vector particle. The viral vector can contain the coding sequence for the various chimeric proteins described herein in place of nonessential viral genes. The vector and/or particle can be utilized for the purpose of transferring DNA, RNA or other nucleic acids into cells either in vitro or in vivo. Numerous forms of viral vectors are known in the art.

In certain embodiments, the viral vector containing the coding sequence for a CAR described herein is a retroviral vector or a lentiviral vector. The term “retroviral vector” refers to a vector containing structural and functional genetic elements that are primarily derived from a retrovirus. The term “lentiviral vector” refers to a vector containing structural and functional genetic elements outside the LTRs that are primarily derived from a lentivirus.

The retroviral vectors for use herein can be derived from any known retrovirus (e.g., type c retroviruses, such as Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Murine Stem Cell Virus (MSCV) and Rous Sarcoma Virus (RSV)). Retroviruses” also include human T cell leukemia viruses, HTLV-1 and HTLV-2, and the lentiviral family of retroviruses, such as Human Immunodeficiency Viruses, HIV-1, HIV-2, simian immunodeficiency virus (SIV), feline immunodeficiency virus (FIV), equine immunodeficiency virus (EIV), and other classes of retroviruses.

A lentiviral vector for use herein refers to a vector derived from a lentivirus, a group (or genus) of retroviruses that give rise to slowly developing disease. Viruses included within this group include HIV (human immunodeficiency virus; including HIV type 1, and HIV type 2); visna-maedi; a caprine arthritis-encephalitis virus; equine infectious anemia virus; feline immunodeficiency virus (FIV); bovine immune deficiency virus (BIV); and simian immunodeficiency virus (SIV). Preparation of the recombinant lentivirus can be achieved using the methods according to Dull et al. and Zufferey et al. (Dull et al., J. Virol., 1998; 72: 8463-8471 and Zufferey et al., J. Virol. 1998; 72:9873-9880).

Retroviral vectors (i.e., both lentiviral and non-lentiviral) for use can be formed using standard cloning techniques by combining the desired DNA sequences in the order and orientation described herein (Current Protocols in Molecular Biology, Ausubel, F. M. et al. (eds.) Greene Publishing Associates, (1989), Sections 9.10-9.14 and other standard laboratory manuals; Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol 150:4104-4115; U.S. Pat. Nos. 4,868,116; 4,980,286; PCT Application WO 89/07136; PCT Application WO 89/02468; PCT Application WO 89/05345; and PCT Application WO 92/07573).

Suitable sources for obtaining retroviral (i.e., both lentiviral and non-lentiviral) sequences for use in forming the vectors include, for example, genomic RNA and cDNAs available from commercially available sources, including the Type Culture Collection (ATCC), Rockville, Md. The sequences also can be synthesized chemically.

For expression of a CAR, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art, as described above. For example, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vectors provided herein. The host cells containing the vector may be useful in expression or cloning of the polynucleotide contained in the vector. Suitable host cells can include, without limitation, prokaryotic cells, fungal cells, yeast cells, or higher eukaryotic cells such as mammalian cells. Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The CARs are introduced into a host cell using transfection and/or transduction techniques known in the art. As used herein, the terms, “transfection,” and, “transduction,” refer to the processes by which an exogenous nucleic acid sequence is introduced into a host cell. The nucleic acid may be integrated into the host cell DNA or may be maintained extrachromosomally. The nucleic acid may be maintained transiently or may be a stable introduction. Transfection may be accomplished by a variety of means known in the art including but not limited to calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Transduction refers to the delivery of a gene(s) using a viral or retroviral vector by means of viral infection rather than by transfection. In certain embodiments, retroviral vectors are transduced by packaging the vectors into virions prior to contact with a cell. For example, a nucleic acid encoding a CAR carried by a retroviral vector can be transduced into a cell through infection and pro virus integration.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well known in the art. See, for example, Sambrook et al. (2001, Molecular Cloning: A Laboratory Manual, Cold Spring Harbor Laboratory, New York).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

### Immune Effector Cells

In certain aspects, also disclosed herein are immune effector cells that are engineered to express the disclosed CAR polypeptides. In some embodiments, the cells are obtained from the subject to be treated (i.e., are autologous). However, in certain embodiments, immune effector cell lines or donor effector cells (allogeneic) are used.

Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. In preferred embodiments, the immune effector cells are obtained from TDLN.

A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

The present disclosure provides methods for making the immune effector cells which express the CARs described herein. In some embodiment, the method comprises transfecting or transducing immune effector cells isolated from a TDLN of a subject, such that the immune effector cells express one or more CAR as described herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR. In this regard, the immune effector cells may be cultured before or after being genetically modified (i.e., transduced or transfected to express a CAR as described herein).

Prior to in vitro manipulation or genetic modification of the immune effector cells described herein, the source of cells may be obtained from a subject. In some embodiments, the immune effector cells for use with the CARs as described herein comprise T cells. T cells can be obtained from a number of sources, including peripheral blood mononuclear cells, bone marrow, lymph nodes tissue, cord blood, thymus issue, tissue from a site of infection, ascites, pleural effusion, spleen tissue, and tumors. In preferred embodiments, T cells are obtained from a TDLN of a subject.

In one embodiment, the obtained cells are washed with PBS. In an alternative embodiment, the washed solution lacks calcium, and may lack magnesium or may lack many, if not all, divalent cations. As would be appreciated by those of ordinary skill in the art, a washing step may be accomplished by methods known to those in the art, such as by using a semiautomated flowthrough centrifuge. After washing, the cells may be resuspended in a variety of biocompatible buffers or other saline solution with or without buffer. In certain embodiments, T cells may be directly resuspended culture media.

A specific subpopulation of T cells, such as CD28+, CD4+, CD8+, CD45RA+, and CD45RO− T cells, can be further isolated by positive or negative selection techniques. For example, enrichment of a T cell population by negative selection can be accomplished with a combination of antibodies directed to surface markers unique to the negatively selected cells. One method for use herein is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD1 b, CD16, HLA-DR, and CD8. Flow cytometry and cell sorting may also be used to isolate cell populations of interest.

T lymphocytes may be further isolated and in certain embodiments, both cytotoxic and helper T lymphocytes can be sorted into naive, memory, and effector T cell subpopulations either before or after genetic modification and/or expansion. CD8+ cells can be obtained by using standard methods. In some embodiments, CD8+ cells are further sorted into naive, central memory, and effector cells by identifying cell surface antigens that are associated with each of those types of CD8+ cells. In embodiments, memory T cells are present in both CD62L+ and CD62L-subsets of CD8+ peripheral blood lymphocytes. T cells are sorted into CD62L-CD8+ and CD62L+CD8+ fractions after staining with anti-CD8 and anti-CD62L antibodies. In some embodiments, the expression of phenotypic markers of central memory TCM include CD45RO, CD62L, CCR7, CD28, CD3, and CD127 and are negative for granzyme B. In some embodiments, central memory T cells are CD45RO+, CD62L+, CD8+ T cells. In some embodiments, effector T cells are negative for CD62L, CCR7, CD28, and CD127, and positive for granzyme B and perforin. In some embodiments, naive CD8+T lymphocytes are characterized by the expression of phenotypic markers of naive T cells including CD62L, CCR7, CD28, CD3, CD 127, and CD45RA.

In certain embodiments, CD4+ T cells are further sorted into subpopulations. For example, CD4+T helper cells can be sorted into naive, central memory, and effector cells by identifying cell populations that have cell surface antigens. CD4+ lymphocytes can be obtained by standard methods. In some embodiments, naive CD4+T lymphocytes are CD45RO−, CD45RA+, CD62L+CD4+ T cell. In some embodiments, central memory CD4+ cells are CD62L positive and CD45RO positive. In some embodiments, effector CD4+ cells are CD62L and CD45RO negative.

The immune effector cells, such as T cells, can be genetically modified following isolation using known methods, or the immune effector cells can be activated and expanded (or differentiated in the case of progenitors) in vitro prior to being genetically modified. In another embodiment, the immune effector cells, such as T cells, are genetically modified with the chimeric antigen receptors (e.g., transduced with a viral vector comprising a nucleic acid encoding a CAR) and then are activated and expanded in vitro. Methods for activating and expanding T cells are known in the art and are described, for example, in U.S. Pat. Nos. 6,905,874; 6,867,041; 6,797,514; WO2012079000. Generally, such methods include contacting the isolated T cells with a stimulatory agent and costimulatory agent, such as anti-CD3 and anti-CD28 antibodies, generally attached to a bead or other surface, in a culture medium with appropriate cytokines, such as IL-2 (e.g., recombinant human IL-2). Anti-CD3 and anti-CD28 antibodies attached to the same bead serve as a “surrogate” antigen presenting cell (APC). In other embodiments, the T cells may be activated and stimulated to proliferate with feeder cells and appropriate antibodies and cytokines using methods such as those described in U.S. Pat. Nos. 6,040,177; 5,827,642; and WO2012129514.

In some embodiments, the immune effector cells comprise any leukocyte involved in defending the body against infectious disease and foreign materials. For example, the immune effector cells can comprise lymphocytes, monocytes, macrophages, dendritic cells, mast cells, neutrophils, basophils, eosinophils, or any combinations thereof. For example, the immune effector cells can comprise T lymphocytes, preferably cytotoxic T lymphocytes (CTLs).

T helper cells (TH cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including TH1, TH2, TH3, TH17, TH9, or TFH, which secrete different cytokines to facilitate a different type of immune response.

Cytotoxic T cells (TC cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4+ Treg cells have been described naturally occurring Treg cells and adaptive Treg cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8+T lymphocytes.

Natural-killer (NK) cells are CD56+CD3− large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization, and can eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

### Binding Properties of the Chimeric Antigen Receptors

As used herein, the term “binding” in the context of the binding of a chimeric antigen receptor to, e.g., a predetermined antigen, such as a cell surface protein or fragment thereof (or to an antigen bound to a cell surface protein such as an HLA molecule). Binding typically refers to an interaction or association between a minimum of two entities or molecular structures, such as an antigen-binding domain:antigen interaction. For instance, binding affinity typically corresponds to a KD value of about 10⁻⁷ M or less, such as about 10⁻⁸ M or less, such as about 10⁻⁹ M or less when determined by, for instance, surface plasmon resonance (SPR) technology in a BIAcore 3000 instrument using the antigen as the ligand and the antibody or chimeric antigen receptor as the analyte (or antiligand). Cell-based binding strategies, such as fluorescent-activated cell sorting (FACS) binding assays, are also routinely used, and FACS data correlates well with other methods such as radioligand competition binding and SPR (Benedict, CA, J Immunol Methods. 1997, 201(2):223-31; Geuijen, C A, et al. J Immunol Methods. 2005, 302(1-2):68-77).

Accordingly, in some embodiments, a chimeric antigen receptor of the present disclosure binds to the predetermined antigen or cell surface molecule (receptor) having an affinity corresponding to a KD value that is at least ten-fold lower than its affinity for binding to a non-specific antigen (e.g., BSA, casein). As described herein, a chimeric antigen receptor of the present disclosure can bind to an HLA-presented antigen described herein. According to the present disclosure, in some embodiments, the affinity of a chimeric antigen receptor with a KD value that is equal to or less than ten-fold lower than a non-specific antigen may be considered non-detectable binding.

The term “KD” (M) refers to the dissociation equilibrium constant of a particular antigen-binding domain:antigen interaction. There is an inverse relationship between KD and binding affinity, therefore the smaller the KD value, the higher, i.e. stronger, the affinity. Thus, the terms “higher affinity” or “stronger affinity” relate to a higher ability to form an interaction and therefore a smaller KD value, and conversely the terms “lower affinity” or “weaker affinity” relate to a lower ability to form an interaction and therefore a larger KD value. In some circumstances, a higher binding affinity (or KD) of a particular molecule (e.g., a chimeric antigen receptor) to its interactive partner molecule (e.g. antigen X) compared to the binding affinity of the molecule (e.g., chimeric antigen receptor) to another interactive partner molecule (e.g. antigen Y) may be expressed as a binding ratio determined by dividing the larger KD value (lower, or weaker, affinity) by the smaller KD (higher, or stronger, affinity), for example expressed as 5-fold or 10-fold greater binding affinity, as the case may be

The term “kd” (sec-1 or 1/s) refers to the dissociation rate constant of a particular antigen-binding domain:antigen interaction, or the dissociation rate constant of a chimeric antigen receptor. Said value is also referred to as the koff value.

The term “ka” (M-1×sec-1 or 1/M) refers to the association rate constant of a particular antigen-binding domain:antigen interaction, or the association rate constant of a chimeric antigen receptor.

The term “KA” (M-1 or 1/M) refers to the association equilibrium constant of a particular antigen-binding domain:antigen interaction, or the association equilibrium constant of a chimeric antigen receptor. The association equilibrium constant is obtained by dividing the ka by the kd.

The term “EC50” or “EC50” refers to the half maximal effective concentration, which includes the concentration of a chimeric antigen receptor that induces a response halfway between the baseline and maximum after a specified exposure time. The EC50 essentially represents the concentration of a chimeric antigen receptor where 50% of its maximal effect is observed. In certain embodiments, the EC50 value equals the concentration of a chimeric antigen receptor of the present disclosure that gives half-maximal binding to cells expressing an antigen (e.g., a tumor-associated antigen), as determined by e.g. a FACS binding assay. Thus, reduced or weaker binding is observed with an increased EC50, or half maximal effective concentration value.
In one embodiment, decreased binding can be defined as an increased EC50 chimeric antigen receptor concentration that enables binding to the half-maximal amount of target cells.
The present disclosure provides chimeric antigen receptors with antigen-binding domains derived from antibodies that bind a human antigen with high affinity (e.g., nanomolar or sub-nanomolar KD values).

According to certain embodiments, the present disclosure provides chimeric antigen receptors with antigen-binding domains derived from corresponding antibodies that bind human antigen (e.g., at 25° C.) with a KD of less than about 5 nM as measured by surface plasmon resonance. In certain embodiments, the corresponding antibodies bind an antigenic protein with a KD of less than about 20 nM, less than about 10 nM, less than about 8 nM, less than about 7 nM, less than about 6 nM, less than about 5 nM, less than about 4 nM, less than about 3 nM, less than about 2 nM, less than about 1 nM, less than about 800 pM, less than about 700 pM, less than about 500 pM, less than about 400 pM, less than about 300 pM, less than about 200 pM, less than about 100 pM, less than about 50 pM, or less than about 25 pM as measured by surface plasmon resonance.

The present disclosure also provides chimeric antigen receptors with antigen-binding domains that bind the antigenic protein with a dissociative half-life (t½) of greater than about 10 minutes or greater than about 125 minutes as measured by surface plasmon resonance at 25° C. In certain embodiments, the corresponding antibodies bind the antigenic protein with a t½ of greater than about 3 minutes, greater than about 4 minutes, greater than about 10 minutes, greater than about 20 minutes, greater than about 30 minutes, greater than about 40 minutes, greater than about 50 minutes, greater than about 60 minutes, greater than about 70 minutes, greater than about 80 minutes, greater than about 90 minutes, greater than about 100 minutes, greater than about 110 minutes, or greater than about 120 minutes, as measured by surface plasmon resonance at 25° C.

### Therapeutic Methods

The tumor-draining lymph nodes (TDLNs) are the primary sites of the development of anti-tumor immunity. Lymphocytes obtained from tumor draining lymph nodes provide a unique opportunity to treat cancer patients because the lymphocytes are specific to various cancer antigens. Accordingly, in some aspects, provided herein are methods of treating cancer in a subject by administering to the subject a composition comprising lymphocytes from TDLNs. In some embodiments, the lymphocytes comprise T cells (e.g., CD8+ T cells). In some embodiments, the lymphocytes are T cells (e.g., CD8+ T cells).

Immune effector cells expressing the CARs disclosed herein elicit a therapeutically beneficial immune response against cancer cells. For example, an anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to a cancer antigen.

CAR-expressing immune effector cells prepared as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some aspects, provided herein are methods of treating cancer (e.g., a solid tumor) in a subject by administering to the subject a composition comprising cells expressing a CAR polypeptide disclosed herein. In some embodiments, the methods provided herein further comprise conjointly administering to the subject a composition comprising cell that express a second CAR polypeptide comprising a 4-1BB domain in the costimulatory region of the CAR polypeptide. In some embodiments, the second CAR comprises at least one intracytoplasmic signaling region comprising a cluster of differentiation 3 zeta (CD3ζ) domain. In some embodiments, the second CAR comprises an extracellular domain specific for a cancer antigen. The second CAR polypeptide may comprise a cluster of differentiation 8 alpha (CD8α) peptide in the hinge/transmembrane region. Without being bound by theory, the immune cells expressing the first CAR (i.e., a CAR polypeptide comprising a CD28 domain in the co-stimulatory domain of the CAR) provides an initial burst to facilitate rapid killing of cancer cells, while the administration of immune cell by the 4-1BB provides sustained cancer cell killing, albeit at a lower level of killing when compared to administration of immune cells expressing the first CAR. 4-1BB/CD3z CAR is associated with persistence of CAR T cells in patients, which will in turn provide sustained cancer cell killing.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective amount of cells in the composition is at least 2 cells (for example, at least 1 CD8+ central memory T cell and at least 1 CD4+ helper T cell subset) or is more typically greater than 10² cells, and up to 10⁶ up to and including 10⁸ or 10⁹ cells and can be more than 10¹⁰ cells. The number of cells will depend upon the ultimate use for which the composition is intended as will the type of cells included therein.

In some embodiments, each dose of CAR cells (e.g., CAR-T cells) comprises at least about, about, or no more than about 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, or 9×10\hat{ }14 CAR cells. It is readily recognized in the art that “10\hat{ }n” means 10 to the n-th power.

In some embodiments, each dose of CAR cells comprises at least about, about, or no more than 1×10\hat{ }7 CAR cells. In some embodiments, each dose of CAR cells comprises at least about, about, or no more than 1×10\hat{ }8 CAR cells. In some embodiments, each dose of CAR cells comprises at least about, about, or no more than 5×10\hat{ }8 CAR cells. In some embodiments, each dose of CAR cells comprises at least about, about, or no more than 1×10\hat{ }9 CAR cells. In some embodiments, each dose of CAR cells comprises at least about, about, or no more than 1×10\hat{ }10 CAR cells.

The cells may be autologous or heterologous to the patient undergoing therapy. The cells may be allogenic. If desired, the treatment may also include administration of mitogens (e.g., PHA) or lymphokines, cytokines, and/or chemokines (e.g., IFN-γ, IL-2, IL-12, TNF-α, IL-18, and TNF-β, GM-CSF, IL-4, IL-13, Flt3-L, RANTES, MIP1α, etc.) to enhance induction of the immune response.

The CAR expressing immune effector cell populations may be administered either alone, or as a pharmaceutical composition in combination with diluents and/or with other components such as IL-2 or other cytokines or cell populations. Pharmaceutical compositions disclosed herein may comprise a CAR-expressing immune effector cell population, such as T cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions disclosed herein may be formulated for intravenous administration.

The anti-tumor immune response induced in a subject by administering CAR expressing T cells described herein using the methods described herein, or other methods known in the art, may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions disclosed herein, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, N.Y., N.Y.

Thus, provided herein are methods of treating an individual diagnosed with or suspected of having, or at risk of developing a malignancy, comprising administering to the individual a therapeutically effective amount of the CAR-expressing immune effector cells as described herein.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

Provided herein are methods of conjointly administering to the subject a second CAR polypeptide comprising a 4-1BB domain in the costimulatory region of the CAR polypeptide. The second CAR polypeptide may further comprise at least one intracytoplasmic signaling region comprising a cluster of differentiation 3 zeta (CD3ζ) domain and/or an extracellular domain specific for a low density cancer antigen and/or a peptide in groove cancer antigen. The second CAR polypeptide may comprise a cluster of differentiation 8 alpha (CD8α) peptide in the hinge/transmembrane region.

In certain embodiments, the disclosed CAR-modified immune effector cells are administered to a patient in conjunction with (e.g., before, simultaneously or following) or conjointly with any number of relevant treatment modalities, including but not limited to additional cancer treatments. In some embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) or conjointly with bone marrow transplantation, T-cell ablative therapy using either chemotherapy agents such as, fludarabine, external-beam radiation therapy (XRT), cyclophosphamide, or antibodies such as OKT3 or CAMPATH. In other embodiments, the cell compositions are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells. In additional embodiments, expanded cells are administered before or following surgery to treat cancer or pre-cancerous lesions in the subject. In preferred embodiments, at least one checkpoint inhibitor is administered conjointly with CAR therapy to the subject.

### Administration Regimens

According to certain embodiments of the present disclosure, multiple doses of the engineered cells may be administered to a subject over a defined time course. The methods according to this aspect comprise sequentially administering to a subject multiple doses of the cells. As used herein, “sequentially administering” means that each dose is administered to the subject at a different point in time, e.g., on different days separated by a predetermined interval (e.g., hours, days, weeks or months). The present disclosure provides methods which comprise sequentially administering to the patient a single initial dose, followed by one or more secondary doses, and optionally followed by one or more tertiary doses.

The terms “initial dose,” “secondary doses,” and “tertiary doses,” refer to the temporal sequence of administration of the engineered cells of the present disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of engineered cells, but generally may differ from one another in terms of frequency of administration. In certain embodiments, however, the amount of engineered cells contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In some embodiments of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½2, 26, 26½, or more) weeks after the immediately preceding dose. The phrase “the immediately preceding dose,” as used herein, means, in a sequence of multiple administrations, the dose which is administered to a patient prior to the administration of the very next dose in the sequence with no intervening doses.

The methods according to this aspect of the present disclosure may comprise administering to a patient any number of secondary and/or tertiary doses. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Indications

In certain aspects, provided herein are methods of treating cancer using a CAR T cell provided herein. In some embodiments, cancers that may be treated by methods and compositions provided herein include, but are not limited to, cancer cells from the cervix, anus, vagina, vulva, penis, tongue base, larynx, tonsil, bladder, blood, bone, bone marrow, brain, breast, colon, esophagus, gastrointestine, gum, head, kidney, liver, lung, nasopharynx, neck, ovary, prostate, skin, non-melanoma skin cancer (NMSC), cutaneous squamous cell carcinoma (SCC), stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell lung cancer (SCLC); non-small cell lung cancer (NSCLC); papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; thyroid cancer, thyroid carcinoma, metastatic thyroid carcinoma, malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the disclosed CAR-T cells can be used in combination with any compound, moiety or group that has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®). Examples of further chemotherapeutic agents include Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegal1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the subject is also administered an additional immunotherapeutic agent. Immunotherapy refers to a treatment that uses a subject's immune system to treat cancer, e.g., cancer vaccines, cytokines, use of cancer-specific antibodies, T cell therapy, and dendritic cell therapy.

In some embodiments, the subject is also administered an immune modulatory protein. Examples of immune modulatory proteins include, but are not limited to, B lymphocyte chemoattractant (“BLC”), C-C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C-C motif) Ligand 2 (“MCP-1”), Macrophage colony-stimulating factor (“M-CSF”), Monokine induced by gamma interferon (“MIG”), Chemokine (C-C motif) ligand 2 (“MIP-1 alpha”), Chemokine (C-C motif) ligand 4 (“MIP-1 beta”), Macrophase inflammatory protein-1-delta (“MIP-1 delta”), Platelet-derived growth factor subunit B (“PDGF-BB”), Chemokine (C-C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C-C motif) ligand 27 (“CTACK”), Chemokine (C-X-C motif) ligand 16 (“CXCL16”), C-X-C motif chemokine 5 (“ENA-78”), Chemokine (C-C motif) ligand 26 (“Eotaxin-3”), Granulocyte chemotactic protein 2 (“GCP-2”), GRO, Chemokine (C-C motif) ligand 14 (“HCC-1”), Chemokine (C-C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C-X-C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C-C motif) ligand 20 (“MIP-3 alpha”), C-C motif chemokine 19 (“MIP-3 beta”), Chemokine (C-C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C-C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRGl-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Rα, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C-X-C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-IACE-2, Albumin, AMICA, Angiopoietin 4, B-cell activating factor (“BAFF”), Carbohydrate antigen 19-9 (“CA19-9”), CD 163, Clusterin, CRT AM, Chemokine (C-X-C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNTI-inducible-signaling pathway protein 1 (“WISP-1”), and Receptor Activator of Nuclear Factor×B (“RANK”).

The disclosed CARs and immune cells expressing CARs can be used in combination with an immune checkpoint inhibitor. Immune Checkpoint inhibition broadly refers to inhibiting the checkpoints that cancer cells can produce to prevent or downregulate an immune response. Two known immune checkpoint pathways involve signaling through the cytotoxic T-lymphocyte antigen-4 (CTLA-4) and programmed-death 1 (PD-1) receptors. These proteins are members of the CD28-B7 family of co-signaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T cell proliferation. Checkpoint inhibitors include, but are not limited to aptamers and antibodies that block PD-1 (Nivolumab (BMS-936558 or MDX1106), CT-011, MK-3475, AMP-514), PD-L1 (MDX-1105 (BMS-936559), MPDL3280A, MSB0010718C), PD-L2 (rHIgM12B7, AMP-224), CTLA-4 (Ipilimumab (MDX-010), Tremelimumab (CP-675,206)), IDO, B7-H3 (MGA271), B7-H4, TIM3, LAG-3 (BMS-986016). The immune checkpoint inhibitor may be cemiplimab (REGN2810), nivolumab (BMS-936558, MDX-1106, ONO-4538), pembrolizumab (MK-3475, SCH 900475), atezolizumab (MPDL3280A, RG7446, RO5541267), durvalumab (MEDI4736, MEDI-4736), avelumab (MSB0010718C), ipilimumab (BMS-734016, IBI310, MDX-010), SHR1210, sintilimab (IBI308), spartalizumab (PDR001), tislelizumab (BGB-A317), pidilizumab, BCD-100, toripalimab (JS001), BAY 1905254, ASP 8374, PF-06801591, AMP-224, AB122, AK105, AMG 404, BCD-100, BI 754091, F520, HLX10, HX008, JTX-4014, LZM009, MEDIO680, MGA012, Sym021, TSR-042, PSB205, MGD019, MGD013, AK104, XmAb20717, RO7121661, CX-188, INCB086550, FS118, BCD-135, BGB-A333, CBT-502, CK-301, CS1001, FAZ053, HLX20, KN035, MDX-1105, MSB2311, SHR-1316, TG-1501, ZKAB001, INBRX-105, MCLA-145, KN046, M7824, LY3415244, INCB086550, CA-170, CX-072, ADU-1604, AGEN1181, AGEN1884, MK-1308, REGN4659, XmAb22841, ATOR-1015, PSB205, MGD019, AK104, XmAb20717, BMS-986249, tremelimumab, BMS-986258, BGB-A425, INCAGN02390, Sym023, JNJ 61610588, BI 754111, LAG525, MK-4280, REGN3767, Sym022, TSR-033, relatlimab, JTX-2011, MGD009, BMS-986207, OMP-313M32, MK-7684 or TSR-022.

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PD-L1 inhibitor comprises an antibody that specifically binds PDL1, such as BMS-936559 (Bristol-Myers Squibb) or MPDL3280A (Roche). In some embodiments, the PD-1 inhibitor comprises an antibody that specifically binds PD-1, such as lambrolizumab (Merck), nivolumab (Bristol-Myers Squibb), or MEDI4736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.
In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.
In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbB1 (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM 1 or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec STI571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with CARs (e.g., the CARs disclosed herein and immune cells expressing such CARs) for treating the disorders as described above may be an anti-cancer cytokine, chemokine, or combination thereof. Examples of suitable cytokines and growth factors include IFNy, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C-C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or ““regulating agent””). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRATL)/apoptosis-2 ligand (Apo-2L), antibodies that activate TRAIL receptors, IFNs, and anti-sense Bcl-2.

In some embodiments, a therapeutic agent for use in combination with CARs (e.g., the CARs disclosed herein and immune cells expressing such CARs) for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxyprogesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with CARs for treating the disorders as described above may be KD033. KD033 is a fusion antibody combining a fully human, high affinity anti-human Programmed Death Ligand 1 (PD-L1) IgG1 antibody with the human IL-15 receptor alpha (IL15Rα) sushi domain and human IL-15 (IL-15). KD033 (or its mouse cross reactive surrogate molecule, srKD033) has been extensively characterized in multiple invitro and in vivo nonclinical studies. The fusion of anti-PD-L1 antibody to IL-15 significantly increases the maximal-tolerated dose (MTD) of srKD033 in mice compared to free IL-15. In addition, srKD033 has exhibited increased efficacy in eliminating tumors in mice as compared to the combination of its individual components, anti-PD-L1 antibody and IL-15.

Combined administration, as described above, may be simultaneous, separate, or sequential. For simultaneous administration the agents may be administered as one composition or as separate compositions, as appropriate.

### Pharmaceutical Composition

Pharmaceutical compositions of agents suitable for injectable use (for administration of e.g., CAR cells, CAR T cells, and/or any additional cancer therapeutic agent) include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the disclosure in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by, and directly dependent on, (a) the unique characteristics of the active compound and the particular therapeutic effect to be achieved, and (b) the limitations inherent in the art of compounding such an active compound for the treatment of sensitivity in individuals.

The pharmaceutical composition can comprise any pharmaceutically acceptable ingredients, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

In various aspects, the pharmaceutical composition comprises formulation materials that are nontoxic to recipients at the dosages and concentrations employed. In specific embodiments, pharmaceutical compositions comprising an active agent and one or more pharmaceutically acceptable salts; polyols; surfactants; osmotic balancing agents; tonicity agents; anti-oxidants; antibiotics; antimycotics; bulking agents; lyoprotectants; anti-foaming agents; chelating agents; preservatives; colorants; analgesics; or additional pharmaceutical agents. In various aspects, the pharmaceutical composition comprises one or more polyols and/or one or more surfactants, optionally, in addition to one or more excipients, including but not limited to, pharmaceutically acceptable salts; osmotic balancing agents (tonicity agents); anti-oxidants; antibiotics; antimycotics; bulking agents; lyoprotectants; anti-foaming agents; chelating agents; preservatives; colorants; and analgesics.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. See, Remington's Pharmaceutical Sciences, 18” Edition, (A. R. Genrmo, ed.), 1990, Mack Publishing Company.

The pharmaceutical compositions can be formulated to achieve a physiologically compatible pH. In some embodiments, the pH of the pharmaceutical composition can be for example between about 4 or about 5 and about 8.0 or about 4.5 and about 7.5 or about 5.0 to about 7.5. In various embodiments, the pH of the pharmaceutical composition is between 5.5 and 7.5.

The present disclosure provides methods of producing a pharmaceutical composition. In various aspects, the method comprises combining the CAR cells (e.g., CAR T cells) and/or additional cancer therapy, with a pharmaceutically acceptable carrier, diluent, and/or excipient.

### Clinical Efficacy/Response to a Therapy for Cancer

Clinical efficacy can be measured by any method known in the art. For example, the response to a therapy relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al., J. Clin. Oncol. (2007) 25:4414-4422) or Miller-Payne score (Ogston et al., (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular anti-immune checkpoint therapeutic regimen is at least 25%, 30%, 35%, 40%, 45%, 50%, 55%, 60%, 65%, 70%, 75%, 80%, 85%, or more.

Additional criteria for evaluating the response to a cancer therapy are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include probability of survival, probability of metastasis within a given time period, and probability of tumor recurrence.

For example, in order to determine appropriate threshold values, a particular anti-cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following the cancer therapy for whom biomarker measurement values are known. In certain embodiments, the same doses of anti-cancer agents are administered to each subject. In related embodiments, the doses administered are standard doses known in the art for anti-cancer agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months.

### EXEMPLARY EMBODIMENTS

1. A lymphocyte from a tumor-draining lymph node (TDLN) of a subject afflicted with a cancer, wherein the lymphocyte comprises a CAR polypeptide comprising:
a) at least one intracytoplasmic signaling region comprising a cluster of differentiation 3 zeta (CD3ζ) domain, and
b) an antigen binding domain specific for a cancer antigen.

2. The lymphocyte of 1, wherein the CAR polypeptide further comprises at least one costimulatory region,
optionally wherein the at least one costimulatory region comprises
a) a cluster of differentiation 28 (CD28) domain,
b) a 4-1BB domain, or
c) both a) and b).

3. The lymphocyte of 1 or 2, wherein the CAR polypeptide further comprises a cluster of differentiation 8 (CD8) hinge transmembrane domain.

4. The lymphocyte of any one of 1-3, wherein the cancer antigen is selected from ICAM-1, GD2, Mesothelin, HER2, EGFR, PSMA, MUC1, L1-CAM, CD276 (B7-H3), CD44v6, IL13Rα2, EpCAM, FAP, CD133, ROR1, CD24, B7-H4, NKG2D ligands, CD47, GPC3, Claudin 18.2, and EGFRvIII.

5. The lymphocyte of 4, wherein the cancer antigen is ICAM-1.

6. The lymphocyte of any one of 1-5, wherein the antigen-binding domain is a single chain fragment variable (svFv) that binds ICAM-1 or an I domain of the αL subunit of lymphocyte function-associated antigen-1 (LFA1).

7. The lymphocyte of any one of 1-6, wherein the TDLN is resected from a benign tumor.

8. The lymphocyte of any one of 1-7, wherein the TDLN is resected from a subject at an early-stage cancer or an advanced-stage cancer.

9. The lymphocyte of any one of 1-8, wherein the TDLN is resected from a subject treated prior to or concurrently with at least one checkpoint inhibitor.

10. The lymphocyte of 9, wherein the at least one checkpoint inhibitor is a PD-1 inhibitor (e.g., an anti-PD-1 antibody), a PD-L1 inhibitor (e.g., an anti-PD-L1 antibody), KD033, or any combination of two or more thereof.

11. The lymphocyte of 10, wherein the PD-1 inhibitor is selected from MP-514 (MEDI0680), nivolimumab, cemiplimab, pembrolizumab, dostarlimab, Vopratelimab (JTX-4014), Spartalizumab (PDR001), Camrelizumab (SHR1210), Sintilimab (IBI308), Tislelizumab (BGB-A317), Toripalimab (JS 001), INCMGA00012 (MGA012), and AMP-224.

12. The lymphocyte of 10, wherein the PD-L1 inhibitor is selected from atezolizumab, durvalumab, avelumab, KN035, Cosibelimab (CK-301), AUNP12, CA-170, and BMS-986189.

13. The lymphocyte of any one of 1-12, wherein the lymphocyte is a T lymphocyte, a cytotoxic T lymphocyte (CTL), a regulatory T cell, an αβT cell, γδT cell, or any combination thereof.

14. The lymphocyte of of any one of 1-13, wherein the lymphocyte is a T lymphocyte.

15. The lymphocyte of any one of 1-14, wherein the lymphocyte expresses CD8 or CD4.

16. The lymphocyte of any one of 1-15, wherein the lymphocyte expresses PD-1 and/or CXCR5.

17. The lymphocyte of any one of 1-16, wherein the lymphocyte expresses CD8, PD-1, and CXCR5.

18. The lymphocyte of any one of 1-17, wherein the lymphocyte expresses at least one cell surface protein selected from CD62L, CD45RA, CCR7, CD28, IL-7Rα, CXCR3, and CD95.

19. The lymphocyte of of any one of 1-18, wherein the lymphocyte does not express CD45RO.

20. The lymphocyte of any one of 1-19, wherein the cancer is a solid tumor.

21. The lymphocyte of any one of 1-20, wherein the cancer is selected from non-small cell lung cancer (NSCLC), gastric cancer, Melanoma, Breast cancer, Lung cancer, Esophageal cancer, Gastric cancer, Pancreatic cancer, Liver cancer, Colorectal cancer, Anal cancer, Cervical cancer, Ovarian cancer, Endometrial cancer, Prostate cancer, Bladder cancer, Kidney cancer, Testicular cancer, Penile cancer, Head and neck cancer (including oral, pharyngeal, and laryngeal cancers), Thyroid cancer, metastatic thyroid carcinoma, Soft tissue sarcomas, and Neuroendocrine tumors, optionally wherein the cancer is selected from NSCLC, thyroid cancer, and metastatic thyroid carcinoma.

22. A pharmaceutical composition comprising the lymphocyte of any one of 1-21.

23. The pharmaceutical composition of 22, wherein the pharmaceutical composition comprises at least about 1×10\hat{ }7 cells, optionally at least about 1×10\hat{ }8 cells

24. A method of treating a subject afflicted with a cancer, the method comprising administering to the subject (a) a lymphocyte from a tumor-draining lymph node, (b) the lymphocyte of any one of claims 1-21, (c) the pharmaceutical composition of claim 22 or 23, or (d) any combination of two or more selected from (a)-(c).

25. The method of 24, wherein the lymphocyte autologous or allogeneic to the subject.

26. The method of 24 or 25, wherein the subject is administered with a single dose of the lymphocyte or pharmaceutical composition.

27. The method of 24 or 25, wherein the subject is administered with at least two doses of the lymphocyte or pharmaceutical composition.

28. The method of any one of 24-27, wherein the subject is treated conjointly with at least one additional cancer therapy.

29. The method of 28, wherein the subject is administered with the at least one additional cancer therapy concomitant with, prior to, or following the administration of the lymphocyte or the pharmaceutical composition comprising the lymphocyte.

30. The method of 28 or 29, wherein the at least one additional cancer therapy is selected from immunotherapy, checkpoint inhibitors, cancer vaccines, chemotherapy, radiation therapy, and surgery, optionally a checkpoint inhibitors.

31. The method of any one of 24-30, wherein the at least one additional cancer therapy is a PD-1 inhibitor (e.g., an anti-PD-1 antibody), a PD-L1 inhibitor (e.g., an anti-PD-L1 antibody), KD033, or any combination of two or more thereof.

32. The method of 31, wherein the PD-1 inhibitor is selected from MP-514 (MEDI0680), nivolimumab, cemiplimab, pembrolizumab, dostarlimab, Vopratelimab (JTX-4014), Spartalizumab (PDR001), Camrelizumab (SHR1210), Sintilimab (IBI308), Tislelizumab (BGB-A317), Toripalimab (JS 001), INCMGA00012 (MGA012), and AMP-224.

33. The method of 31, wherein the PD-L1 inhibitor is selected from atezolizumab, durvalumab, avelumab, KN035, Cosibelimab (CK-301), AUNP12, CA-170, and BMS-986189.

34. The method of any one of 24-33, wherein the cancer is selected from non-small cell lung cancer (NSCLC), gastric cancer, Melanoma, Breast cancer, Lung cancer, Esophageal cancer, Gastric cancer, Pancreatic cancer, Liver cancer, Colorectal cancer, Anal cancer, Cervical cancer, Ovarian cancer, Endometrial cancer, Prostate cancer, Bladder cancer, Kidney cancer, Testicular cancer, Penile cancer, Head and neck cancer (including oral, pharyngeal, and laryngeal cancers), Thyroid cancer, metastatic thyroid carcinoma, Soft tissue sarcomas, and Neuroendocrine tumors, optionally wherein the cancer is selected from NSCLC, thyroid cancer, and metastatic thyroid carcinoma.

35. The method of any one of 1-34, wherein the subject is a mammal, optionally a mouse, a dog, a cat, or a human.

### EXEMPLIFICATION

### Example 1: Manufacturing CAR-T Cells

The following exemplary method has been used to manufacture CAR-T cells.

### Day 0 Sample Processing & T Cell Isolation/Activation

### Mechanical Dissociation

1. Take samples under a sterile hood, use a petri dish, 70-micron filter and a pestle, place the sample in the 70 micron filter on the petri dish & add 3-5 ml of media and use the pestle to mechanically dissociate the tissue in a single cell suspension. The gentleMACS™ Dissociator, which is a benchtop instrument for the semi-automated dissociation of tissues into single-cell suspensions or thorough homogenates, can also be used instead of a pestle.
2. Transfer the cell suspension in a 50 ml conical tube with a coupled 70-micron filter.
3. Spin 1500 rpm×5 minutes & discard the supernatant.
4. Add 1 ml of ACK lysis buffer for 1 minute and during this time pipette the sample up/down at least 15 times, when time is done add 9 ml of media to block the reaction.
5. Spin 1500 rpm×5 minutes & discard the supernatant.
NOTE 1: if the cell suspension still has a lot of debris (black visible particles) filter the sample for a second time (also always filter blood samples), this will decrease the chance of clogging the column.
NOTE 2: Alternatively, steps 3-5 can be optional. For example, the dissociated cells can go directly from mechanical dissociation and filter into an automated prodigy device for CAR T cell transduction. The manufacturing process for CAR-T cells are described, for example, by Vedvyas et al. (2019) Scientific Reports 9(1):10634. doi: 10.1038/s41598-019-46938-7. Erratum in: Sci Rep. 2020 Jul. 27; 10(1):12733, which is incorporated herein by reference.

### CD4 CD8 Isolation

6. Determine cell number.
7. Add 80 ul of MACS media to the sample & resuspend, then add 10 ul of cd4 and CD8 beads to the cell suspension (This number of beads will help you select up to 10e+07 cells, if the amount of cells is higher double the amount of beads/MACS media proportionally), mix and incubate at 4 degrees for 15 minutes.
8. When time is done wash with 9 ml of MACS media to the cells and Spin 1500 rpm×5 minutes.
9. While cells are spinning prepare magnets and set LS column for positive selection, set a 15 ml conical tube below and label (this will collect the negative CD4/CD8 cells), wash the column one time with 3 ml of MACS media.
10. When cells are done spinning discard the supernatant & resuspend the cells in 500 ul of MACS media, pipette and add the cell suspension to the column (make sure the reservoir is empty from the previous step).
11. Perform 3 washing steps using 3 ml of MACS media every time, start the next wash only when the column reservoir is empty.
12. When the washes are done remove the column and place on a new 15 ml conical tube, add 5 ml of MACs media and let the media flow through the column without flushing with the plunger, this collected portion is your CD4+CD8+ Cells.
NOTE: Avoid using the plunger at any point unless the column gets clogged, if the column gets clogged during the washes, avoid using the plunger at the end of the wash or you might lose part of you positive Cells.
13. Count T cells under the microscope & plate according to the conditions of your experiment, media should have a concentration of IL-7/IL-15 of 25 ng/ml each (1 ul of stock per ml of media), the concentration of beads must be calculated every time as the ratio.
Beads: Cells must be 1:1, keep in mind each mL of beads have 4×10e+07 beads to aliquot the volume of Dynabeads you need.
14. Dynabeads preparation for T cell activation:
   1) Vortex the vial of Dynabeads for 30 seconds
   2) Transfer the desired volume to a 15 ml conical tube.
   3) Add an equal volume of buffer or at least 1 ML and vortex.
   4) Either spin down or use a magnet on the side to get rid of the media and resuspend on a desirable volume of media to have a ratio Beads:Cells 1:1.
At the end of the process, you must have 2 million cells/ml of media (IL-5/IL-7 25 ng/ml beads: cell ratio 1:1, i.e., 2 million beads on 1 ml of media).
15. set cultures on an incubator at 37 C 5% CO2.

### Transduction D1

After 24 hours of activation with DYNABEADS
1. Plate cells to have 1 million cells/ml add virus to have a dilution of 1:12.

### Transduction D2:

1. Add virus again to have a dilution 1:12.

### D3-D10 Expansion

1. Check the cultures every day and add media+ cytokines at least every 3rd day or more frequently depending on the day, transfer the cells to a bigger plate if necessary.

### D10 Evaluation of Phenotype/Transduction Efficacy Freezing of Samples

Test for CMYC expression/memory phenotype, freeze samples using freezing-media: culture-media ratio of 2:1.

TABLE 2
Exemplary agents for preparing CAR T cells

| Items | Company | Catalog# |
|---|---|---|
| TexMACSTM GMP Medium | Miltenyi Biotec | 170-076-307 |
| Human Serum | Sigma-Aldrich | H4522 |
| Human IL-15 recombinant protein | ThermoFisher | PHC9151 |
| Human IL-7 recombinant protein | Gibco | PHC0076 |
| Dynabeads ™ Human T-Expander | ThermoFisher | 11141D |
| CD3/CD28 | | |
| CD8 MicroBeads, human | Miltenyi | 130-045-201 |
| CD4 MicroBeads, human | Miltenyi | 130-045-101 |
| CryoStor ® CS10 Freeze Media | BioLife Solutions | 210102 |
| MACS LS columns | | |
| Primocin | In vivogen | Ant-pm-2 |

### Initial Media Preparation for Cell Culture/Basal Stock (1L of Media):

to 1L texmacs media.
add 2 ml of Primocin 50 mg/ml to have a final concentration of 100 μg/ml.
add 50 ml of human serum to have a final concentration of 5%.
label, keep at 4° C.

### Media Preparation for Cell Culture Adding Cytokines:

Must be prepared always fresh, first calculate the mL's of media you need for culture & add IL-15/IL-7 from stock to have a final concentration of 25 ng/ml of each cytokine.
IL-15 1 ul of stock is 25 ng ul.
IL-7 1 ul of stock is 25 ng ul.
Beads: depending on the number of cells, 1 ml of Dynabeads have 4×10E+07 beads, aliquot the amount of volume you need to have a final ratio bead:cells 1:1 for activation. (Also follow beads preparation steps).

### MACS Media Preparation (1L of Media):

to 1L of PBS.
add 5 ml of BSA to have a final concentration of BSA 0.5%.
add 4 ml of EDTA 0.5 M to have a final concentration of 2 mM EDTA.
label, keep at 4° C.

### Example 2: Utilizing Lymph Node Derived CAR T Cells (LN-CARs)

The majority of surgical resections for solid tumors include a complete lymph nodal dissection of the surrounding lymph nodes for staging purposes. In early-stage lung cancers, all mediastinal and hilar lymph nodes are removed. The lymph nodes of cancer patients are utilized for harvesting the T cells for CAR T cell transduction as described in Example 1. Once the cells are expanded, they are adoptively transferred back into the patient. This strategy may be effectively used on patients receiving immune checkpoint inhibitors prior to resection because as demonstrated herein, treatment with checkpoint inhibitors expands these T cell populations in the TDLNs. In addition, the CAR T-cells can be stored under cryopreservation for future use.

### Example 3: Materials and Methods for Examples 4-6

We collected paired single cell (sc) RNA and TCR sequencing of patients. Blood, tumor, and tdLN sample were collected from each patient. We combined tdLN levels into one tissue sample for downstream analysis, giving us a total of 9 tissue samples. After filtering out non-CD8 T cells through FACS and sc sequencing techniques, we ended up with 40,974 T cells. We then used TCR sequencing as a clonal barcode to track tumor-specific clones across different tissues. If a clone found in the tumor was found in another tissue it was labeled as tumor matched (tm). Based on the literature, expanded clones were considered to have at least 10 T cells per clone. 50% of all T cells were a part of an expanded tm clone. The patient who did not receive treatment had a total of 136 clones expanded clones relating to the tumor, where approximately 42% were only found in the tumor (non-tm tumor clones).

To understand tm clonal phenotype we generated a UMAP of all samples and T cells, which was integrated based on patients. We generated 14 total clusters: cluster 1, 4, 13 represented a differentiated and effector-like phenotypes; clusters 5,6,7,9 had a cytotoxic effector and effector-memory-like phenotype; clusters 0,8,10, 11 exhibited a more memory phenotype; and lastly, clusters 2 and 3 had a quiescent, naive, and central memory-like phenotype. A tissue-specific clustering was found. The tdLN T cells were clustered in the most naive-like clusters, blood T cells ranged from these naive-like clusters to more cytotoxic memory clusters, whereas tumor T cells were found in the more differentiated effector-like clusters. Although tm T cells were found across many clusters, blood and tdLN expanded tm T cells were found in differentiated than other T cells, and fewer were found in very naive-like clusters. Larger clone sizes were also found in more differentiated clusters and tm clones had larger clone sizes than non-tm clones. When comparing all clone sizes, expanded and non-expanded, tm clones have a statistically significant higher clone size than non-tm clones. The largest 20 clone's tm clones for each patient are also larger than largest 20 non-tm clones.

We then explored phenotypic differences between expanded tm clones and patient tissue and response. Within the blood, patients had a higher cytotoxic and lower naive/cm score. The tdLN T cells followed a similar pattern. In addition, the patient. The tumor had very low Naïve/cm-like scoring.

### Methods:

After paired TCR and RNA single cell sequencing, tissue samples were aligned using 10× Genomics multi tool with the GRCh38 human reference. Samples were combined into one Seurat object and the amino acid CRD3 sequence from the TCR was added as meta data. Cells that did not include at least 1 TRA and TRB sequences were filtered out, as done in the literature. T cells were then filtered to remove non-CD8 T cells by first creating a UMAP of all T cells and then removing clusters with low average expression of CD8 Transcription factors and high expression of transcription factors of non-CD8 T cells (ex: MS4A1, EPCAM1, CD4). The UMAP was created using the steps suggested by Seurat and was integrated using RunHarmoney to reduce patient-specific sample biased. Next, a new UMAP was generated where each cluster had unique T cell features. This was determined by differential expression of all clusters using FindAllMarkers from Seurat and generating a DotPlot of transcription factors that describe CD8 T cell state. The UMAP was further analyzed to determine cluster function. Specifically, a transcriptional score of cytotoxic-like behavior (PRF1, IFNG, NKG7, GZMB, GZMA, GZMH, KLRK1, KLRB1, KLRD1, CTSW, CST7) was created using AddModuleScore. In addition, a second score was generated using AddModuleScore based on Pauken et al. description of naïve central memory T cells. We then overlayed these scores as well as transcriptional factors such as SELL and GZAMB onto the UMAP.

To understand the phenotype of tumor-relevant clones in other tissues, tumor-relevant clones had to be established. Similar to the approach outlined by Pauken et al and other, a clone was defined by having the same CDR3 amino acid sequence for all TRA and TRB sequences. If a clone was found in the tumor and in another tissue, we denoted it as tumor matching (tm). Tm clones were also further categorized by clones with matching TCRs found in the tumor and tdLN (tumor matched tdLN), by clones with TCRs found in the tumor and blood (tumor matched blood), and clones with TCRs found in the tumor, tdLN and blood (tumor matched tdLN & blood). Clone size was determined by the number of T cells within a given clone. Expanded clones were defined to have at least 10 T cells in the clones. To determine clonal diversity we found the total number of individual non-tm and tm clones (separated by category). We then compared meta data, such as transcriptional scores and clone size, within all expanded tm clones by patient and or tissue. We also compared meta data between non-tm and tm T cells within each patient.

Boxplots were visualized using ggplot2 package. Statistical tests were performed using the Tukey Honest Significant Differences post-hoc test using the rstatix package and ggpubr was used to visualize statistical significance. Other packages used for data manipulation, analysis, and visualization were stringr, RColorBrewer, readr, tidyverse, tibble, ggrepel. Excel was used to make stacked barcharts.

### Example 4: Tumor Relevant Stem Like T Cells are Located Primarily in the tdLN

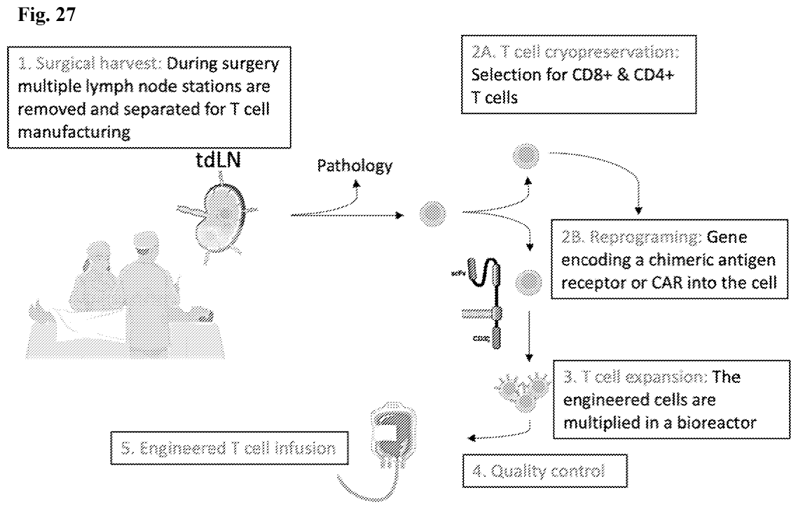

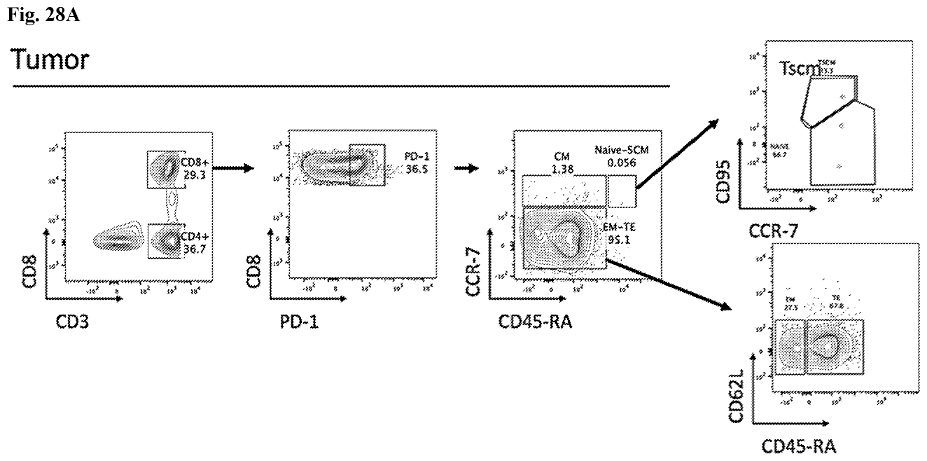

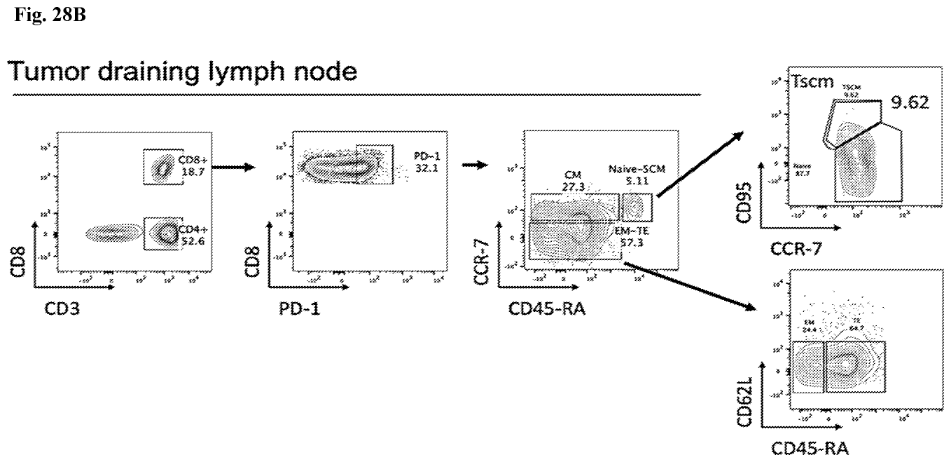

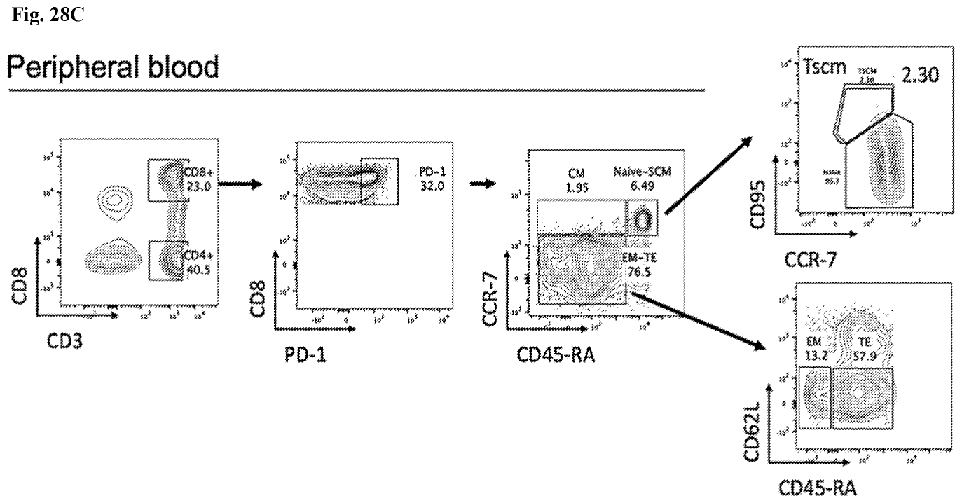

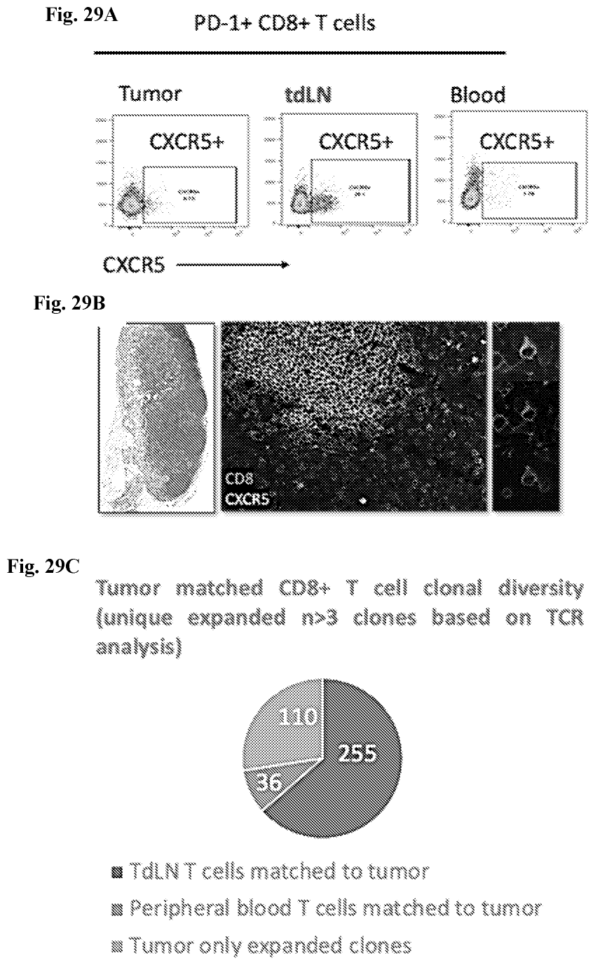

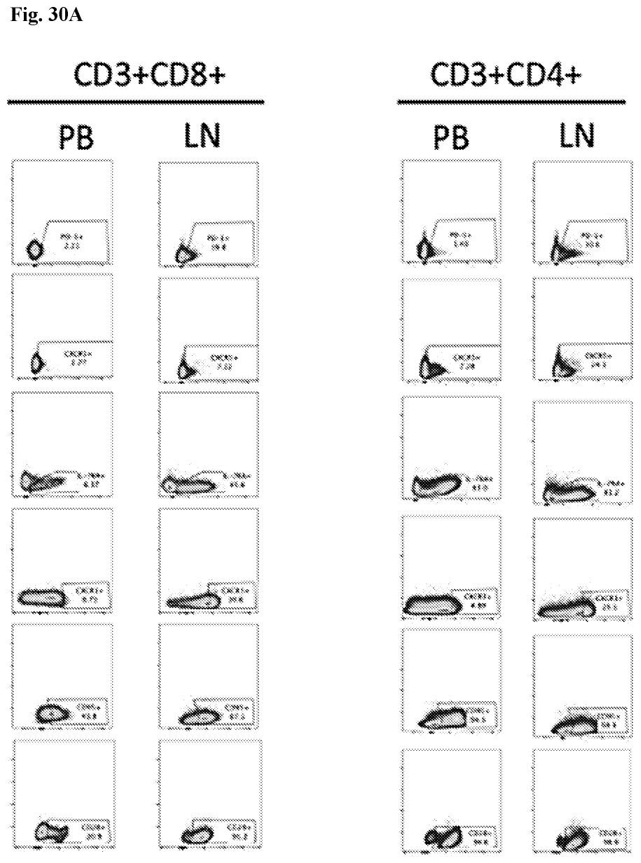

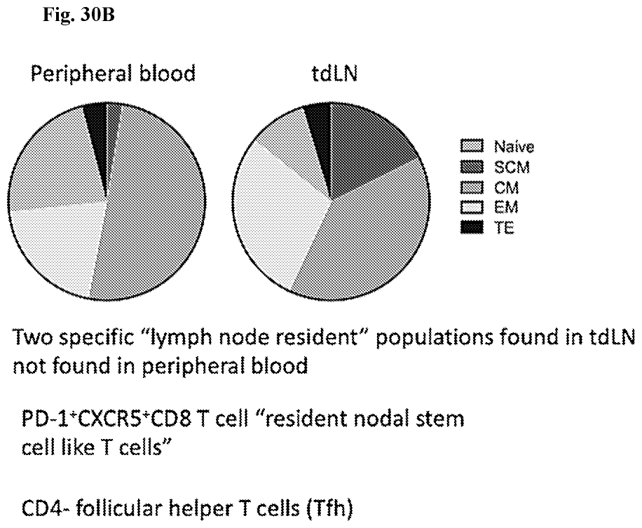

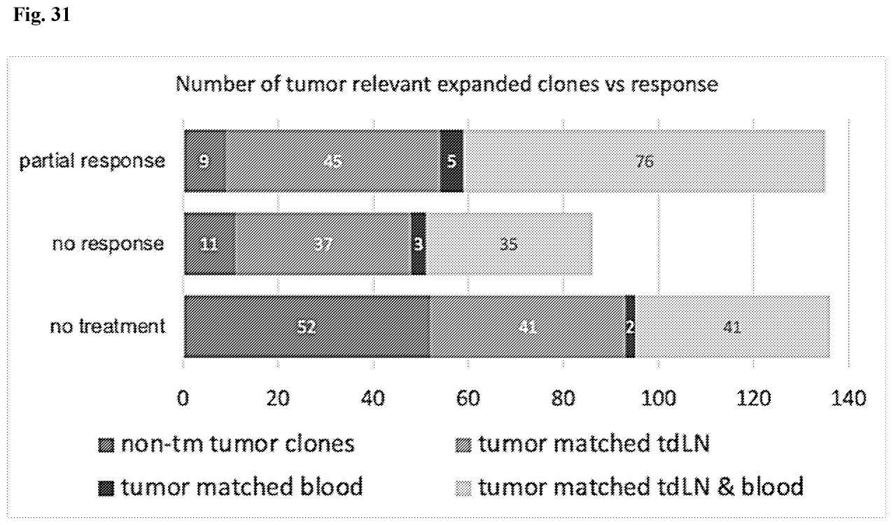

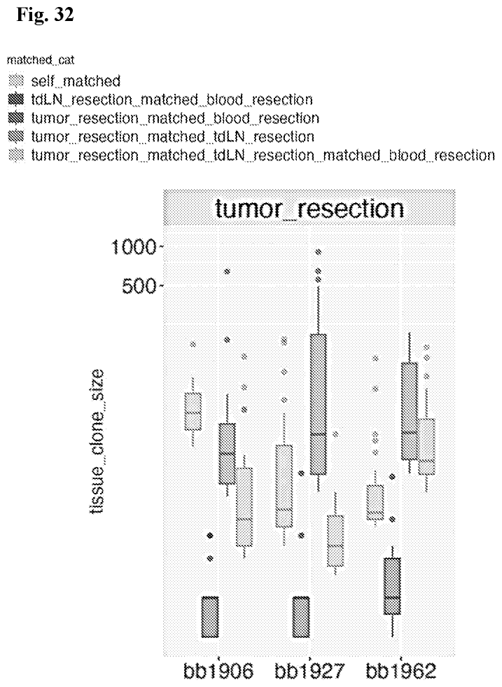

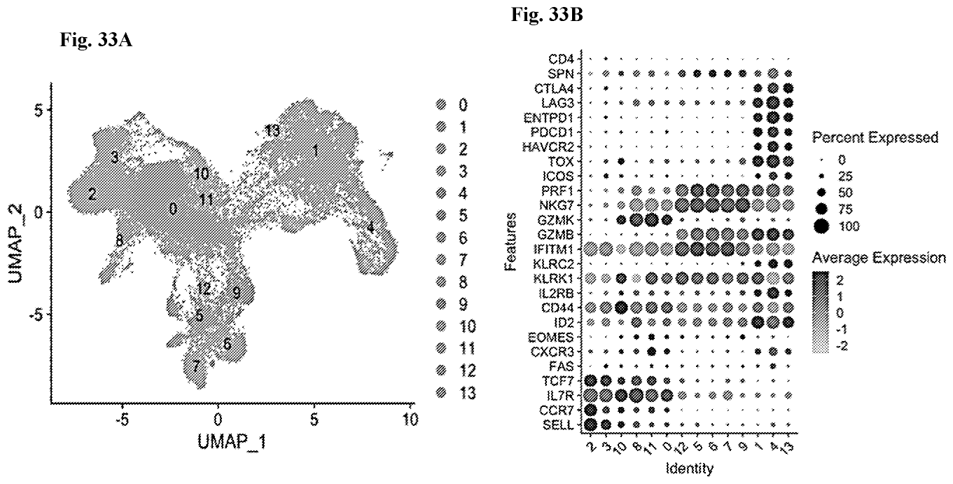

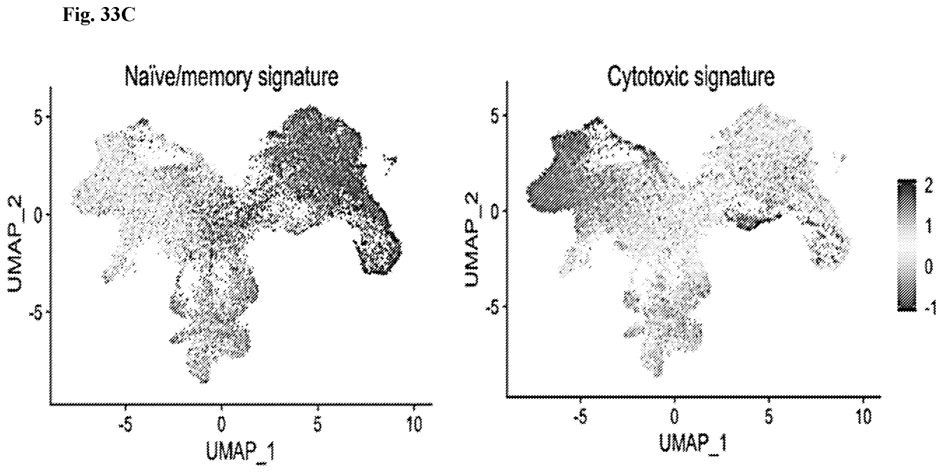

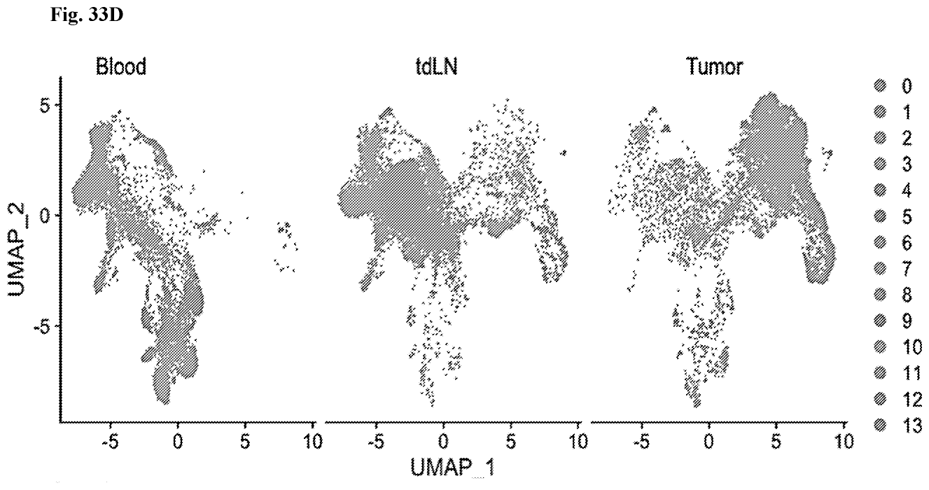

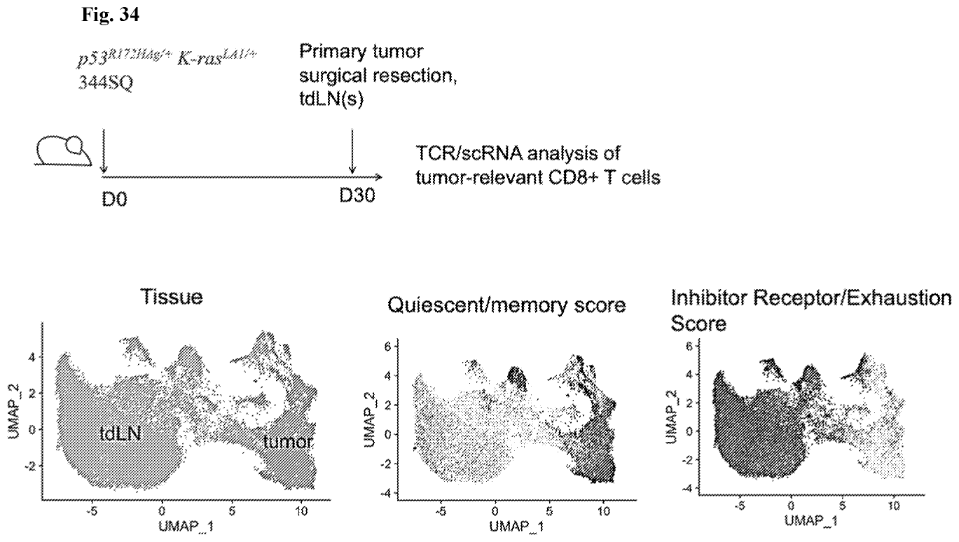

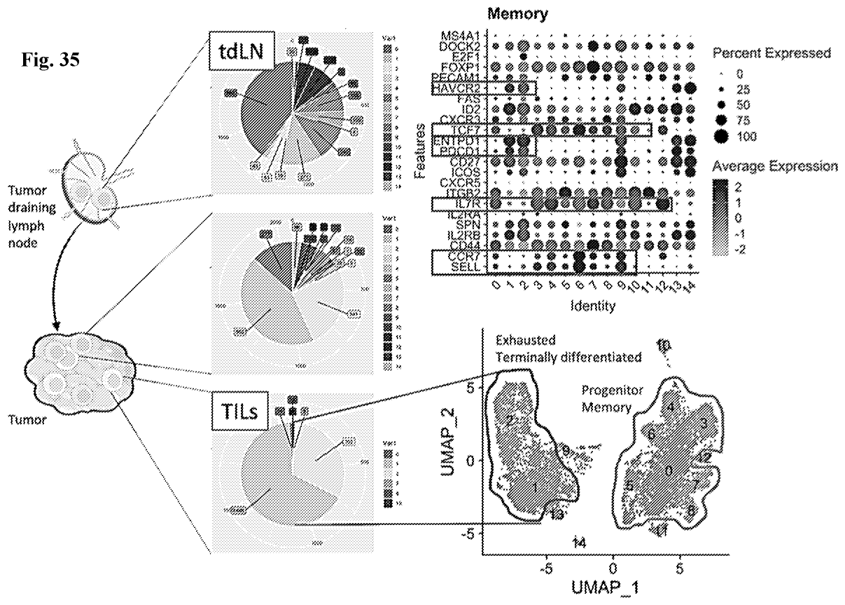

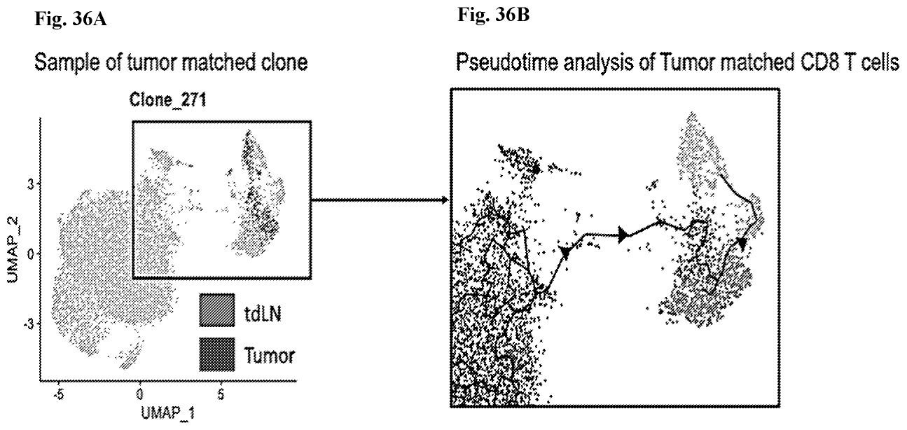

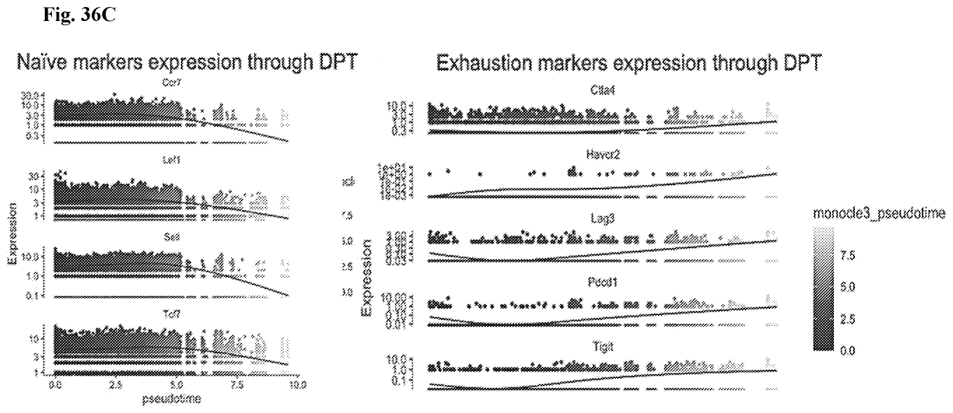

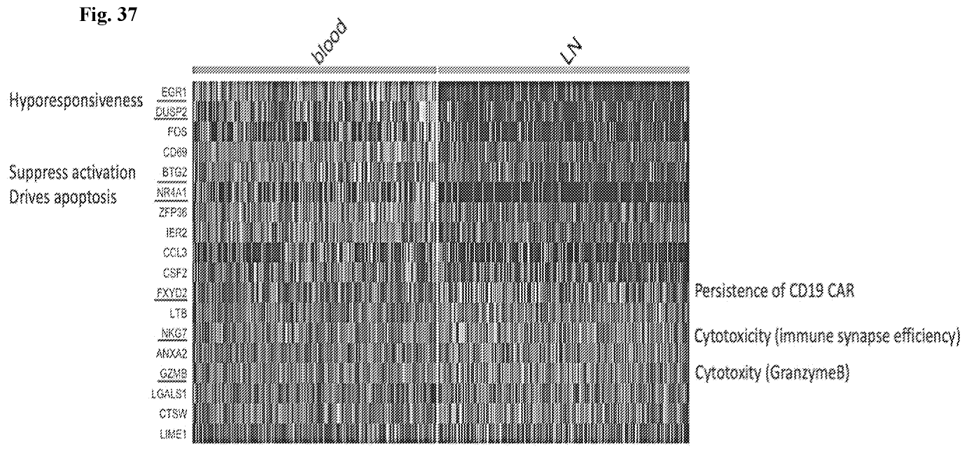

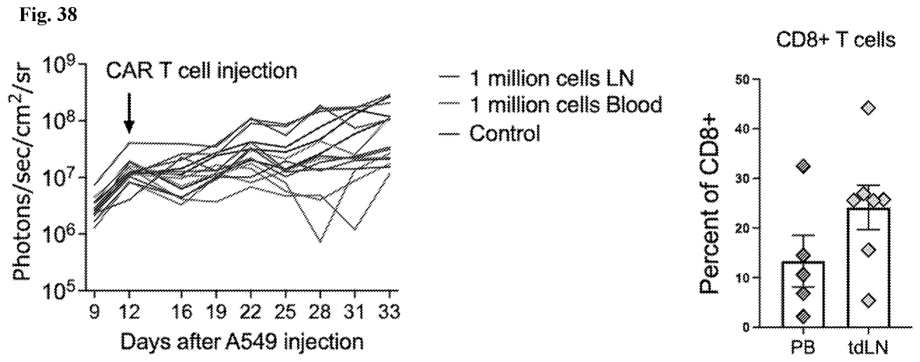

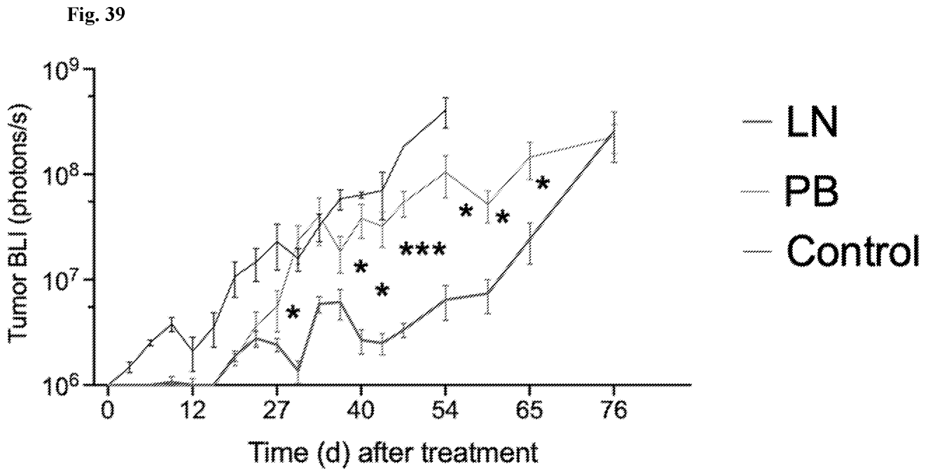

Our NSCLC tdLN analysis revealed T cell subsets with stem-cell memory characteristics, as indicated by PD-1+, TCF1hi, CXCR5+, and CD8+ expression, which were not significantly found in the tumor or in peripheral circulation both in a relevant mouse model (FIG. 27) and in patients (FIG. 28, FIG. 29, FIG. 30). These T cells exhibited progenitor-like transcriptional signatures, enhanced SELL and TCF7/TCF-1 expression, fewer exhaustion markers, and superior in vitro proliferation compared to TILs from both a murine lung cancer model and patient-derived tissues. Single cell (sc)RNA sequencing, coupled with TCR “tumor matching” (TM) techniques, exposed a rich clonal diversity of tumor-relevant clones within tdLNs, which showcased a broader transcriptional memory profile and distinct CD4+ and CD8+ phenotypes.

### Example 5: The tdLN as a Diverse Repository of T Cell Memory in NSCLC Patients

In our initial results, we discerned a “stem-cell memory-like” cluster (marked by PD-1+ TCF1hi CXCR5+CD8+ T-cells) found primarily in the tdLN of patients in the early stages of resectable lung cancer (FIG. 28A, FIG. 28B). This cluster mirrored the memory signatures of a PD-1 receptive progenitor memory CD8+ T cell that we defined using our murine model (specifically high stem cell-like, high SELL, TCF-1 hi, scarred memory signature, low exhaustion score, low cytotoxicity score, low cell stress score). To understand tumor-relevant T cell responses without a model antigen in both our murine platform and human subjects, we opted to utilize TCR sequencing and scRNA sequencing on CD8+ T cells sourced from varied tissues. Each chosen cell was confirmed to have annotations for at least one α and one β chain within the TCR data. This enabled us to categorize cells as tumor-matching (TM) or non-matching, contingent on the identical a and R chain composition present in the paired tissue data²⁵⁻²⁶. We recognize that both human and murine CD8+ tumor-infiltrating lymphocytes may target tumor antigens or unrelated epitopes, the latter indicated by a lack of CD39 expression. To ensure our analysis focused on tumor-specific T cells, we excluded bystander viral CD8+ T cells, which are typically non-tumor specific²⁷. Additionally, we eliminated TCR sequences linked to common pathogens like CMV and EBV, using databases such as VDJ.com, IEDB (Immune Epitope Database and Analysis Resource), MHCcluster, and EPINMC. This approach helped us avoid the influence of non-tumor related T cell responses. Using TCR/scRNA seq, we examined CD8+ T cells from matched tdLN, peripheral blood, and tumor tissue in NSCLC patients. In the tdLN compared to the peripheral blood of patients, there was significant clonal diversity in tumor-relevant clones (TM) (FIG. 28C). The transcriptional diversity of these clones was more pronounced than that observed in the non-tdLN derived TIL population. Our findings suggest that CD8+ T cells originating from tdLN undergo significant diversification, adopting varied functional states and eventual exhaustion upon infiltrating tumors (FIG. 29). Thus, sourcing tumor relevant clones directly from the tdLN might offer a selection enriched in less exhausted tumor-responsive memory subsets.

In our initial research on NSCLC, we harvested T cells from the tumor draining lymph nodes (tdLN) of NSCLC patients and assessed their in vivo efficacy in a murine model using the human A549 lung adenocarcinoma cell line (FIG. 30). We conducted serial bioluminescence imaging (BLI) with firefly luciferase (ffLuc)-transduced A549 to monitor tumor establishment and response to T cell therapy. Mice with established lung tumors (n=10 per group) received a single intravenous dose of 5×10\hat{ }6 LN-ICAM-1 CAR T cells 12 days post-tumor inoculation, showing substantial tumor control compared to controls.

The data presented herein demonstrate that CAR T cells engineered from tdLN significantly outperforms their leukapheresis derived conventional counterparts in proliferation, biodistribution, and persistence, thereby yielding a more potent anti-tumor response in solid tumors. Our results have underscored the efficacy of CAR T cell therapy using ICAM-1 targeted lymphocytes derived from tdLNs in a murine model of NSCLC. To substantiate these results, we utilized a cohort of mice for a head-to-head comparison of matched conventional peripheral blood and tdLN constructed CARs. We used bioluminescence imaging (BLI) and PET to evaluate tumor burden, track survival, as well as analyze T cell biodistribution and T cell persistence. To show the superiority of stem cells that recognize multiple tumor antigens, we employed TCR/scRNA sequencing, to track individual clonal expansion, clonal diversity, and transcriptional profiles. We show herein that LN-CAR is superior to conventional peripheral CAR T cells for the treatment of heterogenous solid tumors by several factors including persistence, endogenous tumor antigen recognizing T cells.

Further demonstrated herein are reproducible isolation, expansion, and transduction of CD4+ and CD8+ T cells from the tdLNs of several lung cancer patients (n=6). The methodology we have developed can efficiently generate a robust number (5×10⁸ to 1×10⁹) of LN-ICAM-1 CAR that are comparable to the goal dose of a current ICAM-1 targeting phase I clinical trial (NCT04420754) and ready for infusion in 2 weeks. We conducted detailed immunophenotyping of the adoptively transferred cells at 30 days post-transfer) timepoints in tumor-bearing lungs. Single cell sequencing (n=10,000 cells per sample) was also be performed to discern transcriptional changes and signatures.

### Adoptive Cell Therapy for Solid Tumors.

Lung cancer remains the leading cause of death for both men and women worldwide, with non-small cell lung cancer (NSCLC) accounting for 85% of cases¹. The therapeutic strategies for NSCLC have largely focused on modulating the PD-1/PD-L1 axis via immune checkpoint blockade (ICB). Despite remarkable clinical results with a potential for cure, response rates have plateaued at approximately 13-20%, and novel combination partners to enhance this response have been incremental, leaving the majority of patients with few treatment options and a poor prognosis²⁻⁴. This suboptimal response is often attributed to the tumors' immunologically ‘cold’ phenotype, characterized by a scarcity of tumor-infiltrating lymphocytes (TILs)⁵. A promising strategy involves augmenting the tumoricidal lymphocyte population through the infusion of autologous TILs, which are harvested from patient-derived tumor tissues and expanded ex vivo. This adoptive transfer approach has proven effective across various solid tumor types, thanks in part to the polyclonal nature of the T cell populations, which are adept at recognizing multiple tumor antigens, as well as the potential lifespan of memory cells that can last for decades⁶⁻⁹. However, the process of extracting and isolating TILs is complex, and the lengthy ex vivo expansion (6-8 weeks) often results in irreversible T cell exhaustion, further compromising their cytotoxic capability and persistence after infusion.

### Tumor Draining Lymph Node as a Source for Adoptive Cell Therapy.

In TIL therapy, the presence of mutation-specific T cells is essential, yet the differentiation lineage of these T cells is of greater significance. In melanoma TIL treatments, CD8+ T cells with stem cell-like phenotypic markers demonstrated a strong association with effective tumor lysis and durable clinical outcomes¹⁰. Notably, the bulk of tumor neoantigen-specific T cells were in a terminally differentiated-exhausted state, lacking a positive clinical correlation. This underscores that both antigenic specificity and T cell differentiation lineage are key determinants in the success of TIL therapy. Our research has shown that the tumor-draining lymph node (tdLN) is a niche residency for PD-1+TCF-1+ CXCR5+CD8+ T cells that exhibit stem cell-like qualities and tumor antigen-specific specificity, and play an indispensable role in persistent immunity. These stem cell memory (SCM) CD8+ T cells are similar to those found in other models of cancer and chronic virus in that they retain a level of differentiation plasticity akin to bona fide stemness, enabling them to embark on varied differentiation pathways orchestrating systemic immunity¹¹⁻¹⁶. In contrast to TILs, these T cells in tdLN have not only the capacity to recognize a multitude of tumor antigens but are transcriptionally and epigenetically divergent from the exhausted T cells typically found within tumors. TdLN-SCM cells are free from the epigenetic alterations that mark other T cell subsets, specifically in gene regions crucial to the regulation of T cell exhaustion¹¹. This lack of epigenetic “scarring” permits TdLN-SCM cells to evolve into fully functional effector T cells, comparable to canonical memory T cells. Moreover, the lymph nodes' microenvironment, which supports the maintenance of stem-like CD8+ T cells, likely serves as a niche for particular CD4+ T cell subpopulations that may enhance the function of stem-like CD8+ T cells. These CD4+ T cell subsets, such as CXCR5+CD4+ follicular helper (fh) and CXCR3+ T cell subsets, secrete memory-inducing cytokines such as IL-21 and are rarely present in peripheral blood or tumors¹⁷. CD4+ T cells, frequently incorporated in ACT formulations, can mediate direct antitumor responses and are critical for the sustained survival of CD8+ T cells within the context of ACT¹⁸. Thus, the integration of these lymphoid resident CD4+ T cell subsets with SCM CD8+ T cells has the potential to considerably enhance the therapeutic impact of ACT.

### a CAR T Cell Strategy.

The adaptability of tumor cells, through mechanisms like MHC-I molecule downregulation, poses a significant challenge to the effectiveness of endogenous T cells that target tumor cognate antigens¹⁹. To counter this, we are enhancing lymph node-derived T cells with a synthetic CAR T cell receptor that targets ICAM-1, a surface glycoprotein we and others have shown to be overexpressed in NSCLC tumors²⁰⁻²¹. We are utilizing a third-generation CAR that targets ICAM-1 and is currently undergoing a Phase I trial (NCT04420754)²²⁻²⁵. This trial has demonstrated initial safety and efficacy signals in treating metastatic thyroid carcinoma. This CAR design is novel, utilizing a precision-engineered LFA-1 receptor in place of the traditional monoclonal antibody scFv component, which increases specificity for ICAM-1-expressing cancer cells while maintaining safety for tissues expressing normal levels of ICAM-1.

### Conclusion

Our data pioneered the utilization of stem-like T cells derived from tdLN as a potent new strategy for genetically engineered ACT targeting solid tumors.

### Example 6: T Cells Selected from Lymph Node Acquisition for Adoptive Cell Therapy of NSCLC

Background: The primary limitation of PD-1 inhibitors in non-small cell lung cancer (NSCLC) arises from their inability to act on ‘cold’ tumors without tumor-reactive T cells, necessitating alternative approaches. Adoptive cell therapy (ACT), utilizing either autologous tumor-infiltrating lymphocytes (TILs) or chimeric antigen receptor (CAR)-engineered cells, strives to enhance antitumor immunity but faces several challenges such as identifying safe antigens, managing tumor heterogeneity that results in antigen escape, improving cell trafficking, and maintaining T cell persistence. To address these issues, we explored a new source of T cells from the benign tumor draining lymph nodes (tdLNs) of NSCLC patients. Our initial results have shown that tdLNs serve as a reservoir for a diverse and polyclonal set of tumor-relevant ‘stem-like’ T cells. We posit that employing these pluripotent T cells for ACT could achieve significant tumor rejection in NSCLC. Methods: Resected tumors, tdLN, non-draining (ndLN), and PB from NSCLC patients, as well as a syngeneic murine lung cancer model (344SQ), underwent analysis. T cells were profiled for using flow cytometry, complemented by cytokine and proliferation assays. TCR and single cell (sc)RNA sequencing were utilized to assess clonal expansion, diversity, and transcriptional profiles of tumor-relevant T cells. T cells were then transduced with an ICAM-1 targeting CAR, and in vivo efficacy was evaluated in an A549 murine lung cancer model.

Results: Our NSCLC tdLN analysis revealed T cell subsets with stem-cell memory characteristics, as indicated by PD-1+, TCF1hi, CXCR5+, and CD8+ expression, which were not significantly found in the tumor or PB. These T cells exhibited progenitor-like transcriptional signatures, enhanced SELL and TCF-1 expression, fewer exhaustion markers, and superior in vitro proliferation compared to TILs from both a murine lung cancer model and patient-derived tissues. scRNA sequencing, coupled with TCR “tumor matching” (TM) techniques, exposed a rich clonal diversity of tumor-relevant clones within tdLNs, which showcased a broader transcriptional memory profile and distinct CD4+ and CD8+ phenotypes. Upon analyzing the top 100 expanded (n>3) TM clones, 47 featured the presence of tdLN-derived T cells, covering progenitor, stem cell-like, and central memory clusters. T cell subsets were then transduced with a CAR targeting ICAM-1—a cell surface protein frequently overexpressed in NSCLC tumors. Manufacturing protocol yielded high transduction efficiency and T cell expansion within two weeks in 6/6 patients, consistent with PB-derived CAR T cells and on par with the optimal dosing requirements of an ICAM-1 CAR Phase I trial (NCT04420754). Notably, tdLN-CAR T cells demonstrated potent antitumor efficacy compared to the control in an aggressive NSCLC murine model (Median survival 103d vs 66d; respectively; p=0.006).

Conclusions: This represents the first reported use of T cells from tdLN for genetically engineered ACT. The data indicate that modifying antigen-experienced, stem-like T cells from tdLN with CAR is an efficient method to treat NSCLC and other cancers, especially solid tumors.

### REFERENCES

A. Im et al. (2016) Nature 537(7620):417-421.
B. Hudson et al. (2019) Immunity 51(6):1043-1058.e4.
C. Im et al. (2020) Proc Natl Acad Sci USA. 117(8):4292-4299.
D. Connolly et al. (2021) Science Immunology 6(64):eabg7836.
E. Villena-Vargas et al. (2022) International Association for the Study of Lung Cancer.
1. Onoi, K., Chihara, Y., Uchino, J., Shimamoto, T., Morimoto, Y., Iwasaku, M., . . . & Takayama, K. (2020). Immune checkpoint inhibitors for lung cancer treatment: a review. Journal of clinical medicine, 9(5), 1362.
2. Xu, Y., Li, H., & Fan, Y. (2021). Progression patterns, treatment, and prognosis beyond resistance of responders to immunotherapy in advanced non-small cell lung cancer. Frontiers in Oncology, 11, 642883.
3. Rizvi, N. A., Hellmann, M. D., Brahmer, J. R., Juergens, R. A., Borghaei, H., Gettinger, S., . . . & Antonia, S. (2016). Nivolumab in combination with platinum-based doublet chemotherapy for first-line treatment of advanced non-small-cell lung cancer. Journal of Clinical Oncology, 34(25), 2969.
4. Zimmermann, S., Peters, S., Owinokoko, T., & Gadgeel, S. M. (2018). Immune checkpoint inhibitors in the management of lung cancer. American Society of Clinical Oncology Educational Book, 38, 682-695.
5. Man, J., Millican, J., Mulvey, A., Gebski, V. & Hui, R. Response rate and survival at key timepoints with PD-1 blockade versus chemotherapy in PD-L1 subgroups: meta-analysis of metastatic NSCLC trials. JNCI Cancer Spectrum https://doi.org/10.1093/jncics/pkab012 (2021).
6. Rosenberg, S. A. et al. Durable complete responses in heavily pretreated patients with metastatic melanoma using T-cell transfer immunotherapy. Clin. Cancer Res. 17, 4550-4557 (2011).
7. Tran, E. et al. Cancer immunotherapy based on mutation-specific CD4+ T cells in a patient with epithelial cancer. Science 344, 641-645 (2014).
8. Stevanovid, S. et al. Complete regression of metastatic cervical cancer after treatment with human papillomavirus-targeted tumor-infiltrating T cells. J. Clin. Oncol. 33, 1543 (2015).
9. Tran, E. et al. T-cell transfer therapy targeting mutant KRAS in cancer. N. Engl. J. Med. 375, 2255-2262 (2016).
10. Duinkerken, C. W. et al. Sensorineural hearing loss after adoptive cell immunotherapy for melanoma using MART-1 specific T cells: a case report and its pathophysiology. Otol. Neurotol. 40, e674-e678 (2019).
11. Connolly, K. A., Kuchroo, M., Venkat, A., Khatun, A., Wang, J., William, I., . . . & Joshi, N. S. (2021). A reservoir of stem-like CD8+ T cells in the tumor-draining lymph node preserves the ongoing antitumor immune response. Science immunology, 6(64), eabg7836.
12. Huang, Q., Wu, X., Wang, Z., Chen, X., Wang, L., Lu, Y., Xiong, D., Liu, Q., Tian, Y., Lin, H., Guo, J., Wen, S., Dong, W., Yang, X., Yuan, Y., Yue, Z., Lei, S., Wu, Q., Ran, L., Xie, L., å¶ Ye, L. (2022). The primordial differentiation of tumor-specific memory CD8+ T cells as bona fide responders to PD-1/PD-L1 blockade in draining lymph nodes. Cell, 185(22), 4049-4066.e25.
13. Im, S. J., Hashimoto, M., Gerner, M. Y., Lee, J., Kissick, H. T., Burger, M. C., . . . & Ahmed, R. (2016). Defining CD8+ T cells that provide the proliferative burst after PD-1 therapy. Nature, 537(7620), 417-421.
14. Chu, F., Li, H. S., Liu, X., Cao, J., Ma, W., Ma, Y., . . . & Neelapu, S. S. (2019). CXCR5+CD8+ T cells are a distinct functional subset with an antitumor activity. Leukemia, 33(11), 2640-2653.
15. Im, S. J., Konieczny, B. T., Hudson, W. H., Masopust, D., & Ahmed, R. (2020). PD-1+ stemlike CD8 T cells are resident in lymphoid tissues during persistent LCMV infection. Proceedings of the National Academy of Sciences, 117(8), 4292-4299.
16. Villena-Vargas, J., Cruz, T. D., Markowitz, G., Singh, A., Martomo, S., Patel, J., . . . & Mittal, V. (2022). OA09. 05 Neoadjuvant IL-15-PDL1 Antibody Promotes T cell Memory and Decreases Metastatic Recurrence in Resectable NSCLC. Journal of Thoracic Oncology, 17(9)
17. Spolski, R., & Leonard, W. J. (2010). IL-21 and T follicular helper cells. International immunology, 22(1), 7-12. World Wide Web at doi.org/10.1093/intimm/dxpi112
18. Adusumilli, P. S., Cherkassky, L., Villena-Vargas, J., Colovos, C., Servais, E., Plotkin, J., . . . & Sadelain, M. (2014). Regional delivery of mesothelin-targeted CAR T cell therapy generates potent and long-lasting CD4-dependent tumor immunity. Science translational medicine, 6(261), 261ra151-261ra151.
19. Cornel AM, Mimpen I L, Nierkens S. MHC Class I Downregulation in Cancer: Underlying Mechanisms and Potential Targets for Cancer Immunotherapy. Cancers (Basel). 2020; 12(7):1760. Published 2020 Jul. 2. doi:10.3390/cancers12071760
20. Kotteas, E. A., Boulas, P., Gkiozos, I., Tsagkouli, S., Tsoukalas, G., & Syrigos, K. N. (2014). The intercellular cell adhesion molecule-1 (icam-1) in lung cancer: implications for disease progression and prognosis. Anticancer research, 34(9), 4665-4672.
21. Melis, M., Spatafora, M., Melodia, A., Pace, E., Gjomarkaj, M., Merendino, A. M., & Bonsignore, G. (1996). ICAM-1 expression by lung cancer cell lines: effects of upregulation by cytokines on the interaction with LAK cells. European Respiratory Journal, 9(9), 1831-1838.
22. Jung, M., Yang, Y., McCloskey, J. E., Zaman, M., Vedvyas, Y., Zhang, X., Stefanova, D., Gray, K. D., Min, I. M., Zarnegar, R., Choi, Y. Y., Cheong, J. H., Noh, S. H., Rha, S. Y., Chung, H. C., & Jin, M. M. (2020). Chimeric Antigen Receptor T Cell Therapy Targeting ICAM-1 in Gastric Cancer. Molecular therapy oncolytics, 18, 587-601. World Wide Web at doi.org/10.1016/j.omto.2020.08.009
23. Yang, Y., McCloskey, J. E., Yang, H., Puc, J., Alcaina, Y., Vedvyas, Y., Gomez Gallegos, A. A., Ortiz-Sánchez, E., de Stanchina, E., Min, I. M., von Hofe, E., & Jin, M. M. (2021). Bispecific CAR T Cells against EpCAM and Inducible ICAM-1 Overcome Antigen Heterogeneity and Generate Superior Antitumor Responses. Cancer immunology research, 9(10), 1158-1174. World Wide Web at doi.org/10.1158/2326-6066.CIR-21-0062
24. Park, S., Shevlin, E., Vedvyas, Y. et al. Micromolar affinity CAR T cells to ICAM-1 achieves rapid tumor elimination while avoiding systemic toxicity. Sci Rep 7, 14366 (2017). World Wide Web at doi.org/10.1038/s41598-017-14749-3
25. Pauken, K. E., Shahid, O., Lagattuta, K. A., Mahuron, K. M., Luber, J. M., Lowe, M. M., . . . & Singer, M. (2021). Single-cell analyses identify circulating anti-tumor CD8 T cells and markers for their enrichment. Journal of Experimental Medicine, 218
26. Herndler-Brandstetter, D., Ishigame, H., Shinnakasu, R., Plajer, V., Stecher, C., Zhao, J., . . . & Flavell, R. A. (2018). KLRG1+ effector CD8+ T cells lose KLRG1, differentiate into all memory T cell lineages, and convey enhanced protective immunity. Immunity, 48(4), 716-729
27. Chow, A., Uddin, F. Z., Liu, M., Dobrin, A., Nabet, B. Y., Mangarin, L., Lavin, Y., Rizvi, H., Tischfield, S. E., Quintanal-Villalonga, A., Chan, J. M., Shah, N., Allaj, V., Manoj, P., Mattar, M., Meneses, M., Landau, R., Ward, M., Kulick, A., Kwong, C., . . . Rudin, C. M. (2023). The ectonucleotidase CD39 identifies tumor-reactive CD8⁺ T cells predictive of immune checkpoint blockade efficacy in human lung cancer. Immunity, 56(1), 93-106.e6.

### INCORPORATION BY REFERENCE

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

A number of embodiments of the invention have been described. Nevertheless, it will be understood that various modifications may be made without departing from the spirit and scope of the invention. Accordingly, other embodiments are within the scope of the following claims. Unless defined otherwise, all technical and scientific terms used herein have the same meanings as commonly understood by one of skill in the art to which the disclosed invention belongs.

Those skilled in the art will recognize, or be able to ascertain using no more than routine experimentation, many equivalents to the specific embodiments of the invention described herein. Such equivalents are intended to be encompassed by the following claims.

What is claimed is:

1. A lymphocyte from a tumor-draining lymph node (TDLN) of a subject afflicted with a cancer, wherein the lymphocyte comprises a CAR polypeptide comprising:
a) at least one intracytoplasmic signaling region comprising a cluster of differentiation 3 zeta (CD3ζ) domain, and
b) an antigen binding domain specific for a cancer antigen.

2. The lymphocyte of claim 1, wherein the CAR polypeptide further comprises at least one costimulatory region,
optionally wherein the at least one costimulatory region comprises
a) a cluster of differentiation 28 (CD28) domain,
b) a 4-1BB domain, or
c) both a) and b).

3. The lymphocyte of claim 1 or 2, wherein the CAR polypeptide further comprises a cluster of differentiation 8 (CD8) hinge transmembrane domain.

4. The lymphocyte of any one of claims 1-3, wherein the cancer antigen is selected from ICAM-1, GD2, Mesothelin, HER2, EGFR, PSMA, MUC1, L1-CAM, CD276 (B7-H3), CD44v6, IL13Rα2, EpCAM, FAP, CD133, ROR1, CD24, B7-H4, NKG2D ligands, CD47, GPC3, Claudin 18.2, and EGFRvIII.

5. The lymphocyte of claim 4, wherein the cancer antigen is ICAM-1.

6. The lymphocyte of any one of claims 1-5, wherein the antigen-binding domain is a single chain fragment variable (svFv) that binds ICAM-1 or an I domain of the αL subunit of lymphocyte function-associated antigen-1 (LFA1).

7. The lymphocyte of any one of claims 1-6, wherein the TDLN is resected from a benign tumor.

8. The lymphocyte of any one of claims 1-7, wherein the TDLN is resected from a subject at an early-stage cancer or an advanced-stage cancer.

9. The lymphocyte of any one of claims 1-8, wherein the TDLN is resected from a subject treated prior to or concurrently with at least one checkpoint inhibitor.

10. The lymphocyte of claim 9, wherein the at least one checkpoint inhibitor is a PD-1 inhibitor (e.g., an anti-PD-1 antibody), a PD-L1 inhibitor (e.g., an anti-PD-L1 antibody), KD033, or any combination of two or more thereof.

11. The lymphocyte of claim 10, wherein the PD-1 inhibitor is selected from MP-514 (MEDI0680), nivolimumab, cemiplimab, pembrolizumab, dostarlimab, Vopratelimab (JTX-4014), Spartalizumab (PDR001), Camrelizumab (SHR1210), Sintilimab (IBI308), Tislelizumab (BGB-A317), Toripalimab (JS 001), INCMGA00012 (MGA012), and AMP-224.

12. The lymphocyte of claim 10, wherein the PD-L1 inhibitor is selected from atezolizumab, durvalumab, avelumab, KN035, Cosibelimab (CK-301), AUNP12, CA-170, and BMS-986189.

13. The lymphocyte of any one of claims 1-12, wherein the lymphocyte is a T lymphocyte, a cytotoxic T lymphocyte (CTL), a regulatory T cell, an αβT cell, γδT cell, or any combination thereof.

14. The lymphocyte of of any one of claims 1-13, wherein the lymphocyte is a T lymphocyte.

15. The lymphocyte of any one of claims 1-14, wherein the lymphocyte expresses CD8 or CD4.

16. The lymphocyte of any one of claims 1-15, wherein the lymphocyte expresses PD-1 and/or CXCR5.

17. The lymphocyte of any one of claims 1-16, wherein the lymphocyte expresses CD8, PD-1, and CXCR5.

18. The lymphocyte of any one of claims 1-17, wherein the lymphocyte expresses at least one cell surface protein selected from CD62L, CD45RA, CCR7, CD28, IL-7Rα, CXCR3, and CD95.

19. The lymphocyte of of any one of claims 1-18, wherein the lymphocyte does not express CD45RO.

20. The lymphocyte of any one of claims 1-19, wherein the cancer is a solid tumor.

21. The lymphocyte of any one of claims 1-20, wherein the cancer is selected from non-small cell lung cancer (NSCLC), gastric cancer, Melanoma, Breast cancer, Lung cancer, Esophageal cancer, Gastric cancer, Pancreatic cancer, Liver cancer, Colorectal cancer, Anal cancer, Cervical cancer, Ovarian cancer, Endometrial cancer, Prostate cancer, Bladder cancer, Kidney cancer, Testicular cancer, Penile cancer, Head and neck cancer (including oral, pharyngeal, and laryngeal cancers), Thyroid cancer, metastatic thyroid carcinoma, Soft tissue sarcomas, and Neuroendocrine tumors, optionally wherein the cancer is selected from NSCLC, thyroid cancer, and metastatic thyroid carcinoma.

22. A pharmaceutical composition comprising the lymphocyte of any one of claims 1-21.

23. The pharmaceutical composition of claim 22, wherein the pharmaceutical composition comprises at least about 1×10\hat{ }7 cells, optionally at least about 1×10\hat{ }8 cells

24. A method of treating a subject afflicted with a cancer, the method comprising administering to the subject (a) a lymphocyte from a tumor-draining lymph node, (b) the lymphocyte of any one of claims 1-21, (c) the pharmaceutical composition of claim 22 or 23, or (d) any combination of two or more selected from (a)-(c).

25. The method of claim 24, wherein the lymphocyte autologous or allogeneic to the subject.

26. The method of claim 24 or 25, wherein the subject is administered with a single dose of the lymphocyte or pharmaceutical composition.

27. The method of claim 24 or 25, wherein the subject is administered with at least two doses of the lymphocyte or pharmaceutical composition.

28. The method of any one of claims 24-27, wherein the subject is treated conjointly with at least one additional cancer therapy.

29. The method of claim 28, wherein the subject is administered with the at least one additional cancer therapy concomitant with, prior to, or following the administration of the lymphocyte or the pharmaceutical composition comprising the lymphocyte.

30. The method of claim 28 or 29, wherein the at least one additional cancer therapy is selected from immunotherapy, checkpoint inhibitors, cancer vaccines, chemotherapy, radiation therapy, and surgery, optionally a checkpoint inhibitors.

31. The method of any one of claims 24-30, wherein the at least one additional cancer therapy is a PD-1 inhibitor (e.g., an anti-PD-1 antibody), a PD-L1 inhibitor (e.g., an anti-PD-L1 antibody), KD033, or any combination of two or more thereof.

32. The method of claim 31, wherein the PD-1 inhibitor is selected from MP-514 (MEDI0680), nivolimumab, cemiplimab, pembrolizumab, dostarlimab, Vopratelimab (JTX-4014), Spartalizumab (PDR001), Camrelizumab (SHR1210), Sintilimab (IBI308), Tislelizumab (BGB-A317), Toripalimab (JS 001), INCMGA00012 (MGA012), and AMP-224.

33. The method of claim 31, wherein the PD-L1 inhibitor is selected from atezolizumab, durvalumab, avelumab, KN035, Cosibelimab (CK-301), AUNP12, CA-170, and BMS-986189.

34. The method of any one of claims 24-33, wherein the cancer is selected from non-small cell lung cancer (NSCLC), gastric cancer, Melanoma, Breast cancer, Lung cancer, Esophageal cancer, Gastric cancer, Pancreatic cancer, Liver cancer, Colorectal cancer, Anal cancer, Cervical cancer, Ovarian cancer, Endometrial cancer, Prostate cancer, Bladder cancer, Kidney cancer, Testicular cancer, Penile cancer, Head and neck cancer (including oral, pharyngeal, and laryngeal cancers), Thyroid cancer, metastatic thyroid carcinoma, Soft tissue sarcomas, and Neuroendocrine tumors, optionally wherein the cancer is selected from NSCLC, thyroid cancer, and metastatic thyroid carcinoma.

35. The method of any one of claims 1-34, wherein the subject is a mammal, optionally a mouse, a dog, a cat, or a human.
